(19) United States
(12) Patent Application Publication
(10) Pub. No.: US 2025/0345463 A1
(43) Pub. Date: Nov. 13, 2025

(54) GENE THERAPY FOR MONOGENIC DIABETES

(71) Applicant: UNIVERSITAT AUTÒNOMA DE BARCELONA, Cerdanyola del Vallès (ES)

(72) Inventors: Maria Fatima BOSCH TUBERT, Cerdanyola del Valles (ES); Veronica JIMENEZ CENZANO, Sabadell (ES); Miquel GARCIA MARTINEZ, Terrassa, Barcelona (ES); Estefania CASANA LORENTE, Sabadell, Barcelona (ES)

(73) Assignee: UNIVERSITAT AUTÒNOMA DE BARCELONA, Cerdanyola del Vallès (ES)

(21) Appl. No.: 18/260,344

(22) PCT Filed: Jan. 27, 2022
(86) PCT No.: PCT/EP2022/051899

§ 371 Date: Jul. 5, 2023

(30) Foreign Application Priority Data
Jan. 30, 2021 (EP) 21382079.8

(51) Int. Cl. A61K 48/00; C07K 14/47; C12N 15/86

(52) U.S. Cl. CPC A61K 48/0058; C07K 14/4702; C12N 15/86; C12N 2750/14143

(57) ABSTRACT

Described herein is a gene construct comprising a nucleotide sequence encoding a hepatocyte nuclear factor (HNF) such as HNF1A. Aspects described herein may be used in the treatment of maturity-onset diabetes of the young (MODY).

### FIELD

Aspects and embodiments described herein relate to the field of medicine, particularly gene therapy for monogenic diabetes.

### BACKGROUND

Maturity-onset diabetes of the young (MODY) comprises a heterogeneous group of monogenic disorders characterized by beta-cell dysfunction (impaired insulin secretion) with minimal or no defects in insulin action. MODYs are a rare cause of diabetes (1-2% of all cases of diabetes) (Fajans, S. S. et al. (2011). Diabetes Care, 34, 1878-84), with onset of hyperglycemia at an early age (generally before 25 years) (American Diabetes Association (2014). Diabetes Care, 37 Suppl 1, S81-90). MODY3 is the most common type of MODY and is caused by mutations in the gene encoding for the transcription factor hepatocyte nuclear factor (HNF)1A (Anik. A (2015). J. Pediatr. Endocrinol. Metab. 28, 251-63). MODY3 patients are typically normoglycemic in childhood, but mutations in the HNF1A genes cause progressive pancreatic beta-cell dysfunction that results in hyperglycemia, which is usually diagnosed between the second and fifth decades of life (Thanabalasingham, G. et al. (2011). BMJ, 343, d6044). Consequently, MODY3 patients are at risk of development of the full spectrum of microvascular and macrovascular complications associated with diabetes (Anik. A (2015). J. Pediatr. Endocrinol. Metab. 28, 251-63, Thanabalasingham, G. et al. (2011). BMJ, 343, d6044).

If diagnosed, MODY3 patients are treated for decades with sulfonylureas (Fajans, S. S. et al. (1993). Diabetes Care, 16, 1254-61; Pearson, E. R. et al. (2003). Lancet (London, England), 362, 1275-81). Sulfonylureas act by bypassing the functional defect present in the beta-cells of MODY3 patients, acting downstream of the metabolic steps that lead to insulin secretion (Pearson, E. R. et al. (2003). Lancet, 362, 1275-81). However, these patients develop unresponsiveness to sulfonylureas after 3-25 years of treatment due to gradual deterioration of their insulin secretion capacity, as a result of progressive glucose-induced beta-cell damage (Fajans, S. S. et al. (1993). Diabetes Care, 16, 1254-61). Hence, a substantial proportion of MODY3 patients require insulin therapy later in life (Thanabalasingham, G. et al. (2011). BMJ, 343, d6044; Fajans, S. S. et al. (1993). Diabetes Care, 16, 1254-61). Moreover, sulfonylureas have a narrow therapeutic index, making hypoglycaemic risk a serious concern. Furthermore, recent clinical and observational studies have reported an increased risk of cardiovascular events and deaths associated with sulfonylurea treatment (Bannister, C. A. et al. (2014). Diabetes. Obes. Metab., 16, 1165-73; Pladevall, M. et al. (2016). BMC Cardiovasc. Disord., 16, 14; Phung, O. J. et al. (2013). Diabet. Med., 30, 1160-71), apparently because the vast majority of sulfonylureas bind to receptors located not only in beta-cells but also in extra-pancreatic tissues (such as myocardium and smooth muscle) (Singh, A. K. et al. (2016). Expert Rev. Clin. Pharmacol.). Therefore, there is a necessity to develop new therapies for MODY3.

An additional hurdle to the development of efficient therapies for MODY is the availability of animal models that reproduce the phenotype observed in patients. Currently, there are two different global HNF1A knockout mouse models. Although these animals display a diabetic phenotype, they also show multiple organ manifestations that are not observed in MODY3 patients. In contrast, the beta-cell-specific overexpression of dominant negative mutants of HNF1A in two different lines of transgenic mice closely recapitulates the beta-cell dysfunction and diabetes observed in MODY3, without extra-pancreatic phenotype. However, these lines cannot be used to assess the therapeutic potential of HNF1A overexpression or replacement therapies for MODY3 because in the engineered beta-cells the dominant negative mutants would sequester the wild-type form of the HNF1A protein. Moreover, in dominant negative models the mutant HNF1A protein may sequester other beta-cell proteins, affecting the observed phenotype. Thus, MODY3 mouse models that exhibit a similar patient's phenotype and permit the evaluation of all feasible future therapies are required.

In view of the above, there is still a need for new treatments for monogenic diabetes which do not have all the drawbacks of existing treatments. There is also still a need for suitable disease models to investigate such treatments, particularly replacement therapy or gene therapy treatments.

### SUMMARY

An aspect of the invention relates to a gene construct for expression in the pancreas comprising a nucleotide sequence encoding a hepatocyte nuclear factor (HNF), operably linked to: (a) a pancreas-specific promoter; or (b) a ubiquitous promoter and at least one target sequence of a microRNA expressed in non-pancreatic tissue. In some embodiments, a gene construct according to the invention is such that the pancreas-specific promoter is selected from the group consisting of the pancreas/duodenum homeobox protein 1 (Pdx1) promoter, neurogenin 3 (Ngn3) promoter, HNF promoters, elastase I promoter, amylase promoter, MafA promoter, insulin (Ins) promoter and derivatives thereof, preferably an insulin promoter or a derivative thereof. In some embodiments, the pancreas-specific promoter is a murine, canine or human insulin promoter or a derivative thereof, preferably a human or murine insulin promoter or a derivative thereof, more preferably a human insulin promoter or a derivative thereof. In some embodiments, the pancreas-specific promoter comprises, consists essentially of or consists of:
the nucleotides corresponding to positions −385 to −1 in the human insulin promoter (SEQ ID NO: 18); and/or
the nucleotide sequence of SEQ ID NO: 20, or a sequence having at least 60%, 70%, 80%, 90%, 95% or 99% sequence identity therewith.

In some embodiments, a gene construct according to the invention is such that the at least one target sequence of a microRNA is selected from those target sequences that bind to microRNAs expressed in heart and/or liver. In some embodiments, a gene construct according to the invention is such that the gene construct comprises at least one target sequence of a microRNA expressed in the liver and at least one target sequence of a microRNA expressed in the heart, preferably wherein a target sequence of a microRNA expressed in the heart is selected from SEQ ID NO's: 29-34 and a target sequence of a microRNA expressed in the liver is selected from SEQ ID NO's: 21-28, more preferably wherein the gene construct comprises a target sequence of microRNA-122a (SEQ ID NO: 21) and a target sequence of microRNA-1 (SEQ ID NO: 29). In some embodiments, a gene construct according to the invention is such that the HNF is an HNF1A. In some embodiments, a gene construct according to the invention is such that the nucleotide sequence encoding HNF1A is selected from the group consisting of:
(a) a nucleotide sequence encoding a polypeptide represented by an amino acid sequence comprising a sequence that has at least 60%, 70%, 80%, 90%, 95% or 99% sequence identity or similarity with the amino acid sequence of any one of SEQ ID NO: 1-11, 51;
(b) a nucleotide sequence that has at least 60%, 70%, 80%, 90%, 95% or 99% sequence identity with the nucleotide sequence of any one of SEQ ID NO: 12-15; and
(c) a nucleotide sequence the sequence of which differs from the sequence of a nucleotide sequence of (b) due to the degeneracy of the genetic code.

Another aspect of the invention relates to an expression vector comprising a gene construct of the invention. In some embodiments, an expression vector of the invention is such that the expression vector is a viral vector, preferably an adeno-associated viral vector. In some embodiments, an expression vector of the invention is such that the expression vector is an adeno-associated viral vector of serotype 1, 2, 3, 4, 5, 6, 7, 8, 9, rh10, rh8, Cb4, rh74, DJ, 2/5, 2/1, 1/2 or Anc80, preferably an adeno-associated viral vector of serotype 6, 8 or 9, more preferably an adeno-associated viral vector of serotype 8.

Another aspect of the invention relates to a pharmaceutical composition comprising a gene construct of the invention and/or an expression vector of the invention, optionally further comprising one or more pharmaceutically acceptable ingredients

Another aspect of the invention relates to a gene construct of the invention, an expression vector of the invention, or a pharmaceutical composition of the invention, for use as a medicament. In some embodiments, a gene construct for use of the invention, an expression vector for use of the invention, or a pharmaceutical composition for use of the invention is for use in the treatment of maturity onset diabetes of the young (MODY) or a condition associated therewith. In some embodiments, MODY is MODY3 or a condition associated therewith.

### DESCRIPTION

The present inventors have developed an animal disease model that closely recapitulates the human disease and that allows evaluation of treatment strategies including protein replacement and gene therapy treatments. Using this model, a gene therapy strategy based on hepatocyte nuclear factor 1A or HNF1A to counteract monogenic diabetes or maturity-onset diabetes of the young (MODY), in particular MODY3, was found. Particularly, as elaborated in the experimental part, the following unexpected advantages have been found. AAV-mediated HNF1A gene therapy mediates specific overexpression in the pancreas, particularly in the beta cells of the pancreas and exerts at least the following benefits:
Decreased hyperglycemia
Increased glucose tolerance
Maintenance of body weight

Given that the diabetic phenotype of MODY3 is due to mutations in genes that affect primarily beta-cell function, gene transfer of HNF1A to this cell type would be per se curative. Hence, significant benefit over existing therapeutic strategies or others under development may reasonably be expected. In vivo gene therapy based on adeno-associated viral vectors (AAV), offers the possibility of a one-time treatment, with the prospect of lifelong beneficial effects, as the production of the therapeutic protein for extended periods of time after a single administration of the gene therapy product has been repeatedly demonstrated in several animal models and humans (Mingozzi, F. et al. (2011). Nat. Rev. Genet., 12, 341-55; Grieger, J. C. et al. (2012). Methods Enzymol., 507, 229-54).

Accordingly, the aspects and embodiments of the present invention as described herein solve at least some of the problems and needs as discussed herein.

### Gene Construct

In a first aspect, there is provided a gene construct comprising a nucleotide sequence encoding a hepatocyte nuclear factor (HNF), operably linked to:
(a) a pancreas-specific promoter; or
(b) a ubiquitous promoter and at least one target sequence of a microRNA expressed in non-pancreatic tissue.

In some embodiments, a gene construct as described herein is for expression in a vertebrate, more preferably a mammal. In some embodiments, a gene construct as described herein is for expression in a pancreas, more preferably a mammalian pancreas.

As used herein, “for expression” or “suitable for expression” may mean that the gene construct includes one or more regulatory sequences, selected on the basis of the host cells such as pancreas cells of the vertebrate or mammal to be used for expression, which is operatively linked to the nucleotide sequence to be expressed. Preferably, host cells to be used for expression are human, murine or canine cells.

In any embodiment described herein, the term “promoter” may be replaced by “transcription regulatory sequence” or “regulatory sequence”. Definitions of the terms are provided in the “general information” section. A “gene construct” as described herein has its customary and ordinary meaning as understood by one of skill in the art in view of this disclosure. A “gene construct” can also be called “expression cassette” or “expression construct” and refers to a gene or a group of genes, including a gene that encodes a protein of interest, which is operably linked to a regulatory sequence that controls its expression. The part of this application entitled “general information” comprises more detail as to a “gene construct”. “Operably linked” as used herein is further described in the part of this application entitled “general information”.

In preferred embodiments, a gene construct as described herein is suitable for expression in a pancreas of a vertebrate, preferably in a mammalian pancreas, more preferably in a human, murine or canine pancreas. In more preferred embodiments, a gene construct as described herein is suitable for expression in a human pancreas. As used herein, “suitable for expression in a pancreas” may mean that the gene construct includes one or more regulatory sequences that directs expression of the nucleotide sequence to be expressed in said pancreas, preferably in a beta-cell of the islet of Langerhans or a complete islet of Langerhans. In some embodiments, a gene construct as described herein, particularly one that is for expression in the pancreas, refers to a gene construct which can direct expression of said nucleotide sequence in at least one cell of the pancreas and/or pancreatic islets. Preferably, said gene construct directs expression in at least 10%, 20%, 30%, 40%, 40%, 60%, 70%, 80%, 90%, 95%, 99% or 100% of cells of the pancreas and/or the pancreatic islets. A gene construct as described herein also encompasses gene constructs directing expression in a specific region or cellular subset of the pancreas and/or pancreatic islets. Accordingly, gene constructs as described herein may also direct expression in at least 10%, 20%, 30%, 40%, 40%, 60%, 70%, 80%, 90%, 95%, 99% or 100% of cells of the endocrine cells of the pancreatic islets. Expression may be assessed as described under the section entitled “general information”.

A gene construct according to the invention comprises a nucleotide sequence encoding a hepatocyte nuclear factor (HNF), which is a transcription factor, expressed in multiple tissues such as the liver and pancreas, associated with development and metabolic homeostasis of the organism. HNFs as described herein are preferably HNFs which contain a POU-homeodomain and/or HNFs that bind to DNA as homodimers. POU proteins are eukaryotic transcription factors containing a bipartite DNA binding domain referred to as the POU domain. The POU domain is a bipartite domain composed of two subunits separated by a non-conserved region of 15-55 aa. The N-terminal subunit is known as the POU-specific (POUs) domain (Interpro: IPR000327), while the C-terminal subunit is a homeobox domain (Interpro: IPR001356).

HNFs as described herein are preferably HNF1 family members, including HNF1A, HNF1B and their isoforms. In a preferred embodiment, an HNF as described herein is an HNF1A.

The skilled person understands that different HNF isoforms may exist and that the number of different HNF isoforms may vary depending on the organism and that any HNF isoform may be suitable for use in the invention. As a non-limiting example, the human HNF1A has 8 isoforms, namely isoforms a, b, c, 4, 5, 6, 7 (also known as inslVS8) and 8 (also known as delta 2), all of which are suitable. HNF1A, and particularly HNF1A isoform a, are advantageous. HNF1A isoform a is generally regarded as the canonical sequence.

A nucleotide sequence encoding an HNF as described herein may be derived from any HNF gene or HNF coding sequence, preferably an HNF gene or HNF coding sequence from human, murine or canine origin such as from human, mouse, rat or dog; or a mutated HNF gene or HNF coding sequence, preferably from human, murine or canine origin such as from human, mouse, rat or dog; or a codon optimized HNF gene or HNF coding sequence, preferably from human, murine or canine origin such as from human, mouse, rat or dog.

In some embodiments, a preferred nucleotide sequence encoding an HNF1A encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 1, 2, 3, 4, 5, 6, 7 or 8, more preferably with SEQ ID NO: 1. SEQ ID NO: 1 represents an amino acid sequence of human HNF1A isoform a. SEQ ID NO: 2 represents an amino acid sequence of human HNF1A isoform b. SEQ ID NO: 3 represents an amino acid sequence of human HNF1A isoform c. SEQ ID NO: 4 represents an amino acid sequence of human HNF1A isoform 4. SEQ ID NO: 5 represents an amino acid sequence of human HNF1A isoform 5. SEQ ID NO: 6 represents an amino acid sequence of human HNF1A isoform 6. SEQ ID NO: 7 represents an amino acid sequence of human HNF1A isoform 7 (also known as insIVS8). SEQ ID NO: 8 represents an amino acid sequence of human HNF1A isoform 8 (also known as delta 2).

In some embodiments, a preferred nucleotide sequence encoding an HNF1A encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 9, 10 or 51, more preferably with SEQ ID NO: 51. SEQ ID NO: 51 is the canonical mouse sequence. SEQ ID NO: 9 represents a computationally inferred amino acid sequence of murine HNF1A isoform H3BL72. SEQ ID NO: 10 represents an computationally inferred amino acid sequence of murine HNF1A isoform H3BKV2.

In some embodiments, a preferred nucleotide sequence encoding an HNF1A encodes a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with SEQ ID NO: 11. SEQ ID NO: 11 represents an amino acid sequence of canine HNF1A.

In some embodiments, a nucleotide sequence encoding an HNF1A present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with any sequence selected from the group consisting of SEQ ID NO's: 12 and 15. SEQ ID NO: 12 represents a nucleotide sequence encoding human HNF1A. SEQ ID NO: 15 represents a codon-optimized sequence of human HNF1A. Different isoforms may be formed by differential splicing.

In some embodiments, a nucleotide sequence encoding an HNF1A present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 13. SEQ ID NO: 13 represents a nucleotide sequence encoding murine HNF1A. Different isoforms may be formed by differential splicing.

In some embodiments, a nucleotide sequence encoding an HNF1A present in a gene construct according to the invention has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 14. SEQ ID NO: 14 represents a nucleotide sequence encoding canine HNF1A.

A description of “identity” or “sequence identity” and “similarity” or “sequence similarity” has been provided under the section entitled “general information”.

In some embodiments, there is provided a gene construct as described herein, wherein the nucleotide sequence encoding an HNF1A is selected from the group consisting of:
(a) a nucleotide sequence encoding a polypeptide represented by an amino acid sequence comprising a sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or similarity with the amino acid sequence of any one of SEQ ID NOs: 1-11 and 51, preferably SEQ ID NO: 1, 11 or 51, more preferably SEQ ID NO: 1 or 51, most preferably SEQ ID NO: 1;
(b) a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the nucleotide sequence of any one of SEQ ID NOs: 12-15, preferably SEQ ID NO: 12 or 13, more preferably SEQ ID NO: 12;
(c) a nucleotide sequence the sequence of which differs from the sequence of a nucleotide sequence of (b) due to the degeneracy of the genetic code.

In some embodiments, a nucleotide sequence encoding an HNF1A present in a gene construct according to the invention is a codon-optimized HNF1A sequence, preferably a codon-optimized human HNF1A sequence. In some embodiments, it has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 15. SEQ ID NO: 15 represents codon optimized nucleotide sequences encoding HNF1A amino acid sequence with SEQ ID NO: 1. A description of “codon optimization” has been provided under the section entitled “general information”.

An HNF, preferably an HNF1A, more preferably an HNF1A isoform a, encoded by the nucleotide sequences described herein exerts at least a detectable level of an activity as known to a person of skill in the art. As a non-limiting example, an activity of an HNF, preferably an HNF1A, preferably an HNF1A isoform a, will result in the transcription of downstream genes being modified, resulting in a detectable change in a phenotype such as, but not limited to, a reduction in hyperglycemia and improvement of glucose tolerance. These activities of an HNF could be assessed by methods known to a person of skill in the art, for example by using gene expression analysis to detect the expression of marker genes, or Electrophoretic Mobility Shift Assay (EMSA) to detect transcription factor binding to DNA. Suitable marker genes, which are target genes of HNF1A, may be selected from the group consisting of: Glut2 (Glucose transporter 2), L-pk (L-pyruvate kinase), NBAT (neuroblastoma associated transcript 1), Igf-1 (Insulin Like Growth Factor 1), Insi (insulin 1), Hnf4a (hepatocyte nuclear factor 4 alpha), Hnf1b (hepatocyte nuclear factor 1 beta), Pdx1 (pancreatic and duodenal homeobox 1) and Hnf3b (hepatocyte nuclear factor 3 beta), preferably Glut2 and L-pk. Alternatively, the change in a phenotype such as, but not limited to, a reduction in hyperglycemia and improvement of glucose tolerance may be monitored. Suitable methods are known to the skilled person and are for example described in the experimental section.

In some embodiments, the nucleotide sequence encoding an HNF, preferably an HNF1A, more preferably an HNF1A isoform a, is operably linked to a pancreas-specific promoter. A description of “pancreas-specific promoter” has been provided under the section entitled “general information”.

A promoter as used herein encompasses derivatives of promoters and should exert at least an activity of a promoter as known to a person of skill in the art (especially when the promoter sequence is described as having a minimal identity percentage with a given SEQ ID NO). Preferably, a promoter described as having a minimal identity percentage with a given SEQ ID NO should control transcription of the nucleotide sequence to which it is operably linked (i.e. at least a nucleotide sequence encoding a HNF) as assessed in an assay known to a person of skill in the art. For example, such assay could involve measuring expression of the transgene. Expression may be assessed as described under the section entitled “general information”.

In a preferred embodiment, the pancreas-specific promoter is a pancreatic islet-specific promoter, more preferably a beta-cell-specific promoter. Preferably, said promoters are derived from human, murine or canine genes such as from human, mouse, rat or dog genes. In some embodiments, a pancreas-, pancreatic islet- and/or beta cell-specific promoter as described herein is selected from the group consisting of the pancreas/duodenum homeobox protein 1 (Pdx1) promoter, neurogenin 3 (Ngn3) promoter, HNF promoters, elastase I promoter, amylase promoter, MafA promoter, insulin (Ins) promoter and derivatives thereof, preferably the pancreas-, pancreatic islet- and/or beta cell-specific promoter is an insulin promoter or a derivative thereof.

Derivatives of promoters as described herein comprise promoters that have been mutated as to differentiate the directed expression of the transgenes operably linked to said promoters as compared to the non-mutated promoters, which can be increased or decreased, preferably decreased. Methods of mutating nucleotide sequences are known to the skilled person and can comprise any of introduction of single nucleotide polymorphisms, nucleotide insertions and nucleotide deletions. Insulin promoters and their derivatives are particularly useful for expression of gene constructs in mammalian beta-cells. The skilled person understands that derivatives of promoters can also encompass promoters that have been shortened (by nucleotide deletions) or elongated (by nucleotide insertions) compared to their wild-type sequences, with shortened promoters being preferred.

In some embodiments, a derivative of an insulin promoter may be a fragment of an insulin promoter.

In some embodiments, a fragment of an insulin promoter comprises, consists essentially of or consists of:
the nucleotides corresponding to positions −385 to +24 in the human insulin promoter (SEQ ID NO: 18) (for example as described by Fukazawa et al. Experimental Cell Research 2006; 312:3404-3412), or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity sequence identity therewith; or
the nucleotide sequence of SEQ ID NO: 19, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In preferred embodiments, a fragment of an insulin promoter comprises, consists essentially of or consists of:
the nucleotides corresponding to positions −385 to −1 in the human insulin promoter (SEQ ID NO: 18) (for example as described by Fukazawa et al. Experimental Cell Research 2006; 312:3404-3412) (also denoted as “hlns385” herein), or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith; or
the nucleotide sequence of SEQ ID NO: 20, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

The inventors have surprisingly found that this fragment wherein the nucleotides+1 to +24 are deleted is associated with unexpected advantages when said promoters are used to direct expression of HNF transgenes such as HNF1A, as described in Example 3. The skilled person understands that the equivalent nucleotides in homologous insulin promoters can be derived by alignment of the hlNS promoter fragment of SEQ ID NO: 19 or 20 with the promoter in question, using global alignment tools known in the art and further elaborated upon in the “general information” section.

In some embodiments, a derivative of a promoter as described herein, such as a fragment of an insulin promoter as described herein, has reduced promoter activity compared to the wildtype and full-length promoter, such as the the full-length insulin promoter. In some embodiments, reduced promoter activity may mean about 10%, 20%, 30%, 40%, 50%, 60%, 70%, 80%, 90%, 95%, 98% or 99% reduction, preferably about 95%. In other words, the level of expression generated from a derivative such as a fragment of a full-lenght human insulin promoter as described herein, may be reduced by about 10%, 20%, 30%, 40%, 50%, 60%, 70%, 80%, 90%, 95%, 98%, or 99%, preferably by about 95%, compared to the level of expression generated from the wildtype and full-length promoter. Level of expression may be expressed on the basis of mRNA or protein levels. In some embodiments, reduced promoter activity or a reduced level of expression may mean about 10%, 20%, 30%, 40%, 50%, 60%, 70%, 80%, 90%, 95%, 98% or 99% reduction in mRNA level relative to the mRNA obtained with the full-length human insulin promoter (hlns1.9), preferably about 95%. In some embodiments, reduced promoter activity or a reduced level of expression may mean about 10%, 20%, 30%, 40%, 50%, 60%, 70%, 80%, 90%, 95%, 98% or 99% reduction in protein level relative to the protein obtained with the full-length human insulin promoter (hlns1.9), preferably about 95%. In some embodiments, promoter activity or level of expression may be measured by a marker gene, such as gfp. In some embodiments, reduced promoter activity or a reduced level of expression may mean about 75-99%, preferably about 85-99%, more preferably about 90-99%, even more preferably about 92-98%, most preferably about 94-96% reduction in promoter activity or level of expression compared to the full-length human insulin promoter (hlns1.9). Promoter activity and expression can be measured by methods known in the art, as described elsewhere herein and in the examples.

In some embodiments, an insulin promoter or a derivative thereof is selected from the group consisting of a human, murine (including rat or mouse) or canine (including dog) insulin promoter or a derivative thereof, preferably a human or murine (including rat or mouse) insulin promoter or a derivative thereof, more preferably a human insulin promoter or a derivative thereof. In some embodiments, an insulin promoter or a derivative thereof is selected from a rat insulin promoter or a derivative thereof and a human insulin promoter or a deriviative thereof.

In some embodiments, a rat insulin promoter as described herein may be rat insulin promoter 1 (RIPI) or a rat insulin promoter 2 (RIPII). A rat insulin promoter 1 may comprise, consist essentially of or consist of the nucleotide sequence of SEQ ID NO: 16, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith. A rat insulin promoter 2 may comprise, consist essentially of or consist of the nucleotide sequence of SEQ ID NO: 17, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In some embodiments, a human insulin promoter as described herein may be a full-lenght human insulin promoter (also denoted herein as hlNS1.9) or a derivative thereof. An hlns 1.9 promoter may comprise, consist essentially of or consist of the nucleotide sequence of SEQ ID NO: 18, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In a preferred embodiment, a human insulin promoter as described herein may be a derivative, preferably a fragment, of a full-length human insulin promoter. In some embodiments, a human insulin promoter comprises, consists essentially of or consists of the sequence of SEQ ID NO: 19, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith. In some embodiments, a human insulin promoter comprises, consists essentially of or consists of the sequence of SEQ ID NO: 20, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

SEQ ID NO: 19 represents positions −385 to +24 in the human insulin promoter (for example as described by Fukazawa et al. Experimental Cell Research 2006; 312:3404-3412), and SEQ ID NO: 20 represents positions −385 to −1 in the human insulin promoter as described by Fukazawa et al. Experimental Cell Research 2006; 312:3404-3412.

Other suitable fragments of human insulin promoters are described by Kuroda, Akio et al. “Insulin gene expression is regulated by DNA methylation.” PloS one vol. 4,9 e6953. 9 Sep. 2009.

In some embodiments, a derivative such as a fragment of a full-length human insulin promoter as described herein, has reduced promoter activity compared to the full-length human insulin promoter (hlns1.9). In some embodiments, reduced promoter activity may mean about 10%, 20%, 30%, 40%, 50%, 60%, 70%, 80%, 90%, 95%, 98% or 99% reduction, preferably about 95%. In other words, the level of expression generated from a derivative such as a fragment of a full-lenght human insulin promoter as described herein, may be reduced by about 10%, 20%, 30%, 40%, 50%, 60%, 70%, 80%, 90%, 95%, 98%, or 99%, preferably by about 95%, compared to the level of expression generated from the full-lenght human insulin promoter (hlns1.9). Level of expression may be expressed on the basis of mRNA or protein levels. In some embodiments, reduced promoter activity or a reduced level of expression may mean about 10%, 20%, 30%, 40%, 50%, 60%, 70%, 80%, 90%, 95%, 98% or 99% reduction in mRNA level relative to the mRNA obtained with the full-lenght human insulin promoter (hlns1.9), preferably about 95%. In some embodiments, reduced promoter activity or a reduced level of expression may mean about 10%, 20%, 30%, 40%, 50%, 60%, 70%, 80%, 90%, 95%, 98% or 99% reduction in protein level relative to the protein obtained with the full-lenght human insulin promoter (hlns1.9), preferably about 95%. In some embodiments, promoter activity or level of expression may be measured by a marker gene, such as gfp. In some embodiments, reduced promoter activity or a reduced level of expression may mean about 75-99%, preferably about 85-99%, more preferably about 90-99%, even more preferably about 92-98%, most preferably about 94-96% reduction in promoter activity or level of expression compared to the full-lenght human insulin promoter (hlns1.9). Promoter activity and expression can be measured by methods known in the art, as described elsewhere herein and in the examples.

In some embodiments, a derivative such as a fragment of any promoter as described herein, preferably an insulin promoter as described herein, may have a length between 100-1000 bp orbetween 200-800 bp, preferably between 300-500 bp, more preferably between 350-420 bp and even more preferably between 370-400 bp. In some embodiments, a derivative such as a fragment of any promoter as described herein, preferably an insulin promoter as described herein, may have a length of at most 1000 bp or at most 800 bp, preferably at most 500 bp, more preferably at most 420 bp, even more preferably at most 400 bp.

HNFs described herein can be operably linked to multiple copies of promoters described herein. HNFs can be operably linked to 1, 2, 3, 4 or 5 copies of promoter sequences. The skilled person understands that the copies do not necessarily need to derive from the same promoter and that combinations of different promoter sequences may be used. The promoter copies may correspond to full-length promoters or promoter fragments as well as their derivatives. In some embodiments, an HNF, preferably an HNF1A, more preferably an HNF1A isoform a, is operably linked to at most 2 copies, or preferably a single copy of any promoter described herein, such as a fragment of an insulin promoter comprising, consisting essentially of or consisting of:
the nucleotides corresponding to positions −385 to −1 in the human insulin promoter (SEQ ID NO: 18) (for example as described by Fukazawa et al. Experimental Cell Research 2006; 312:3404-3412) (SEQ ID NO: 20, also denoted as “hlns385” herein); or
the nucleotide sequence of SEQ ID NO: 20, or a sequence having at least 60%, 70%, 80%, 90%, 95% or 99% sequence identity therewith.

In some embodiments, a pancreas-specific promoter as described herein refers to a pancreas-, pancreatic islet- and/or beta-cell-specific promoter which can direct expression of said nucleotide sequence in at least one cell of the pancreas and/or pancreatic islets. Preferably, said promoter directs expression in at least 10%, 20%, 30%, 40%, 40%, 60%, 70%, 80%, 90%, or 100% of cells of the pancreas and/or the pancreatic islets. A pancreas- and/or pancreatic islet- and/or beta-cell-specific promoter, as used herein, also encompasses promoters directing expression in a specific region or cellular subset of the pancreas and/or pancreatic islets. Accordingly, pancreas- and/or pancreatic islet- and/or beta-cell-specific promoters as described herein may also direct expression in at least 10%, 20%, 30%, 40%, 40%, 60%, 70%, 80%, 90%, or 100% of cells of the endocrine cells of the pancreatic islets. Expression may be assessed as described under the section entitled “general information”.

In some embodiments, the nucleotide sequence encoding an HNF, preferably an HNF1A, more preferably an HNF1A isoform a, is operably linked to a ubiquitous promoter.

In some embodiments, the nucleotide sequence encoding an HNF, preferably an HNF1A, more preferably an HNF1A isoform a, is operably linked to at least one target sequence of a microRNA expressed in a non-pancreatic tissue.

In some embodiments, the nucleotide sequence encoding an HNF, preferably an HNF1A, more preferably an HNF1A isoform a, is operably linked to a ubiquitous promoter and at least one target sequence of a microRNA expressed in a non-pancreatic tissue.

The term “non-pancreatic tissue” as used herein refers to organs and/ortissues otherthan the pancreas, as customarily and ordinarily understood by the skilled person. Non-limiting examples of non-pancreatic tissues are the liver, CNS, brain, adipose tissue, skeletal muscle, heart, kidney, colon, hematopoietic tissue, lung, ovary, spleen, stomach, testis and others, preferably the liver and the heart.

A description of “ubiquitous promoter”, “operably linked” and “microRNA” has been provided under the section entitled “general information”. A “target sequence of a microRNA expressed in a non-pancreatic tissue” or “target sequence binding to a microRNA expressed in a non-pancreatic tissue” or “binding site of a microRNA expressed in a non-pancreatic tissue” as used herein refers to a nucleotide sequence which is complementary or partially complementary to at least a portion of a microRNA expressed in said non-pancreatic tissue, as described elsewhere herein.

When a nucleotide sequence encoding an HNF as described herein is operably linked to at least one target sequence of a microRNA expressed in a non-pancreatic tissue, this may be to prevent unwanted expression in said non-pancreatic tissue.

For several tens to hundreds of organisms, including both plants and non-human animals, comprehensive miRNA knowledge has been established, including miRNA sequences and information on distribution of expression of each miRNA among different cells, tissues and organs. For example, miRBase comprises miRNA sequences from more than 270 organisms across invertebrates, vertebrates and plants. miRBase is the primary public repository and online resource for microRNA sequences and annotation. The miRBase website provides a wide-range of information on published microRNAs, including their sequences, their biogenesis precursors, genome coordinates and context, literature references, deep sequencing expression data and community-driven annotation. miRBase is available at http://www.mirbase.org, described in Kozomara et al. miRBase: from microRNA sequences to function, Nucleic Acids Research, Volume 47, Issue D1, 8 Jan. 2019, Pages D155-D162, incorporated herein by reference).

For animals, the following databases and publications including sequences and expression information are available:
miRBase, available at http://www.mirbase.org, described in Kozomara et al. miRBase: from microRNA sequences to function, Nucleic Acids Research, Volume 47, Issue D1, 8 Jan. 2019, Pages D155-D162, incorporated herein by reference.
miRNEST, available at http://rhesus.amu.edu.pl/mirnest/copy/, described in Szczesniak MW, Makalowska I (2014) miRNEST 2.0: a database of plant and animal microRNAs. Nucleic Acids Res. 42:D74-D77, incorporated herein by reference.
Isakova et al. A mouse tissue atlas of small noncododing RNA. PNAS 2020; 117(41:25634-25645, incorporated herein by reference.
Ludwig, Nicole, et al. Distribution of miRNA expression across human tissues. Nucleic acids research 44.8 (2016): 3865-3877 (also available at https://genome.ucsc.edu/and https://ccb-web.cs.uni-saarland.de/tissueatlas/), incorporated herein by reference.
RATEmiRs, available at https://connect.niehs.nih.gov/ratemirs/, described in Bushel, P. R., Caiment, F., Wu, H. et al. RATEmiRs: the rat atlas of tissue-specific and enriched miRNAs database. BMC Genomics 19, 825 (2018), incorporated herein by reference.
de Rie, D., Abugessaisa, I., Alam, T. et al. An integrated expression atlas of miRNAs and their promoters in human and mouse. Nat Biotechnol 35, 872-878 (2017), incorporated herein by reference.
Londin, E., Loher, P., Telonis, A. G., Quann, K., Clark, P., Jing, Y., Hatzimichael, E., Kirino, Y., Honda, S., Lally, M., et al. (2015a). Analysis of 13 cell types reveals evidence for the expression of numerous novel primate- and tissue-specific microRNAs. Proc. Natl. Acad. Sci. 112, E1106-E1115, incorporated herein by reference.
McCall, M. N., Kim, M.-S., Adil, M., Patil, A. H., Lu, Y., Mitchell, C. J., Leal-Rojas, P., Xu, J., Kumar, M., Dawson, V. L., et al. (2017). Toward the human cellular microRNAome. Genome Res. 27, 1769-1781, incorporated herein by reference.

All of the microRNAs and microRNA target sequences as well as the information about their expression in different cells, tissues and organs as disclosed in the above publications and databases is expressly incorporated herein by reference.

In addition to the above, a skilled person could identify further miRNAs including cell-, tissue- and organ-specific miRNAs in any desired plant or non-human animal organism based on available methods and techniques, such as RNASeq. See for example the methods, in particular RNASeq-based methods, described in any of the publications cited above.

In some embodiments, one, two, three, four, five, six, seven or eight copies of the target sequence of a microRNA are present in the gene construct of the invention. A preferred number of copies of a target sequence of a microRNA is four.

In some embodiments, the at least one target sequence of a microRNA is selected from those target sequences that bind to microRNAs expressed in heart and/or liver, preferably of a mammal.

In some embodiments, the nucleotide sequence encoding an HNF, preferably an HNF1A, more preferably an HNF1A isoform a, is operably linked to at least one target sequence of a microRNA expressed in the liver and at least one target sequence of a microRNA expressed in the heart. In some embodiments, the nucleotide sequence encoding an HNF, preferably an HNF1A, more preferably an HNF1A isoform a, is operably linked to a ubiquitous promoter and at least one target sequence of a microRNA expressed in the liver and at least one target sequence of a microRNA expressed in the heart. A target sequence of a microRNA expressed in the liver is preferably selected from SEQ ID NO's: 21-28, more preferably SEQ ID NO: 21 (microRNA-122a) and a target sequence of a microRNA expressed in the heart is preferably selected from SEQ ID NO's: 29-34, more preferably SEQ ID NO: 29 (microRNA-1).

A “target sequence of a microRNA” or “target sequence binding to a microRNA” or “binding site of a microRNA”, or similar expressions, as used herein, refer to a nucleotide sequence which is complementary or partially complementary to at least a portion of a microRNA. A “target sequence of a microRNA expressed in the liver” or “target sequence binding to a microRNA expressed in the liver” or “binding site of a microRNA expressed in the liver”, or similar expressions, as used herein, refer to a nucleotide sequence which is complementary or partially complementary to at least a portion of a microRNA expressed in the liver. Similarly, a “target sequence of a microRNA expressed in the heart” or “target sequence binding to a microRNA expressed in the heart” or “binding site of a microRNA expressed in the heart”, or similar expressions, as used hereins refers to a nucleotide sequence which is complementary or partially complementary to at least a portion of a microRNA expressed in the heart. A portion of a microRNA, for example a portion of a microRNA expressed in the liver or a portion of a microRNA expressed in the heart, as described herein, means a nucleotide sequence of at least four, at least five, at least six or at least seven consecutive nucleotides of said microRNA. The binding site sequence can have perfect complementarity to at least a portion of an expressed microRNA, meaning that the sequences are a perfect match without any mismatch occurring. Alternatively, the binding site sequence can be partially complementary to at least a portion of an expressed microRNA, meaning that one mismatch in four, five, six or seven consecutive nucleotides may occur. Partially complementary binding sites preferably contain perfect or near perfect complementarity to the seed region of the microRNA, meaning that no mismatch (perfect complementarity) or one mismatch per four, five, six or seven consecutive nucleotides (near perfect complementarity) may occur between the seed region of the microRNA and its binding site. The seed region of the microRNA consists of the 5′ region of the microRNA from about nucleotide 2 to about nucleotide 8 of the microRNA. The portion as described herein is preferably the seed region of said microRNA. Degradation of the messenger RNA (mRNA) containing the target sequence for a microRNA such as a microRNA expressed in the liver or a microRNA expressed in the heart may be through the RNA interference pathway or via direct translational control (inhibition) of the mRNA. This invention is in no way limited by the pathway ultimately utilized by the miRNA in inhibiting expression of the transgene or encoded protein.

In the context of the invention, a target sequence that binds to microRNAs expressed in the liver may be selected from SEQ ID NO's 21-28 or may be a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 21-28.

In a preferred embodiment, the target sequence of a microRNA expressed in the liver is SEQ ID NO: 21 or a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with SEQ ID NO: 21. In a further embodiment, at least one copy of a target sequence of a microRNA expressed in the liver, as described in SEQ ID NO: 21-28, is present in the gene construct of the invention. In a further embodiment, two, three, four, five, six, seven or eight copies of a target sequence of a microRNA expressed in the liver, as described in SEQ ID NO: 21-28, are present in the gene construct of the invention. In a preferred embodiment, one, two, three, four, five, six, seven or eight copies of the sequence miRT-122a (SEQ ID NO: 21) are present in the gene construct of the invention. A preferred number of copies of a target sequence of a microRNA expressed in the liver is four.

A target sequence of a microRNA expressed in the liver as used herein exerts at least a detectable level of activity of a target sequence of a microRNA expressed in the liver as known to a person of skill in the art. An activity of a target sequence of a microRNA expressed in the liver is to bind to its cognate microRNA expressed in the liver and, when operatively linked to a transgene, to mediate detargeting of transgene expression in the liver. This activity may be assessed by measuring the levels of transgene expression in the liver on the level of the mRNA or the protein by standard assays known to a person of skill in the art, such as qPCR, Western blot analysis or ELISA.

In the context of the invention, a target sequence of a microRNA expressed in the heart may be selected from SEQ ID NO's: 29-34 or may be a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 29-34 In a preferred embodiment, the target sequence of a microRNA expressed in the heart is SEQ ID NO: 29 or may be a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 29. In a further embodiment, at least one copy of a target sequence of a microRNA expressed in the heart, as described in SEQ ID NO: 29-34, is present in the gene construct of the invention. In a further embodiment, two, three, four, five, six, seven or eight copies of a target sequence of a microRNA expressed in the heart, as described in SEQ ID NO: 29-34, are present in the gene construct of the invention. In a preferred embodiment, one, two, three, four, five, six, seven or eight copies of a nucleotide sequence encoding miRT-1 (SEQ ID NO: 29), are present in the gene construct of the invention. A preferred number of copies of a target sequence of a microRNA expressed in the heart is four.

A target sequence of a microRNA expressed in the heart as used herein exerts at least a detectable level of activity of a target sequence of a microRNA expressed in the heart as known to a person of skill in the art. An activity of a target sequence of a microRNA expressed in the heart is to bind to its cognate microRNA expressed in the heart and, when operatively linked to a transgene, to mediate detargeting of transgene expression in the heart. This activity may be assessed by measuring the levels of transgene expression in the heart on the level of the mRNA or the protein by standard assays known to a person of skill in the art, such as qPCR, Western blot analysis or ELISA.

In some embodiments, at least one copy of a target sequence of a microRNA expressed in the liver, as described in SEQ ID NO: 21-28, and at least one copy of a target sequence of a microRNA expressed in the heart, as described in SEQ ID NO: 29-34, are present in the gene construct of the invention. In a further embodiment, two, three, four, five, six, seven or eight copies of a target sequence of a microRNA expressed in the liver, as described in SEQ ID NO: 29-34, and two, three, four, five, six, seven or eight copies of a target sequence of a microRNA expressed in the heart, as described in SEQ ID NO: 29-34, are present in the gene construct of the invention. In a further embodiment one, two, three, four, five, six, seven or eight copies of a nucleotide sequence encoding miRT-122a (SEQ ID NO: 21) and one, two, three, four, five, six, seven or eight copies nucleotide sequence encoding miRT-1 (SEQ ID NO: 29) are combined in the gene construct of the invention. In a further embodiment, four copies of a nucleotide sequence encoding miRT-122a (SEQ ID NO: 21) and four copies of nucleotide sequence encoding miRT-1 (SEQ ID NO: 29) are combined in the gene construct of the invention.

In some embodiments there is provided a gene construct as described above, wherein the target sequence of a microRNA expressed in the liver and the target sequence of a microRNA expressed in the heart is selected from a group consisting of sequences SEQ ID NO: 21-34 and/or combinations thereof. In some embodiments there is provided a gene construct as described above, wherein the target sequence of a microRNA expressed in the heart is selected from SEQ ID NO's: 29-34 and a target sequence of a microRNA expressed in the liver is selected from SEQ ID NO's: 21-28. In some embodiments there is provided a gene construct as described above, wherein the gene construct comprises a target sequence of microRNA-122a (SEQ ID NO: 21) and a target sequence of microRNA-1 (SEQ ID NO: 29).

In some embodiments, a ubiquitous promoter as described herein is selected from the group consisting of a CAG promoter, a CMV promoter, a mini-CMV promoter, a β-actin promoter, a rous-sarcoma-virus (RSV) promoter, an elongation factor 1 alpha (EF1a) promoter, an early growth response factor-1 (Egr-1) promoter, an Eukaryotic Initiation Factor 4A (eIF4A) promoter, a ferritin heavy chain-encoding gene (FerH) promoter, a ferritin heavy light-encoding gene (FerL) promoter, a glyceraldehyde-3-phosphate dehydrogenase (GAPDH) promoter, a GRP78 promoter, a GRP94 promoter, a heat-shock protein 70 (hsp70) promoter, an ubiquitin B promoter, a SV40 promoter, a Beta-Kinesin promoter, a ROSA26 promoter and a PGK-1 promoter. In some embodiments, a ubiquitous promoter as described herein is selected from the group consisting of a β-actin promoter, a rous-sarcoma-virus (RSV) promoter, an elongation factor 1 alpha (EF1a) promoter, an early growth response factor-1 (Egr-1) promoter, an Eukaryotic Initiation Factor 4A (eIF4A) promoter, a ferritin heavy chain-encoding gene (FerH) promoter, a ferritin heavy light-encoding gene (FerL) promoter, a glyceraldehyde-3-phosphate dehydrogenase (GAPDH) promoter, a GRP78 promoter, a GRP94 promoter, a heat-shock protein 70 (hsp70) promoter, an ubiquitin B promoter, a SV40 promoter, a Beta-Kinesin promoter, a ROSA26 promoter and a PGK-1 promoter. In preferred embodiments, a ubiquitous promoter as described herein is selected from the group consisting of a CAG promoter, a CMV promoter and a mini-CMV promoter, preferably from the group consisting of a CAG promoter and a CMV promoter, more preferably a CAG promoter.

In a preferred embodiment, the ubiquitous promoter is a CAG promoter. In some embodiments, a CAG promoter comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 35.

Another preferred ubiquitous promoter is a cytomegalovirus (CMV) promoter. In some embodiments, a CMV promoter comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 36. Preferably said CMV promoter is used together with an intronic sequence. In some embodiments, an intronic sequence comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 37.

Another preferred ubiquitous promoter is a mini-CMV promoter. In some embodiments, a mini-CMV promoter comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 38.

Another preferred ubiquitous promoter is an EF1α promoter. In some embodiments, an EF1α promoter comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 39.

Another preferred ubiquitous promoter is an RSV promoter. In some embodiments, an RSV promoter comprises, consists essentially of, or consists of a nucleotide sequence that has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity with SEQ ID NO: 40.

Additional sequences may be present in a gene construct as described herein. Exemplary additional sequences suitable herein include inverted terminal repeats (ITRs), an SV40 polyadenylation signal (SEQ ID NO: 41), a rabbit beta-globin polyadenylation signal (SEQ ID NO: 42), a CMV enhancer sequence (SEQ ID NO: 43) and a chimeric intron composed of introns from human beta-globin and immunoglobulin heavy chain genes (SEQ ID NO: 37). Within the context of the invention, “ITRs” is intended to encompass one 5′ITR and one 3′ITR, each being derived from the genome of an AAV.

Preferred ITRs are from AAV2 and are represented by SEQ ID NO: 44 (5′ ITR) and SEQ ID NO: 45 (3′ ITR). Within the context of the invention, it is encompassed to use the CMV enhancer sequence (SEQ ID NO: 43) and the CMV promoter sequence (SEQ ID NO: 36) as two separate sequences or as a single sequence (SEQ ID NO: 46). Each of these additional sequences may be present in a gene construct according to the invention. In some embodiments, there is provided a gene construct comprising a nucleotide sequence encoding HNF, preferably HNF1A, as described herein, further comprising one 5′ITR and one 3′ITR, preferably AAV2 ITRs, more preferably the AAV2 ITRs represented by SEQ ID NO: 44 (5′ ITR) and SEQ ID NO: 45 (3′ ITR). In some embodiments, there is provided a gene construct comprising a nucleotide sequence encoding an HNF, preferably an HNF1A, more preferably an HNF1A isoform a, as described herein, further comprising a polyadenylation signal, preferably an SV40 polyadenylation signal (preferably represented by SEQ ID NO: 41) and/or a rabbit β-globin polyadenylation signal (preferably represented by SEQ ID NO: 42).

Optionally, additional nucleotide sequences may be operably linked to the nucleotide sequence(s) encoding an HNF, preferably an HNF1A, more preferably an HNF1A isoform a, such as nucleotide sequences encoding signal sequences, nuclear localization signals, expression enhancers, and the like.

In some embodiments the gene construct comprises a nucleotide sequence encoding an HNF1A, preferably an HNF1A isoform a, operably linked to a RIPI promoter or a derivative thereof. Optionally, the gene construct further includes 5′ and 3′ flanks of inverted terminal repeats (ITRs) derived from the genome of an AAV, preferably from AAV2. In some embodiments, such gene construct has the nucleotide sequence of SEQ ID NO: 47, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In some embodiments the gene construct comprises a nucleotide sequence encoding an HNF1A, preferably an HNF1A isoform a, operably linked to a RIPII promoter or a derivative thereof. Optionally, the gene construct further includes 5′ and 3′ flanks of inverted terminal repeats (ITRs) derived from the genome of an AAV, preferably from AAV2. In some embodiments, such gene construct has the nucleotide sequence of SEQ ID NO: 48, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In some embodiments the gene construct comprises a nucleotide sequence encoding an HNF1A, preferably an HNF1A Isoform a, operably linked to the full-length human insulin promoter (hlNS1.9) or a derivative thereof. Optionally, the gene construct further includes 5′ and 3′ flanks of inverted terminal repeats (ITRs) derived from the genome of an AAV, preferably from AAV2. In some embodiments, such gene construct has the nucleotide sequence of SEQ ID NO: 49, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In preferred embodiments the gene construct comprises a nucleotide sequence encoding an HNF1A, preferably an HNF1A isoform a, operably linked to the 385 bp fragment of the human insulin promoter described elsewhere herein (hlns385, SEQ ID NO: 20) or a derivative thereof. Optionally, the gene construct further includes 5′ and 3′ flanks of inverted terminal repeats (ITRs) derived from the genome of an AAV, preferably from AAV2. In some embodiments, such gene construct has the nucleotide sequence of SEQ ID NO: 50, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

For any sequence described herein, in some embodiments, the level of sequence identity or similarity as used herein is preferably 70%. Another preferred level of sequence identity or similarity is 80%. Another preferred level of sequence identity or similarity is 90%. Another preferred level of sequence identity or similarity is 95%. Another preferred level of sequence identity or similarity is 99%.

### Expression Vector

Gene constructs described herein can be placed in expression vectors. Thus, in another aspect there is provided an expression vector comprising a gene construct as described in any of the preceding embodiments.

A description of “expression vector” has been provided under the section entitled “general information”. The skilled person understands that the term “expression vector” includes non-viral and viral vectors. Suitable expression vectors may be selected from any genetic element which can facilitate transfer of genes or nucleic acids between cells, such as, but not limited to, a plasmid, phage, transposon, cosmid, chromosome, artificial chromosome, virus, virion, etc. A suitable expression vector may also be a chemical vector, such as a lipid complex or naked DNA. “Naked DNA” or “naked nucleic acid” refers to a nucleic acid molecule that is not contained within a viral particle, bacterial cell, or other encapsulating means that facilitates delivery of nucleic acid into the cytoplasm of the target cell. Optionally, a naked nucleic acid can be associated with standard means used in the art for facilitating its delivery of the nucleic acid to the target cell, for example to facilitate the transport of the nucleic acid through the alimentary canal, to protect the nucleic acid from stomach acid and/or nucleases, and/or serve to penetrate intestinal mucus.

In a preferred embodiment, the expression vector is a viral expression vector. A description of “viral expression vector” has been provided under the section entitled “general information”.

A viral vector may be a viral vector selected from the group consisting of adenoviral vectors, adeno-associated viral vectors, retroviral vectors and lentiviral vectors. An adenoviral vector is also known as an adenovirus derived vector, an adeno-associated viral vector is also known as an adeno-associated virus derived vector, a retroviral vector is also known as a retrovirus derived vector and a lentiviral vector is also known as a lentivirus derived vector. A preferred viral vector is an adeno-associated viral vector. A description of “adeno-associated viral vector” has been provided under the section entitled “general information”.

In some embodiments, the vector is an adeno-associated vector or adeno-associated viral vector or an adeno-associated virus derived vector (AAV) selected from the group consisting of AAV of serotype 1 (AAV1), AAV of serotype 2 (AAV2), AAV of serotype 3 (AAV3), AAV of serotype 4 (AAV4), AAV of serotype 5 (AAV5), AAV of serotype 6 (AAV6), AAV of serotype 7 (AAV7), AAV of serotype 8 (AAV8), AAV of serotype 9 (AAV9), AAV of serotype rh10 (AAVrh10), AAV of serotype rh8 (AAVrh8), AAV of serotype Cb4 (AAVCb4), AAV of serotype rh74 (AAVrh74), AAV of serotype DJ (AAVDJ), AAV of serotype 2/5 (AAV2/5), AAV of serotype 2/1 (AAV2/1), AAV of serotype 1/2 (AAV1/2) and AAV of serotype Anc80 (AAVAnc80).

In a preferred embodiment, the vector is an AAV of serotype 6, 8 or 9 (AAV6, AAV8, or AAV9). In a more preferred embodiment, the vector is an AAV of serotype 6 or 8 (AAV6 or AAV8), preferably it is AAV8.

In some embodiments the expression vector is an AAV8 and comprises a gene construct comprising a nucleotide sequence encoding an HNF1A, preferably an HNF1A isoform a, operably linked to a RIPI promoter or a derivative thereof. Optionally, the gene construct further includes 5′ and 3′ flanks of inverted terminal repeats (ITRs) derived from the genome of an AAV, preferably from AAV2. In some embodiments, such expression vector comprises a gene construct having the nucleotide sequence of SEQ ID NO: 47, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In some embodiments the expression vector is an AAV8 and comprises a gene construct comprising a nucleotide sequence encoding an HNF1A, preferably an HNF1A isoform a, operably linked to a RIPII promoter or a derivative thereof. Optionally, the gene construct further includes 5′ and 3′ flanks of inverted terminal repeats (ITRs) derived from the genome of an AAV, preferably from AAV2. In some embodiments, such expression vector comprises a gene construct having the nucleotide sequence of SEQ ID NO: 48, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In some embodiments the expression vector is an AAV8 and comprises a gene construct comprising a nucleotide sequence encoding an HNF1A, preferably an HNF1A Isoform a, operably linked to the full-length human insulin promoter (hlNS1.9) or a derivative thereof. Optionally, the gene construct further includes 5′ and 3′ flanks of inverted terminal repeats (ITRs) derived from the genome of an AAV, preferably from AAV2. In some embodiments, such expression vector comprises a gene construct having the nucleotide sequence of SEQ ID NO: 49, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

In preferred embodiments the expression vector is an AAV8 and comprises a gene construct comprising a nucleotide sequence encoding an HNF1A, preferably an HNF1A isoform a, operably linked to the 385 bp fragment of the human insulin promoter described elsewhere herein (hlns385, SEQ ID NO: 20) or a derivative thereof. Optionally, the gene construct further includes 5′ and 3′ flanks of inverted terminal repeats (ITRs) derived from the genome of an AAV, preferably from AAV2. In some embodiments, such expression vector comprises a gene construct having the nucleotide sequence of SEQ ID NO: 50, or a sequence having at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity therewith.

The production of recombinant AAV (rAAV) for vectorizing transgenes have been described previously. See Ayuso E, et al., Curr. Gene Ther. 2010; 10:423-436, Okada T, et al., Hum. Gene Ther. 2009; 20:1013-1021, Zhang H, et al., Hum. Gene Ther. 2009; 20:922-929, and Virag T, et al., Hum. Gene Ther. 2009; 20:807-817; all of which incorporated herein by reference. These protocols can be used or adapted to generate the AAV of the invention. Thus, in another aspect there is provided a method for producing an adeno-associated viral vector as described herein.

In short, the methods generally involve (a) the introduction of the AAV genome comprising the gene construct to be expressed into a cell, (b) the presence or introduction of an AAV helper construct in the cell, wherein the helper construct comprises the viral functions missing from the AAV genome and, optionally, (c) the introduction of a helper virus into the host cell. All components for AAV vector replication and packaging need to be present, to achieve replication and packaging of the AAV genome into AAV vectors. These typically include AAV cap proteins, AAV rep proteins and, optionally, viral proteins upon which AAV is dependent for replication. Rep and cap regions are well known in the art, see e.g. Chiorini et al. (1999, J. of Virology, Vol 73(2): 1309-1319, incorporated herein by reference) or U.S. Pat. No. 5,139,941 (incorporated herein by reference). The AAV cap and rep proteins may derive from the same AAV serotype or they can derive from a combination of different serotypes, preferably they derive from an AAV8 serotype. The viral proteins upon which AAV is dependent for replication may derive from any virus, such as a herpes simplex viruses (such as HSV types 1 and 2), a vaccinia virus, an adeno-associated virus or an adenovirus, preferably from an adenovirus.

In some embodiments, the producer cell line is transfected transiently with the polynucleotide of the invention (comprising the expression cassette flanked by ITRs) and with construct(s) that encode(s) rep and cap proteins and provide(s) helper functions. In some embodiments, the cell line supplies stably the helper functions and is transfected transiently with the polynucleotide of the invention (comprising the expression cassette flanked by ITRs) and with construct(s) that encode(s) rep and cap proteins. In some embodiments, the cell line supplies stably the rep and cap proteins and the helper functions and is transiently transfected with the polynucleotide of the invention. In another embodiment, the cell line supplies stably the rep and cap proteins and is transfected transiently with the polynucleotide of the invention and a polynucleotide encoding the helper functions. In some embodiments, the cell line supplies stably the polynucleotide of the invention, the rep and cap proteins and the helper functions. Methods of making and using these and other AAV production systems have been described in the art. See Muzyczka N, et al., U.S. Pat. No. 5,139,941, Zhou X, et al., U.S. Pat. No. 5,741,683, Samulski R, et al., U.S. Pat. No. 6,057,152, Samulski R, et al., U.S. Pat. No. 6,204,059, Samulski R, et al., U.S. Pat. No. 6,268,213, Rabinowitz J, et al., U.S. Pat. No. 6,491,907, Zolotukhin S, et al., U.S. Pat. No. 6,660,514, Shenk T, et al., U.S. Pat. No. 6,951,753, Snyder R, et al., U.S. Pat. No. 7,094,604, Rabinowitz J, et al., U.S. Pat. No. 7,172,893, Monahan P, et al., U.S. Pat. No. 7,201,898, Samulski R, et al., U.S. Pat. No. 7,229,823, and Ferrari F, et al., U.S. Pat. No. 7,439,065, all of which are incorporated herein by reference.

The recombinant AAV (rAAV) genome present in a rAAV vector comprises at least the nucleotide sequences of the inverted terminal repeat regions (ITRs) of one of the AAV serotypes (preferably the ones of serotype AAV2 as disclosed herein), or nucleotide sequences substantially identical thereto or nucleotide sequences having at least 60%, 70%, 80%, 90%, 95% or 99% identity thereto, and nucleotide sequence encoding an HNF, preferably an HNF1A, more preferably an HNF1A isoform a, (under control of a suitable regulatory element) inserted between the two ITRs. A vector genome generally requires the use of flanking 5′ and a 3′ ITR sequences to allow for efficient packaging of the vector genome into the rAAV capsid.

The complete genome of several AAV serotypes and corresponding ITRs has been sequenced (Chiorini et al. 1999, J. of Virology Vol. 73, No. 2, p 1309-1319, incorporated herein by reference). They can be either cloned or made by chemical synthesis as known in the art, using for example an oligonucleotide synthesizer as supplied e.g. by Applied Biosystems Inc. (Fosters, CA, USA) or by standard molecular biology techniques. The ITRs can be cloned from the AAV viral genome or excised from a vector comprising the AAV ITRs. The ITR nucleotide sequences can be either ligated at either end to the nucleotide sequence comprising one or more genes using standard molecular biology techniques, or the AAV sequence between the ITRs can be replaced with the desired nucleotide sequence.

Preferably, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

The rAAV genome as present in said rAAV vector further comprises a promoter sequence operably linked to the nucleotide sequence encoding an HNF, preferably an HNF1A, more preferably an HNF1A isoform a.

A suitable 3′ untranslated sequence may also be operably linked to the nucleotide sequence encoding an HNF, preferably an HNF1A, more preferably an HNF1A isoform a. Suitable 3′ untranslated regions may be those naturally associated with the nucleotide sequence or may be derived from different genes, such as for example the SV40 polyadenylation signal (SEQ ID NO: 49) and the rabbit β-globin polyadenylation signal (SEQ ID NO: 50).

The introduction into a producer cell can be carried out using standard virological techniques, such as transformation, transduction and transfection. Most vectors do not replicate in the producer cells infected with the vector. Examples of workable combinations of cell lines and expression vectors are described in Sambrook and Green, Molecular Cloning. A Laboratory Manual, 4^{th} Edition (2012), Cold Spring Harbor Laboratory Press (incorporated herein by reference), and in Metzger et al (1988) Nature 334: 31-36 (incorporated herein by reference). For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic producer cell. A cell may be a cell that is suitable for culture in liquid or on solid media. Finally, the producere cells are cultured under standard conditions known in the art to produce the assembled AAV vectors which are then purified using standard techniques such as polyethylene glycol precipitation or CsCl gradients (Xiao et al. 1996, J. Virol. 70: 8098-8108, incorporated herein by reference). Residual helper virus activity can be inactivated using known methods, such as for example heat inactivation.

The gene constructs and expression vectors as described herein may then be introduced into a host cell using standard molecular techniques, as discussed in standard handbooks such as Current Protocols in Molecular Biology (Ausubel et al.), 3^{rd} edition (2003), John Wiley & Sons, Inc (US) (incorporated herein by reference) and Sambrook and Green (2012, supra). Accordingly, the invention further provides a host cell transduced with any of the gene constructs or expression vectors described herein. In some embodiments, a host cell transduced with any of the gene constructs or expression vectors described herein is a pancreatic cell, such as a pancreatic cell of a vertebrate, preferably a pancreatic cell of a mammal. In preferred embodiments, a host cell transduced with any of the gene constructs or expression vectors described herein is a pancreatic cell of a rat, mouse, dog or a human, preferably of a mouse or a human, more preferably a human.

In some embodiments, a pancreatic cell as described herein is a pancreatic islet cell, more preferably a beta cell.

In the case of viral vectors, transduction is preferably used. The transduced host cell may or may not comprise the packaging components of the viral vectors. “Host cell” or “target cell” refers to the cell into which the DNA delivery takes place, such as the pancreatic cells of a mammalian subject as described elsewhere herein. AAV vectors in particular are able to transduce both dividing and non-dividing cells.

The provided pancreatic and/or pancreatic islet and/or beta cell host cells need not necessarily be present in an individual. The skilled person understands that introduction of the gene constructs and expression vectors as described herein may be performed in cell cultures. In some embodiments, the provided pancreatic and/or pancreatic islet and/or beta cell host cells are present in an artificial organ, preferably an artificial pancreas. In some embodiments, the provided pancreatic and/or pancreatic islet and/or beta cell host cells are present in an organoid, preferably a pancreas organoid. An “organoid” as defined herein is a miniaturized and simplified version of an organ produced in vitro in three dimensions that shows realistic micro-anatomy. The skilled person is able to arrive at such artificial organs and/or organoids using the host cells of the invention by applying generally known procedures in the art. The transduced host cells present in an artificial organ and/or organoid may be implanted to a vertebrate, preferably a mammal, more preferably a mouse, rat, dog or human, more preferably a mouse or human, most preferably a human, using generally known procedures in the art.

### Composition

In a further aspect there is provided a composition comprising a gene construct as described above and/or an expression vector as described above, optionally further comprising one or more pharmaceutically acceptable ingredients. Such composition may be called a gene therapy composition. Preferably, the composition is a pharmaceutical composition.

As used herein, “pharmaceutically acceptable ingredients” include pharmaceutically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents and/or excipients. Such pharmaceutically acceptable carriers, fillers, preservatives, solubilizers, vehicles, diluents and/or excipients may for instance be found in Remington: The Science and Practice of Pharmacy, 23rd edition. Elsevier (2020), incorporated herein by reference.

A further compound may be present in a composition of the invention. Said compound may help in delivery of the composition. Suitable compounds in this context are: compounds capable of forming complexes, nanoparticles, micelles and/or liposomes that deliver each constituent as described herein, complexed or trapped in a vesicle or liposome through a cell membrane. Many of these compounds are known in the art. Suitable compounds comprise polyethylenimine (PEI), or similar cationic polymers, including polypropyleneimine or polyethylenimine copolymers (PECs) and derivatives; synthetic amphiphiles (SAINT-18); Lipofectin™, DOTAP. A person of skill in the art will know which type of formulation is the most appropriate for a composition as described herein.

### Method and Use

Also provided herein are gene constructs, expression vectors and compositions as described herein for use in therapy. In some embodiments, gene constructs, expression vectors and compositions as described herein are for use as a medicament.

In preferred embodiments, gene constructs, expression vectors, and compositions as described herein are provided for use in the treatment and/or prevention of a maturity-onset diabetes of the young (MODY) or a condition associated therewith, preferably MODY3 or a condition associated therewith, as described elsewhere herein. MODY3 is a MODY which is associated with mutations of HNF1A. Accordingly, in preferred embodiments, gene constructs, expression vectors, and compositions as described herein are provided for use in the treatment and/or prevention of a maturity-onset diabetes of the young which is MODY3.

In a further aspect there is provided a method of treatment and/or prevention of a maturity-onset diabetes of the young (MODY) or a condition associated therewith, preferably MODY3 or a condition associated therewith, comprising administering a gene construct, an expression vector and/or a composition as described herein. In some embodiments, administering a gene construct, an expression vector or a composition means administering to a subject such as a subject in need thereof. In a preferred embodiment, a therapeutically effective amount of a gene construct, an expression vector or a composition is administered.

As used herein, an “effective amount” is an amount sufficient to exert beneficial or desired results. Accordingly, a “therapeutically effective amount” is an amount that, when administered to a subject in need thereof, is sufficient to exert some therapeutic effect as described herein, such as, but not limited to, a reduction in hyperglycemia and an increase in glucose tolerance compared to an untreated subject. An amount that is “therapeutically effective” will vary from subject to subject, depending on the age, the disease progression and overall general condition of the individual. An appropriate “therapeutically effective” amount in any individual case may be determined by the skilled person using routine experimentation, such as the methods described later herein, and/or the methods of the experimental part herein.

In a further aspect there is provided a use of a gene construct, an expression vector or a composition as described herein, for the manufacture of a medicament for the treatment and/or prevention of a maturity-onset diabetes of the young (MODY) or a condition associated therewith, preferably MODY3 or a condition associated therewith.

In a further aspect there is provided a use of a gene construct, an expression vector or a composition as described herein, for the treatment and/or prevention of a maturity-onset diabetes of the young (MODY) or a condition associated therewith, preferably MODY3 or a condition associated therewith.

Within the context of gene constructs for use, expression vectors for use, compositions for use, methods and uses according to the invention, the therapy and/or treatment and/or medicament may involve expression of HNF, preferably an HNF1A, more preferably an HNF1A isoform a, in the pancreas and/or transduction of the pancreas. In some embodiments, expression of HNF, preferably an HNF1A, more preferably an HNF1A isoform a, in the pancreas may mean expression of said HNF in the pancreatic islets and/or beta-cells. In some embodiments, expression in and/or transduction of the pancreas and/or the pancreatic islets and/or the beta-cells may mean specific expression in and/or specific transduction of the pancreas and/or the pancreatic islets and/or the beta-cells. In an embodiment, expression does not involve expression in the CNS, liver, brain, adipose tissue, skeletal muscle and/or heart, preferably in the liver and/or heart. In some embodiments, expression does not involve expression in at least one, at least two, at least three, at least four or all organs selected from the group consisting of the CNS, liver, brain, adipose tissue, skeletal muscle and heart, preferably selected from the liver and heart. A description of pancreas-, pancreatic islet-, and beta-cell-specific expression has been provided under the section entitled “general information”.

Within the context of gene constructs for use, expression vectors for use, compositions for use, methods and uses according to the invention, “involving the expression of a gene construct” may be replaced by “causing the expression of a gene construct” or “inducing the expression of a gene construct” or “involving transduction”.

In a preferred embodiment, a treatment or a therapy or a use or the administration of a medicament as described herein does not have to be repeated. In some embodiments, a treatment or a therapy or a use or the administration of a medicament as described herein may be repeated each year or each 2, 3, 4, 5, 6, 7, 8, 9 or 10, including intervals between any two of the listed values, years.

The subject treated may be a vertebrate, preferably a mammal, such as a cat, a rodent (preferably mice, rats), a dog, or a human. In preferred embodiments, the subject treated is a human.

Within the context of gene constructs for use, expression vectors for use, compositions for use, methods and uses according to the invention, a gene construct and/or an expression vector and/or a composition and/or a medicament as described herein preferably exhibits at least one, at least two, at least three, or all of the following effects:
increase of beta-cell mass;
restoration of beta-cell function;
alleviating a symptom of MODY, preferably MODY3 (as described herein); and
improving a parameter associated with MODY, preferably MODY3 (as described herein).

In some embodiments, a gene construct and/or an expression vector and/or a composition and/or a medicament as described herein preferably exhibits at least one, at least two, at least three, or all of the following effects:
decreased hyperglycemia;
increased glucose tolerance; and
maintenance of body weight

Alleviating a symptom of MODY may mean that a symptom of MODY (e.g. the onset of hyperglycemia and a decrease in glucose tolerance) is improved or decreased or that the progression of a typical symptom has been slowed down in an individual, in a cell, tissue or organ of said individual as assessed by a physician. A decrease or improvement of a typical symptom may mean a slowdown in progression of symptom development or a complete disappearance of symptoms. Symptoms, and thus also a decrease in symptoms, can be assessed using a variety of methods, to a large extent the same methods as used in diagnosis of MODY, including clinical examination and routine laboratory tests. Laboratory tests may include both macroscopic and microscopic methods, molecular methods, radiographic methods such as X-rays, biochemical methods, immunohistochemical methods and others. Hyperglycemia and glucose tolerance could be assessed using techniques known to a person of skill in the art, for example as done in the experimental part. An exemplary marker that could be used in this regard is the blood glucose level. In this context, “decrease” (respectively “improvement”) means at least a detectable decrease (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The decrease may be seen after at least one week, one month, six months, one year or more of treatment using a gene construct and/or an expression vector and/or a composition of the invention. Preferably, the decrease is observed after a single administration. In some embodiments, the decrease is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Improving a parameter may mean that the value of a typical parameter associated with MODY (e.g. hyperglycemia and decreased glucose tolerance) is improved in an individual, in a cell, tissue or organ of said individual as assessed by a physician. In this context, improvement of a parameter may be interpreted as to mean that said parameter assumes a value closer to the value displayed by a healthy individual. The improvement of a parameter may be seen after at least one week, one month, six months, one year or more of treatment using a gene construct and/or an expression vector and/or a composition of the invention. Preferably, the improvement is observed after a single administration. In some embodiments, the improvement is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

A gene construct and/or an expression vector and/or a composition as described herein is preferably able to alleviate a symptom or a parameter or a characteristic of MODY, preferably MODY3, in a patient or of a cell, tissue or organ of said patient if after at least one week, one month, six months, one year or more of treatment using a gene construct and/or an expression vector and/or a composition of the invention, said symptom or characteristic has decreased (e.g. is no longer detectable or has slowed down), as described herein.

A gene construct and/or an expression vector and/or a composition as described herein may be suitable for administration to a cell, tissue and/or an organ in vivo of individuals affected by or at risk of developing MODY, preferably MODY3, and may be administered in vivo, ex vivo or in vitro. Said gene construct and/or expression vector and/or composition may be directly or indirectly administered to a cell, tissue and/or an organ in vivo of an individual affected by or at risk of developing MODY, preferably MODY3, and may be administered directly or indirectly in vivo, ex vivo or in vitro.

Within the context of gene constructs for use, expression vectors for use, compositions for use, methods and uses according to the invention, a gene construct and/or an expression vector and/or a composition may be administered by different administration modes. An administration mode may be intravenous, intramuscular, intraperitoneal, via inhalation, intraparenchymal, subcutaneous, intraarticular, intra-adipose tissue, oral, intrahepatic, intrasplanchnic, intra-ear, and/or via intraductal administration. A preferred administration mode is intraductal administration, preferably pancreatic intraductal administration. “Intraductal administration” refers to administration within the duct of a gland.

A gene construct and/or an expression vector and/or a composition of the invention may be directly or indirectly administered using suitable means known in the art. Improvements in means for providing an individual or a cell, tissue, organ of said individual with a gene construct and/or an expression vector and/or a composition of the invention are anticipated, considering the progress that has already thus far been achieved. Such future improvements may of course be incorporated to achieve the mentioned effect of the invention. A gene construct and/or an expression vector and/or a composition can be delivered as is to an individual, a cell, tissue or organ of said individual. Depending on the disease or condition, a cell, tissue or organ of said individual may be as earlier described herein. When administering a gene construct and/or an expression vector and/or a composition of the invention, it is preferred that such gene construct and/or an expression vector and/or a composition is dissolved in a solution that is compatible with the delivery method.

As encompassed herein, a therapeutically effective dose of a gene construct and/or an expression vector and/or a composition as mentioned above is preferably administered in a single and unique dose hence avoiding repeated periodical administration.

### General Information

Unless stated otherwise, all technical and scientific terms used herein have the same meaning as customarily and ordinarily understood by a person of ordinary skill in the art to which this invention belongs, and read in view of this disclosure.

### Pancreas

The term “pancreas” as used herein refers the organ of the digestive system and endocrine system of vertebrates as customarily and ordinarily understood by the skilled person. “Pancreatic islets”, also known as “pancreatic islands” or “islets of Langerhans” refer to the regions of the pancreas that contain its endocrine (hormone-producing) cells as as customarily and ordinarily understood by the skilled person. Pancreatic islets typically comprise alpha-cells, producing glucagon, beta-cells, producing insulin and amylin, delta-cells, producing somatostatin, epsilon-cells, producing ghrelin and PP cells (gamma-cells or F-cells), producing pancreatic polypeptide. Beta-cells are of particular importance for maintenance of blood sugar homeostasis.

### Sequence Identity

In the context of the invention, a nucleic acid molecule such as a nucleic acid molecule encoding an HNF, preferably an HNF1A, more preferably an HNF1A isoform a, is represented by a nucleic acid or nucleotide sequence which encodes a protein fragment or a polypeptide or a peptide or a derived peptide. In the context of the invention, an HNF, preferably an HNF1A, more preferably an HNF1A isoform a, protein fragment or a polypeptide or a peptide or a derived peptide is represented by an amino acid sequence.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60%, 70%, 80%, 90%, 95% or 99% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60%, 70%, 80%, 90%, 95% or 99% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60%, 70%, 80%, 90%, 95% or 99% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference).

Alternatively percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/.

Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lIle or Val; Lys to Arg; Gln or Glu; Met to Leu or lie; Phe to Met, Leu or Tyr; Ser to Thr; Thr to Ser; Trp to Tyr; Tyr to Trp or Phe; and, Val to lie or Leu.

### Gene or Coding Sequence

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site. A chimeric or recombinant gene (such as an HNF gene) is a gene not normally found in nature, such as a gene in which for example the promoter is not associated in nature with part or all of the transcribed DNA region. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

A “transgene” is herein described as a gene or a coding sequence or a nucleic acid molecule (i.e. a molecule encoding an HNF) that has been newly introduced into a cell, i.e. a gene that may be present but may normally not be expressed or expressed at an insufficient level in a cell. In this context, “insufficient” means that although said HNF is expressed in a cell, a condition and/or disease as described herein could still be developed. In this case, the invention allows the over-expression of a HNF. The transgene may comprise sequences that are native to the cell, sequences that naturally do not occur in the cell and it may comprise combinations of both. A transgene may contain sequences coding for a HNF and/or additional proteins as earlier identified herein that may be operably linked to appropriate regulatory sequences for expression of the sequences coding for a HNF in the cell. Preferably, the transgene is not integrated into the host cell's genome.

### Promoter

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

A “ubiquitous promoter” is active in substantially all tissues, organs and cells of an organism.

A “organ-specific” or “tissue-specific” promoter is a promoter that is active in a specific type of organ or tissue, respectively. Organ-specific and tissue-specific promoters regulate expression of one or more genes (or coding sequence) primarily in one organ or tissue, but can allow detectable level (“leaky”) expression in other organs or tissues as well. Leaky expression in other organs or tissues means at least one-fold, at least two-fold, at least three-fold, at least four-fold or at least five-fold lower, but still detectable expression as compared to the organ-specific or tissue-specific expression, as evaluated on the level of the mRNA or the protein by standard assays known to a person of skill in the art (e.g. qPCR, Western blot analysis, ELISA). The maximum number of organs or tissues where leaky expression may be detected is five, six, seven or eight.

Assessment of the ubiquitous or tissue-specific nature of a promoter can be performed by standard molecular toolbox techniques, such as, for example, described in Sambrook and Green (supra). As a non-limiting example, any expression vector comprising any of the gene construct as described herein, wherein the HNF nucleotide sequence has been replaced by a nucleotide sequence encoding for GFP, can be produced. Cells transduced as described herein can then be assessed for fluorescence intensity according to standard protocols.

A “pancreas-specific promoter” is a promoter that is capable of initiating transcription in the pancreas, whilst still allowing for any leaky expression in other (maximum five, six, seven or eight) organs and parts of the body. Transcription in the pancreas can be detected in relevant areas, such as the head, uncinated process, neck, body, tail, endocrine and exocrine parts. Promoters that are capable of initiating transcription in cells of the pancreatic islets (pancreatic islet-specific), preferably in alpha-cells, beta-cells, delta-cells, epsilon-cells and PP cells (gamma-cells or F-cells), whilst still allowing for any leaky expression in other (maximum five, six, seven or eight) organs and parts of the body, are advantageous. Promoters that are capable of initiating transcription in pancreatic beta-cells (beta-cell specific), whilst still allowing for any leaky expression in other (maximum five, six, seven or eight) organs and parts of the body, are particurlarly advantageous.

In the context of the invention, pancreas- and/or pancreatic islet- and/or beta-cell-specific promoters may be promoters that are capable of driving the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of an HNF, preferably an HNF1A, more preferably an HNF1A isoform a, in the pancreas and/or the pancreatic islets and/or the beta-cells as compared to other organs or tissues. Other organs or tissues may be the liver, CNS, brain, adipose tissue, skeletal muscle, heart, kidney, colon, hematopoietic tissue, lung, ovary, spleen, stomach, testis and others. Preferably, other organs are the liver and the heart.

As used herein, a “regulator” or “transcriptional regulator” is a protein that controls the rate of transcription of genetic information from DNA to messenger RNA, by binding to a specific DNA sequence. Expression may be assessed as described elsewhere under the section entitled “general information”.

### Operably Linked

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. Linking can be accomplished by ligation at convenient restriction sites or at adapters or linkers inserted in lieu thereof, or by gene synthesis.

microRNA

As used herein, “microRNA” or “miRNA” or “miR” has its customary and ordinary meaning as understood by one of skill in the art in view of this disclosure. A microRNA is a small non-coding RNA molecule found in plants, animals and some viruses, that may function in RNA silencing and post-transcriptional regulation of gene expression. A target sequence of a microRNA may be denoted as “miRT”. For example, a target sequence of microRNA-1 or miRNA-1 or miR-1 may be denoted as miRT-1.

### Proteins and Amino Acids

The terms “protein” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (lie) is isoleucine, K (Lys) is lysine, L (Leu) is leucine, M (Met) is methionine, N (Asn) is asparagine, P (Pro) is proline, Q (Gln) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

### Gene Constructs

Gene constructs as described herein could be prepared using any cloning and/or recombinant DNA techniques, as known to a person of skill in the art, in which a nucleotide sequence encoding said HNF, preferably an HNF HNF1A, more preferably an HNF1A isoform a, is expressed in a suitable cell, e.g. cultured cells or cells of a multicellular organism, such as described in Ausubel et al., “Current Protocols in Molecular Biology”, (2003, supra) and in Sambrook and Green (2012, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

### Expression Vectors

The phrase “expression vector” or “vector” or “delivery vector” generally refers to a tool in molecular biology used to obtain gene expression in a cell, for example by introducing a nucleotide sequence that is capable of effecting expression of a gene or a coding sequence in a host compatible with such sequences. An expression vector carries a genome that is able to stabilize and remain episomal in a cell. Within the context of the invention, a cell may mean to encompass a cell used to make the construct or a cell wherein the construct will be administered. Alternatively, a vector is capable of integrating into a cell's genome, for example through homologous recombination or otherwise.

These expression vectors typically include at least suitable promoter sequences and optionally, transcription termination signals. An additional factor necessary or helpful in effecting expression can also be used as described herein. A nucleic acid or DNA or nucleotide sequence encoding a HNF, preferably an HNF1A, more preferably an HNF1A isoform a, is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been described herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Green, 2012, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Green, 2012, supra). An expression vector includes the replication system and transcriptional and translational regulatory sequences together with the insertion site for the polypeptide encoding segment. In most cases, the replication system is only functional in the cell that is used to make the vector (bacterial cell as E. Coll). Most plasmids and vectors do not replicate in the cells infected with the vector. Examples of workable combinations of cell lines and expression vectors are described in Sambrook and Green (2012, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

Alternatively, a host cell is a cell that is part of a multicellular organism such as a transgenic plant or animal.

### Viral Vector

A viral vector or a viral expression vector a viral gene therapy vector is a vector that comprises a gene construct as described herein.

A viral vector or a viral gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein; all of which are incorporated herein by reference.

A particularly suitable gene therapy vector includes an adenoviral and adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications, (Russell, 2000, J. Gen. Virol. 81: 2573-2604; Goncalves, 2005, Virol J. 2(1):43; incorporated herein by reference) as indicated above. AAV vectors are even more preferred since they are known to result in very stable long-term expression of transgene expression (up to 9 years in dog (Niemeyer et al, Blood. 2009 Jan. 22; 113(4):797-806) and ˜10 years in human (Buchlis, G. et al., Blood. 2012 Mar. 29; 119(13):3038-41). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34; all of which are incorporated herein by reference.

Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285: 674-6, incorporated herein by reference). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16); all of which are incorporated herein by reference.

Other suitable gene therapy vectors include an adenovirus vector, a herpes virus vector, a polyoma virus vector or a vaccinia virus vector.

### Adeno-Associated Virus Vector (AAV Vector)

The terms “adeno associated virus”, “AAV virus”, “AAV virion”, “AAV viral particle” and “AAV particle”, used as synonyms herein, refer to a viral particle composed of at least one capsid protein of AAV (preferably composed of all capsid protein of a particular AAV serotype) and an encapsulated polynucleotide of the AAV genome. If the particle comprises a heterologous polynucleotide (i.e. a polynucleotide different from a wild-type AAV genome, such as a transgene to be delivered to a mammalian cell) flanked by AAV inverted terminal repeats, then they are typically known as a “AAV vector particle” or “AAV viral vector” or “AAV vector”. AAV refers to a virus that belongs to the genus Dependovirus family Parvoviridae. The AAV genome is approximately 4.7 Kb in length and it consists of single strand deoxyribonucleic acid (ssDNA) that can be positive or negative detected. The invention also encompasses the use of double stranded AAV also called dsAAV or scAAV. The genome includes inverted terminal repeats (ITR) at both ends of the DNA strand, and two open reading frames (ORFs): rep and cap. The frame rep is made of four overlapping genes that encode proteins Rep necessary for AAV lifecycle. The frame cap contains nucleotide sequences overlapping with capsid proteins: VP1, VP2 and VP3, which interact to form a capsid of icosahedral symmetry (see Carter and Samulski., 2000, and Gao et al, 2004, incorporated herein by reference).

A preferred viral vector or a preferred gene therapy vector is an AAV vector. An AAV vector as used herein preferably comprises a recombinant AAV vector (rAAV vector). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others. Preferred ITRs are those of AAV2 which are represented by sequences comprising, consisting essentially of, or consisting of SEQ ID NO: 44 (5′ ITR) and SEQ ID NO: 45 (3′ ITR). The invention also preferably encompasses the use of a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 44 as 5′ ITR and a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 45 as 3′ ITR.

Protein shell comprised of capsid protein may be derived from any AAV serotype. A protein shell may also be named a capsid protein shell. rAAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, at least 85%, at least 90%, at least 95%, at least 97%, at least 98%, at least 99% or 100% sequence identity with wild type sequences or may be altered by for example by insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.

A nucleic acid molecule represented by a nucleic acid sequence of choice is preferably inserted between the rAAV genome or ITR sequences as identified above, for example an expression construct comprising an expression regulatory element operably linked to a coding sequence and a 3′ termination sequence. Said nucleic acid molecule may also be called a transgene.

“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73(2): 1309-1319) or U.S. Pat. No. 5,139,941, incorporated herein by reference. The AAV helper functions can be supplied on an AAV helper construct.

Introduction of the helper construct into the host cell can occur e.g. by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand. “AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via plasmids, as described in U.S. Pat. No. 6,531,456 incorporated herein by reference.

“Transduction” refers to the delivery of an HNF into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

### Expression

Expression may be assessed by any method known to a person of skill in the art. For example, expression may be assessed by measuring the levels of transgene expression in the transduced tissue on the level of the mRNA or the protein by standard assays known to a person of skill in the art, such as qPCR, RNA sequencing, Northern blot analysis, Western blot analysis, mass spectrometry analysis of protein-derived peptides or ELISA.

Expression may be assessed at any time after administration of the gene construct, expression vector or composition as described herein. In some embodiments herein, expression may be assessed after 1 week, 2 weeks, 3 weeks, 4, weeks, 5 weeks, 6 weeks, 7 weeks, 8 weeks, 9, weeks, 10 weeks, 11 weeks, 12 weeks, 14 weeks, 16 weeks, 18 weeks, 20 weeks, 22 weeks, 24 weeks, 28 weeks, 32 weeks, 36 weeks, 40 weeks, or more.

In the context of the invention, pancreas- and/or pancreatic islet- and/or beta-cell-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of HNF, preferably an HNF1A, more preferably an HNF1A isoform a, in the pancreas and/or pancreatic islets and/or beta-cells as compared to other organs or tissues. Other organs or tissues may be the CNS, brain, liver, adipose tissue, skeletal muscle, heart, kidney, colon, hematopoietic tissue, lung, ovary, spleen, stomach, testis and others. Preferably, other organs are the liver and/or the heart. In an embodiment, expression is not detectable in the liver, CNS, brain, adipose tissue, skeletal muscle and/or heart. In some embodiments, expression is not detectable in at least one, at least two, at least three, at least four or all organs selected from the group consisting of the liver, CNS, brain, adipose tissue, skeletal muscle, heart, kidney, colon, hematopoietic tissue, lung, ovary, spleen, stomach and testis. Expression may be assessed as described above.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. For example, to suit the codon preference of mammalians, preferably of murine, canine or human expression hosts. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs).). In some embodiments, codon-optimized sequences show at least 3%, 5%, 10%, 15%, 20%, 25%, 30%, 40%, 50%, 60%, 70%, 80%, 90%, 100% or more increase in gene expression, transcription, RNA stability and/or translation compared to the original, not codon-optimized sequence.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Generation of a MODY3 mouse model. (A) CRISPR/Cas9 strategy to generate MODY3 knock-in (KI) mice. Single guided RNA (sgRNA) was designed to target between exon 10 and 3′UTR of HNF1 a gene to introduce two copies of microRNA 375 target sequence (miRT375), contained in donor DNA, by homology directed repair (HDR). Resultant knock-in allel is represented (down). (B) Genotyping of offspring by PCR and subsequent digestion of the PCR amplicon with EcoRV. ND, not digested; WT, wild-type; KI, miRT375 knock-in.

FIG. 2. Downregulation of HNF1A expression levels in islets of MODY3 mice. Gene expression in islets from 14-16-week-old WT/WT (wild-type) and KI/KI (homozygous miRT375 knock-in) (MODY3) mice. Relative expression of Hnf1a (Hepatocyte Nuclear Factor 1-Alpha) in (A) male and (B) female mice. Results are expressed as the mean±SEM. n=5-9. **p<0.01, ***p<0.001 vs WT/WT.

FIG. 3. Downregulation of HNF1A production in islets of MODY3 mice. Western-blot analysis of HNF1a protein from islets. A cohort of WT/WT (wild-type), WT/KI (heterozygous) and KI/KI (homozygous) animals were analyzed at 14-16 weeks of age. (A) A representative immunoblot of HNF1 a protein and normalizer tubulin protein is shown. The graphs showed the densiometric analysis of males (B) and females (C) mice. Results are expressed as the mean±SEM. n=3-5.

FIG. 4. MODY3 mice presented similar HNF1A production in liver than wild type mice. Western-blot analysis of HNF1a protein from liver of male (A) and female (B) mice. A cohort of WT/WT (wild-type), WT/KI (heterozygous) and KI/KI (homozygous) animals were analyzed at 14-16 weeks of age. A representative immunoblot is shown up). The graph shows the densiometric analysis of two different immunoblots (down). Results are expressed as the mean±SEM. n=3-6.

FIG. 5. MODY3 Knock-in mice did not exhibit changes in body weight. Body weight evolution of WT/WT (wild-type), WT/KI (heterozygous) and KI/KI (homozygous) from 4 to 14 weeks of age in male (A) and female (B) mice. Results are expressed as the mean±SEM. n=8-12

FIG. 6. MODY3 Knock-in mice presented mild-hyperglycemia. Glycaemia evolution of WT/WT (wild-type), WT/KI (heterozygous) and KI/KI (homozygous) from 4 to 14 weeks of age in male (A) and female (B) mice. Results are expressed as the mean±SEM. n=8-12.

FIG. 7. Fasted glycaemia was increased in MODY3 Knock-in young mice. Fasted glycaemia of WT/WT (wild-type) and KI/KI (homozygous) of 6 weeks of age in male (A) and female (B) mice. Results are expressed as the mean±SEM. n=4-16. **p<0.01, ***p<0.001 vs WT/WT.

FIG. 8. Fasted glycaemia was increased in MODY3 Knock-in adult mice. Fasted glycaemia of WT/WT (wild-type) and KI/KI (homozygous) of 12-13 weeks of age in male (A) and female (B) WT/WT and KI/KI mice. Results are expressed as the mean±SEM. n=10-16. **p<0.01, ***p<0.001 vs WT/WT.

FIG. 9. MODY3 young mice presented impaired glucose tolerance. Glucose tolerance test was performed after an intraperitoneal injection of glucose (2 g of glucose/kg body weight) at 6 weeks of age in male (A) and female (B) WT/WT (wild-type) and KI/KI (homozygous) mice. Results are expressed as the mean±SEM. n=4-16. *p<0.05, **p<0.01, ***p<0.001 vs WT/WT.

FIG. 10. MODY3 adult mice exhibit impaired glucose tolerance. Glucose tolerance test was performed after an intraperitoneal injection of glucose (2 g of glucose/kg body weight) at 12-13 weeks of age in WT/WT (wild-type) and KI/KI (homozygous) male (A) and female (B) mice. Results are expressed as the mean±SEM. n=6-12. **p<0.01, ***p<0.001 vs WT/WT.

FIG. 11. MODY3 Knock-in mice presented a reduction of fed serum insulin. Fed serum insulin levels at 14-16 weeks of age in WT/WT (wild-type) and KI/KI (homozygous) male (A) and female (B) mice. Results are expressed as the mean±SEM. n=7-12.

FIG. 12. Reduction of islet size and beta cell mass in adult MODY3 mice. Immunohistochemical detection of insulin in pancreas of 14-16-weeks-old WT/WT (wild-type) and KI/KI (homozygous) male mice. Quantification of (A) islet number, (B) mean islet area (μm²), (C) fold change β-cell mass vs wild type group. Results are expressed as the mean±SEM. n=3-4.

FIG. 13. Downregulation of HNF1α target genes expression in adult MODY3 mice. Gene expression in islets from 14-16-week-old WT/WT (wild-type) and KI/KI (homozygous) male mice. Relative expression of Hnf1α target genes: L-pk (L-pyruvate kinase), Glut2 (Glucose transporter 2) Nbat (neuroblastoma associated transcript 1), Igf1 (Insulin Like Growth Factor 1), Ins1 (insulin 1), Hnf4a (hepatocyte nuclear factor 4 alpha), Hnf1b (hepatocyte nuclear factor 1 beta), Pdx1 (pancreatic and duodenal homeobox 1), and Hnf3b (hepatocyte nuclear factor 3 beta), in (A) male and (B) female mice. Results are expressed as the mean±SEM. n=6-8. *p<0.05, **p<0.01, ***p<0.001 vs WT/WT.

FIG. 14. Intraductal administration of AAV8 vectors encoding GFP. Nine weeks-old wild type male mice were intraductally administered with 1×10\hat{ }12 vg/animal of AAV8-RIPI-GFP, AAV8-RIPII-GFP, AAV8-hlNS1.9-GFP or AAV8-hlns385-GFP vectors. Gene expression in islets from 11-week-old wild-type mice. Relative expression of GFP in islets and liver. Results are expressed as the mean±SEM. n=6-7. *p<0.005, ***p<0.001 vs AAV8-RIPI-GFP. $ p<0.05, $$ p<0.01 vs AAV8-RIPII-GFP. & p<0.05, && p<0.01 vs AAV8-hlNS1.9-GFP.

FIG. 15. Intraductal administration of AAV8 vectors encoding mmHNF1A_a under the control of rat insulin promoters. Nine weeks-old wild type male mice were intraductally administered with 1×10\hat{ }12 vg/animal of AAV8-RIPI-mmHNF1a_a or AAV8-RIPII-mmHNF1a_a vectors. Wild-type mice intraductally administered with PBS served as controls. Gene expression in islets from 17-week-old wild-type mice. Relative expression of (A) endogenous and AAV-derived Hnf1a (Hepatocyte Nuclear Factor 1-Alpha) gene, or (B) endogenous Hnf1a gene. Results are expressed as the mean±SEM. n=6-7. ***p<0.001 vs PBS.

FIG. 16. Evaluation of islet number and beta-cell mass in mice treated with AAV8-RIPI-mmHNF1a_a or AAV8-RIPII-mmHNF1a_a vectors. Nine weeks-old wild type male mice were intraductally administered with 1×10 vg/animal of AAV8-RIPI-mmHNF1a_a or AAV8-RIPII-mmHNF1a_a vectors. Wild-type mice intraductally administered with PBS served as controls. Immunohistochemical detection of insulin in pancreas of 17-weeks-old mice. Quantification of (A) islet number, (B) percentage of β-cell area relative to pancreas area. Results are expressed as the mean±SEM. n=3. ***p<0.001 vs PBS.

FIG. 17. Intraductal administration of AAV8 vectors encoding mmHNF1A_a under the control of human insulin promoters. Nine weeks-old wild type male mice were intraductally administered with 1×10\hat{ }12 vg/animal of AAV8-hlNS1.9-mmHNF1a_a or AAV8-hlns385-mmHNF1a_a vectors. Wild-type mice intraductally administered with PBS served as controls. Gene expression in islets from 13-week-old wild-type mice. Relative expression of (A) all endogenous and AAV-derived Hnf1a (Hepatocyte Nuclear Factor 1-Alpha) gene, or (B) only endogenous Hnf1a gene. Results are expressed as the mean±SEM. n=6-7. ***p<0.001 vs PBS.

FIG. 18. Evaluation of islet number and beta-cell mass in mice treated with AAV8-hlNS1.9-mmHNF1a_a or AAV8-hins385-mmHNF1a_a vectors. Nine weeks-old wild type male mice were intraductally administered with 1×10 vg/animal of AAV8-hlNS1.9-mmHNF1a_a or AAV8-hlns385-mmHNF1a_a vectors. Wild-type mice intraductally administered with PBS served as controls. Immunohistochemical detection of insulin in pancreas of 13-weeks-old mice. Quantification of (A) islet number, (B) β-cell mass. Results are expressed as the mean±SEM. n=3. **p<0.01 vs PBS.

FIG. 19. AAV-mediated counteraction of hyperglycemia in MODY3 mice. Eight weeks-old KI/KI (homozygous) mice were intraductally administered with 5×10 vg/animal of AAV8-hlns385-mmHNF1a_a vectors. WT/WT (wild-type) and KI/KI (homozygous) mice intraductally administered with PBS served as controls. Glycaemia evolution of WT/WT, KI/KI and KI/KI treated with AAV8-hlns385-mmHNFla_a from 8 to 16 weeks-old in male mice. Results are expressed as the mean±SEM. n=3-10. *p<0.05, ***p<0.001 vs WT/WT. $$ p<0.01, $$$ p<0.001 vs KI/KI treated with PBS.

FIG. 20. AAV-mediated improvement of glucose tolerance in MODY3 mice. Glucose tolerance test was performed after an intraperitoneal injection of glucose (1 g of glucose/kg body weight) at 18 weeks of age in male WT/WT (wild-type), KI/KI (homozygous) and KI/KI treated with AAV8-hlns385-mmHNFla_a mice. Results are expressed as the mean±SEM. n=3-10. *p<0.05, **p<0.01 vs WT/WT. $ p<0.05 vs KI/KI treated with PBS.

FIG. 21. Body weight evolution in MODY3 KI mice treated with AAV8-hIns385-mmHNF1a_a vectors. Eight weeks-old KI/KI (homozygous) mice were intraductally administered with 5×10 vg/animal of AAV8-hlns385-mmHNF1a_a vectors. WT/WT (wild-type) and KI/KI (homozygous) mice intraductally administered with PBS served as controls. Body weight evolution of WT/WT, KI/KI and KI/KI treated with AAV8-hlns385-mmHNF1a_a from 8 to 16 weeks-old in male mice. Results are expressed as the mean±SEM. n=3-10.

FIG. 22. MODY3 male adult mice exhibit impaired insulin secretion in vitro. In vitro insulin secretion was evaluated in isolated islets from WT/WT (wild-type) and KI/KI (homozygous) male mice (14-16 weeks of age) incubated with low and high glucose concentrations. Insulin levels were evaluated in medium (A) and isolated islets (B). Results are expressed as the mean±SEM. n=4-6. *p<0.05, **p<0.01, ***p<0.001 vs WT/WT.

FIG. 23. MODY3 male adult mice exhibit impaired insulin secretion in vivo. An insulin release test was performed after an intraperitoneal injection of glucose (3 g of glucose/kg body weight) at 15 weeks of age in WT/WT (wild-type) and KI/KI (homozygous) male mice. Results are expressed as the mean±SEM. n=4-6. *p<0.05 vs WT/WT.

FIG. 24. Increased HNF1A expression levels in islets of MODY3 mice treated with AAV8-hlNS385-mmHNF1α_a vectors. Expression levels of Hnf1a (Hepatocyte Nuclear Factor 1-Alpha) were evaluated in islets from 14-16-week-old WT/WT (wild-type), KI/KI (homozygous) and KI/KI mice treated with AAV8-hlNS385-mmHNFla_a vectors by qPCR. Results are expressed as the mean±SEM. n=6-7. ** p<0.01 vs WT/WT, $$ p<0.01 vs KI/KI treated with PBS.

FIG. 25. Normalization of HNF1A production in islets of MODY3 mice treated with AAV8-hlNS385-mmHNF1α_a vectors. HNF1a protein content was evaluated by Western-blot in islets from 14-16-week-old WT/WT (wild-type), KI/KI (homozygous) and KI/KI mice treated with AAV8-hlNS385-mmHNF1α_a vectors. (A) A representative immunoblot of HNF1a protein and the normalizer tubulin protein is shown. (B) The histograms depict the densiometric analysis of different immunoblots. Results are expressed as the mean±SEM. n=4. **p<0.01 vs WT/WT. &&& p<0.001 vs KI/KI treated with AAV8-hlNS385-mmHNF1α.

FIG. 26. AAV treatment increases HNF1a target genes expression. Expression levels of the Hnfla target genes S/c2a2 (encoding for glucose transporter 2, GLUT2), L-pk (L-pyruvate kinase) and Hnf4a (hepatocyte nuclear factor 4 alpha) in islets from 14-16-week-old male WT/WT (wild-type), KI/KI (homozygous) and KI/KI mice treated with AAV8-hlNS385-mmHNF1α_a vectors. Results are expressed as the mean±SEM. n=5-7.

FIG. 27. Amelioration of fasted glycaemia in MODY3 KI mice treated with AAV vectors. Fasted glycaemia of male WT/WT (wild-type), KI/KI (homozygous) and KI/KI mice treated with AAV8-hlNS385-mmHNF1a vectors at 15 weeks of age. Results are expressed as the mean±SEM. n=15-30. p<0.001 vs WT/WT, $$$ p<0.001 vs KI/KI treated with PBS.

FIG. 28. Counteraction of hyperglycemia in MODY3 mice treated with a low dose of AAV vectors. Eight-week-old male KI/KI (homozygous) mice were intraductally administered with 10 vg/animal of AAV8-hlNS385-mmHNF1α_a vectors. WT/WT (wild-type) and KI/KI (homozygous) mice intraductally administered with PBS served as controls. Glycaemia evolution was monitored for 6 weeks. Results are expressed as the mean±SEM. n=16-52. **p<0.01, ***p<0.001 vs WT/WT. &&p<0.01, &&&p<0.001 vs KI/KI treated with AAV8-hlNS385-mmHNF1α_a.

### EXAMPLES

### General Procedures to the Examples

### Generation of MODY3 Mice

MODY3 mice were generated using CRISPR/Cas9 technology. The gRNA, donor DNA, and Cas9 mRNA were pronuclearly microinjected in one-cell mice embryos. After Cas9-mediated double strand break and homologous recombination with the donor DNA, the two copies of miRT375 were introduced between the exon 10 and the 3′UTR of the mouse HNF1A gene.

### Mice Genotyping

Forward (GGACTTGGCCAACAGCTAGT, SEQ ID NO: 54) and reverse (GGAGGAGCAGCAGTGTCAAT; SEQ ID NO: 55) primers targeting exon 10 and the 3′ UTR of the HNF1A gene were used for genotyping of offspring. PCR reaction generated a 392 bp amplicon that was subsequently digested with EcoRV restriction enzyme. EcoRV digestion generated fragments of 257 and 80 bp in the WT allele; and of 202, 110 and 80 bp bp in the allele comprising the two miRT375 copies.

### Subject Characteristics

Male C57Bl/6J mice and MODY3 mice were used. Mice were fed ad libitum with a standard diet (2018S Teklad Global Diets@, Harlan Labs., Inc., Madison, WI, US) and kept under a light-dark cycle of 12 h (lights on at 8:00 a.m.) and stable temperature (22° C.±2). Mice were weighted weekly after weaning. Blood glucose levels were measured with a Glucometer Elite™ analyzer (Bayer, Leverkusen, Germany). For tissue sampling, mice were anesthetized by means of inhalational anesthetic isoflurane (IsoFlo®, Abbott Laboratories, Abbott Park, IL, US) and decapitated. Tissues of interest were excised and kept at −80° C. or with formalin until analysis. All experimental procedures were approved by the Ethics Committee for Animal and Human Experimentation of the Universitat Autdnoma de Barcelona.

### Recombinant AAV Vectors

Single-stranded AAV vectors of serotype 8 were produced by triple transfection of HEK293 cells according to standard methods (Ayuso, E. et al., 2010. Curr Gene Ther. 10(6):423-36). Cells were cultured in 10 roller bottles (850 cm2, flat; Corning™, Sigma-Aldrich Co., Saint Louis, MO, US) in DMEM 10% FBS to 80% confluence and co-transfected by calcium phosphate method with a plasmid carrying the expression cassette flanked by the AAV2 ITRs, a helper plasmid carrying the AAV2 rep gene and the AAV of serotypes 8 cap gene, and a plasmid carrying the adenovirus helper functions. Transgenes used were: GFP or mouse HNF1A isoform A coding-sequence driven by 1) the rat insulin promoter 1 (RIPI); 2) the rat insulin promoter 2 (RIPII); 3) the human full length insulin promoter (hlNS1.9); or 4) a shortened version of the human insulin promoter (hlNS385). AAV were purified with an optimized method based on a polyethylene glycol precipitation step and two consecutive cesium chloride (CsCl) gradients. This second-generation CsCl-based protocol reduced empty AAV capsids and DNA and protein impurities dramatically (Ayuso, E. et al., 2010. Curr Gene Ther. 10(6):423-36). Purified AAV vectors were dialyzed against PBS, filtered and stored at −80° C. Titers of viral genomes were determined by quantitative PCR following the protocol described for the AAV2 reference standard material using linearized plasmid DNA as standard curve (Lock M, et al., Hum. Gene Ther. 2010; 21:1273-1285). The vectors were constructed according to molecular biology techniques well known in the art.

### Retrograde Administration Through Pancreatic Biliary Duct

The retrograde injection via pancreatic biliary duct was conducted as previously described (Jimenez et al., Diabetologia. 2011 May; 54(5):1075-86). The animals were anesthetized by an intraperitonial injection of ketamine (100 mg/kg) and xylacine (10 mg/kg). Once the zone shaved and an incision of 2-3 cm done, the abdomen was opening through an incision through the alba line, putting an abdominal separator. The bile duct was identified. Liver lobes were separated and the bile duct was clamped in the bifurcation of the hepatic tryad to prevent the spread of viral vector to the liver. Later, a 30 G needle was introduced through the Vater papilla and retrogrally followed through biliary duct. The needle was fixed clamping the duct at the point of the intestine to secure its position and prevent the escape of viral vectors in the intestine. Slowly, the solution was injected with the corresponding dose of viral vectors. One min after injection, the clip which fixed the needle was pulled from and a drop of surgical veterinary adhesive Histoacryl (Braun, TS1050044FP) was applied at the entry point of the needle. Approximately 2 min later the clip of the biliar duct was pulled from and the abdominal wall and skin were sutured. The mice were left to recover from anesthesia on a heating mantle to prevent heat loss.

### Immunohistochemical and Morphometric Analysis

Tissues were fixed for 24 h in formalin (Panreac Qulmica), embedded in paraffin, and sectioned. Pancreas sections were incubated overnight at 4° C. with guinea pig anti-insulin (1:100; 1-8510; Sigma-Aldrich). Rabbit anti-guinea pig coupled to peroxidase (1:300; P0141; Dako) was used as secondary antibodies. The ABC peroxidase kit (Pierce) was used for immunodetection, and sections were counterstained in Mayer's hematoxylin. Images were taken at 2× for pancreatic area and 10 or 20× for islets with the Nikon Eclipse E800 microscope (Nikon, Tokyo, Japan) connected to a videocamera with a monitor with an image analysis software (analySIS 3.0; Soft Imaging System, Center Valley, PA, EEUU) and each area was quantified in μm2. The percentage of β-cell area in the pancreas was analysed in two insulin-stained sections 200 μm apart, by dividing the area of all insulin+ cells in one section by the total pancreas area of that section. β-cell mass was calculated by multiplying pancreas weight by percentage of β-cell area, as previously described (Jimenez et al, Diabetologia. 2011 May; 54(5):1075-86).

### Isolation of Pancreatic Islets

The pancreatic islets were extracted by pancreas digestion and subsequent isolation of pancreatic islets. In order to digest the pancreas, mice were sacrificed, the abdominal cavity was exposed and 3 ml of a solution of Liberase (Roche, 0104 mg/ml medium without serum M199 (Gibco-Life Technologies 10012-037)) was perfused to the pancreas via the common biliar duct. During perfusion, circulation through the Vatter ampoule was blocked by placing a clamp. Once perfused, the pancreas was isolated from the animal and kept once before being digested at 37° C. for 19 min. To stop digestion and dilute the Liberase solution, 35 ml of cold medium M199 with 10% serum (Biowest S0250-500) were added and the tube stirred for 30 s to completely disintegrate the tissue. Then, two washes with 30 ml and 10 ml respectively of M199 medium supplemented with serum were done. Then, the solution of disintegrated tissue was filtered (450 mm PGI 34-1800-09) and collected into a new tube. The filtrate with 20 ml of medium with serum was centrifuged (Eppendorf 5810R rotor A-4-62) at 200-230×g for 5 min at 4° C. The supernatant was discarded and after carefully removing all traces of the medium, the pellet was resuspended in 13 ml of Histopaque-1077 (Sigma 10771) and M199 medium without serum was added to a volume of 25 ml avoiding mixing the two phases. Then it was centrifuged (Eppendorf 5810R) at 1000×g for 24 min at 4° C. to obtain the pancreatic islets at the interface between the medium and the Histopaque and thus, they were collected with the pipette. Once isolated, the islets were washed twice with 40 ml of medium with serum and centrifuged at 1400 rpm, 2.5 min at room temperature. In the final wash the pellet with islets was resuspended in 15 ml of M199 medium. In this step, and to help their identification under the microscope, the islets were stained by adding a solution of 200 ml Dithizone to the medium (for 10 ml volume: 30 mg Dithizone (Fluka 43820), 9 ml absolute EtOH, 150 μl NH4OH and 850 μl H2O). After 5 min of incubation, islets were transferred to a petri dish and were hand-picked under the binocular microscope.

### In Vitro Glucose Stimulated Insulin Secretion

After islet isolation, islets were cultured O/N at 37° C. in RPMI 1640 medium (11 mM glucose), supplemented with 1% BSA, 2 mM glutamine, and penicillin/streptomycin in an atmosphere of 95% humidified air, 5% CO2, to allow recovery from islet isolation stress. Next, 120 islets of similar size isolated from mice of each experimental group were washed in KRBG30 buffer twice and then were handpicked and seeded in a 6-well plate containing KRB G30 for pre-culture during 2 hours at₃7° C. in an atmosphere of 95% humidified air, 5% CO2. Then, 150 μl of KRB G30 (low glucose) or KRB G300 (high glucose) were loaded in a 96-well plate (5 wells per condition). After 2 hours, 20 pre-cultured islets per well were loaded in the new 96-well plate containing low or high glucose medium and were incubated during 1 hour at 37° C. After this incubation, medium and (120 μl/well) islets were collected separately. Medium was subsequently stored at −80° C. After collection of islets, acetic acid lysis buffer was added and the mixture was frozen O/N at −80° C. For islet lysis, islets and acetic acid were boiled at 100° C. for 10 min, then spinned at 4° C. for 10 min at 12000 rpm. The supernatant was collected and stored at −80° C. Insulin content in islets and insulin concentration in culture medium were measured by ELISA.

### RNA Analysis

Total RNA was obtained from islets or liver by using Tripure isolation reagent (Roche Diagnostics Corp., Indianapolis, IN, US), and RNAeasy Microkit (Qiagen NV, Venlo, NL) for islets and RNeasy Tissue Minikit (Qiagen NV, Venlo, NL) for liver. In order to eliminate the residual viral genomes, total RNA was treated with DNAsel (Qiagen NV, Venlo, NL).

The concentration and purity of the obtained RNA was determined using a device Nanodrop (ND-1000, ThermoCientific). For RT-PCR, 1 μg of RNA samples was reverse-transcribed using Transcriptor First Strand cDNA Synthesis Kit (04379012001, Roche, California, USA). Real-time quantitative PCR was performed in a SmartCyclerII® (Cepheid, Sunnyvale, USA) using EXPRESS SYBRGreen qPCR supermix (Invitrogen™, Life Technologies Corp., Carslbad, CA, US). Data was normalized with Rplp0 values and analyzed as previously described (Pfaffl, M., Nucleic Acids Res. 2001; 29(9):e45).

### Primer Pairs

| Gene | forward primer | reverse primer |
|---|---|---|
| Rplp0 | TCCCACCTTGTCTCC | ACTGGTCTAGGACCC |
| | AG TCT | GAGAAG |
| | (SEQ ID NO: 56) | (SEQ ID NO: 57) |
| L-PK | GTTTCTTGGGCAAC | AGGAGGCAAAGATG |
| | AGGAAG | ATGTCC |
| | (SEQ ID NO: 58) | (SEQ ID NO: 59) |
| HNF4a | AGATTGACAACCTG | TGCCCATGTGTTC |
| | CTGCAG | TTGCATC |
| | (SEQ ID NO: 60) | (SEQ ID NO: 61) |
| HNF1a | TGTCACAGCACCTCA | TGTGGGCTCTTCAAT |
| | ACAAG | CAGTC |
| | (SEQ ID NO: 62) | (SEQ ID NO: 63) |
| Slc2a2 | ATC CCT TGG TTC | AAT TGC AGA CCC |
| | ATG GTT GC | AGT TGC TG |
| | (SEQ ID NO: 64) | (SEQ ID NO: 65) |

### Hormone Detection

Insulin concentrations were determined by Rat Insulin ELISA sandwich assay (90010, Crystal Chem INC. Downers Grove, IL 60515, USA).

### Glucose Tolerance Test

Awake mice were fasted overnight (16 h) and administered with an intraperitoneal injection of glucose (1 or 2 g/kg body weight). Glycemia was measured in tail vein blood samples at the indicated time points.

### In Vivo Insulin Release Test

Awake mice were fasted overnight (16 h) and administered with an intraperitoneal injection of glucose (3 g/kg body weight). Venous blood was collected from tail vein in tubes at the indicated time points and immediately centrifuged to separate serum, which was used to measure insulin levels.

### Western Blot Analysis

Islets or liver were homogenized in Lysis Buffer. Proteins were separated by 10% SDS-PAGE, and analyzed by immunoblotting with rabbit monoclonal anti-HNF1A (D7Z2Q; Cell signaling) and rabbit polyclonal anti-α-tubulin (ab4074; Abcam) antibodies. Detection was performed using ECL Plus detection reagent (Amersham Biosciences).

### Statistical Analysis

All values are expressed as mean±SEM. Differences between groups were compared by Student's t-test. Differences were considered significant at p<0.05.

### Example 1. Generation of the New MODY3 Mouse Model

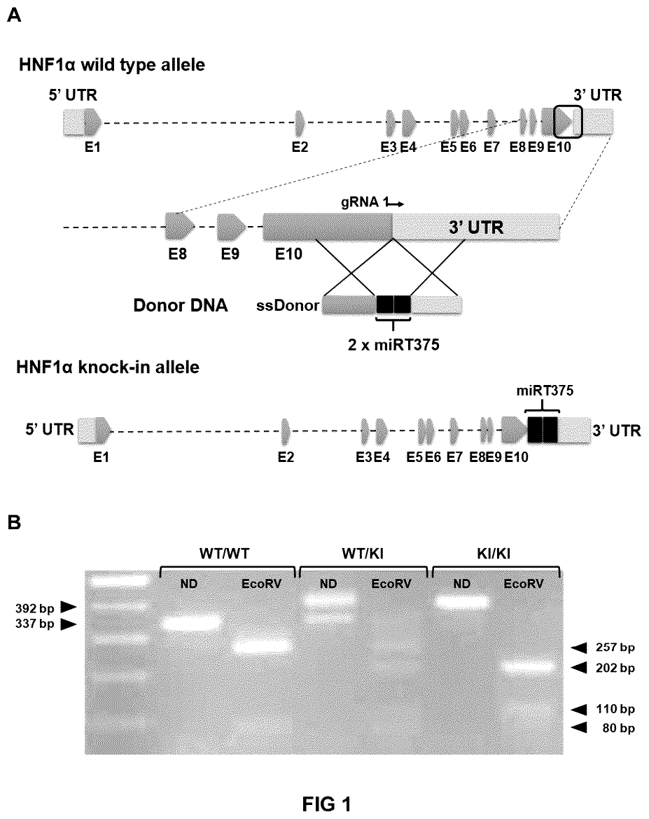

A new β-cell specific mouse model for MODY3 by means of the CRISPR/Cas9 technology was generated. To preclude production of HNF1A specifically in beta-cells, we introduced two copies of the target sequence for the beta-cell specific miRNA375 (miRT375) (SEQ ID NO: 52) upstream the 3′UTR of the HNF1A gene. Specifically, a single guided RNA (sgRNA) (SEQ ID NO: 53) was designed to target the region adjacent to exon 10 and the 3′UTR of the HNF1a gene to introduce two copies of microRNA 375 target sequence (miRT375), contained in DNA donor, by homology directed repair (HDR) (FIG. 1). miRNA are small non-coding RNAs that bind specifically to certain mRNAs preventing their translation. Incorporation of target sequences of tissue-specific miRNAs in expression cassettes has been widely used in gene therapy approaches to de-target transgene expression from undesired tissues (Jimenez, V. et al. (2018) EMBO Mol Med 10(8):8791) but to the best of our knowledge nobody has used this approach to generate disease animal models.

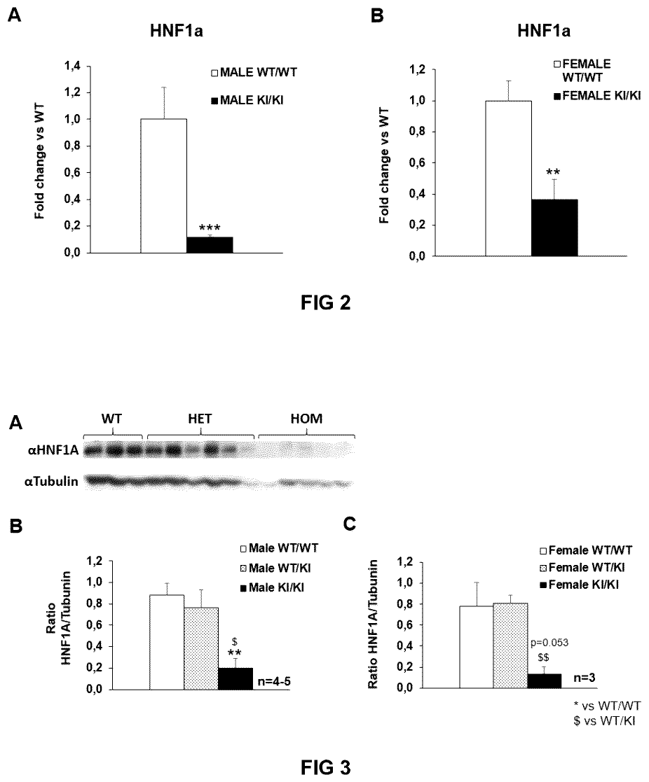

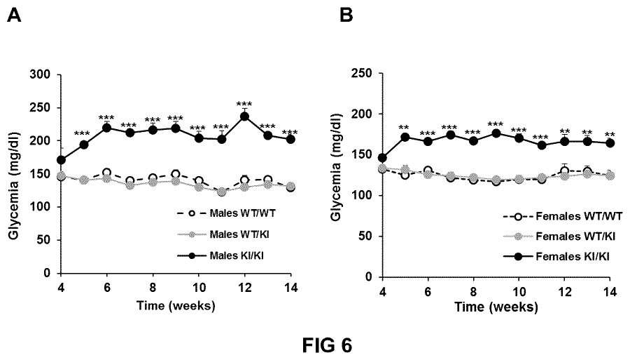

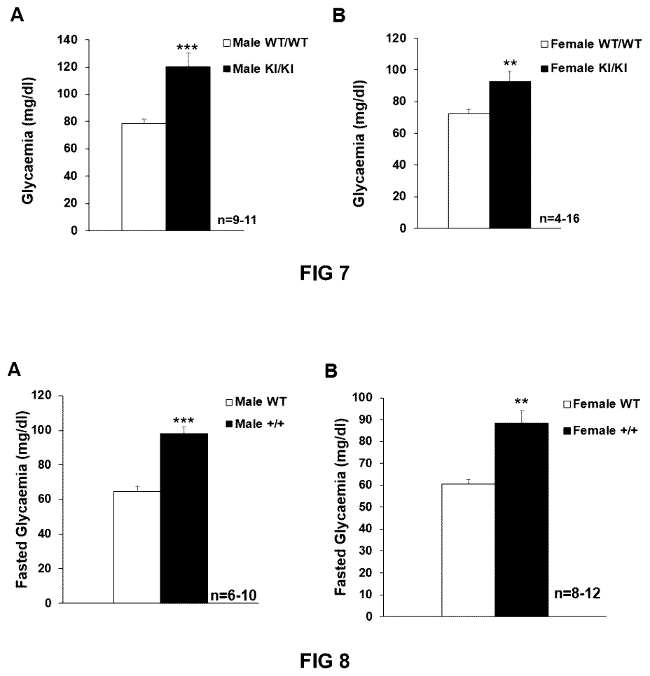

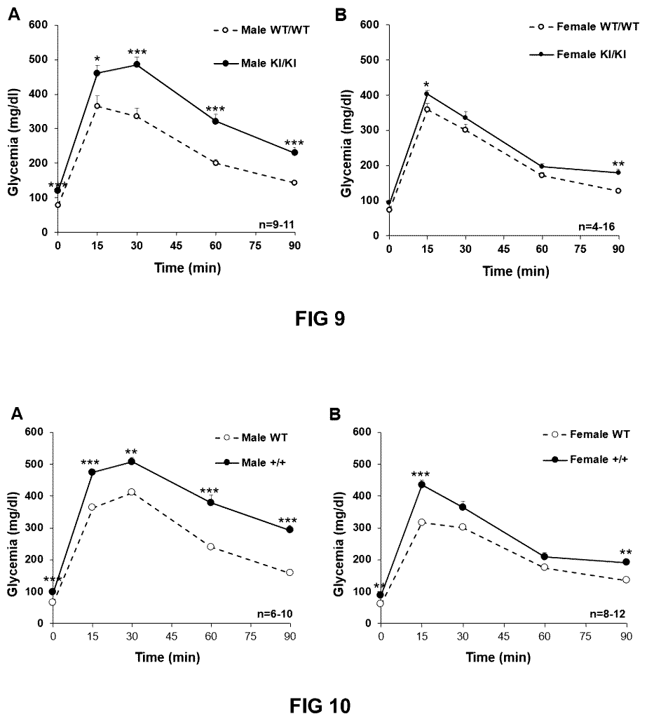

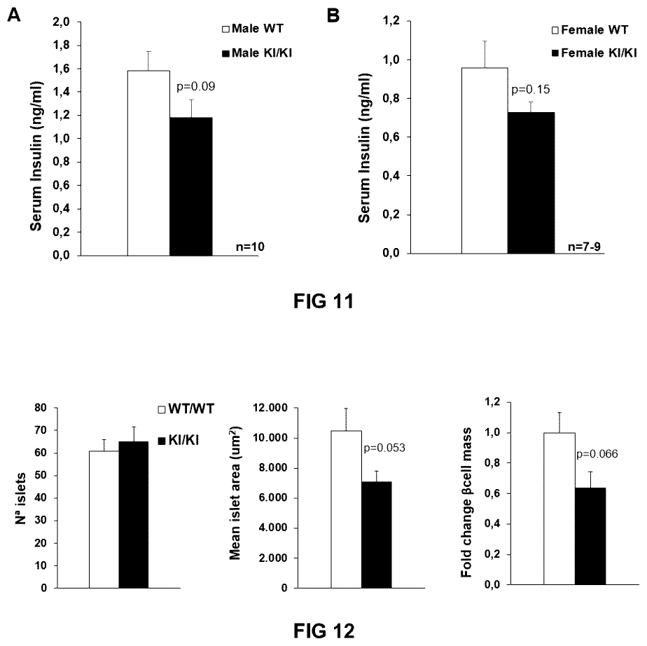

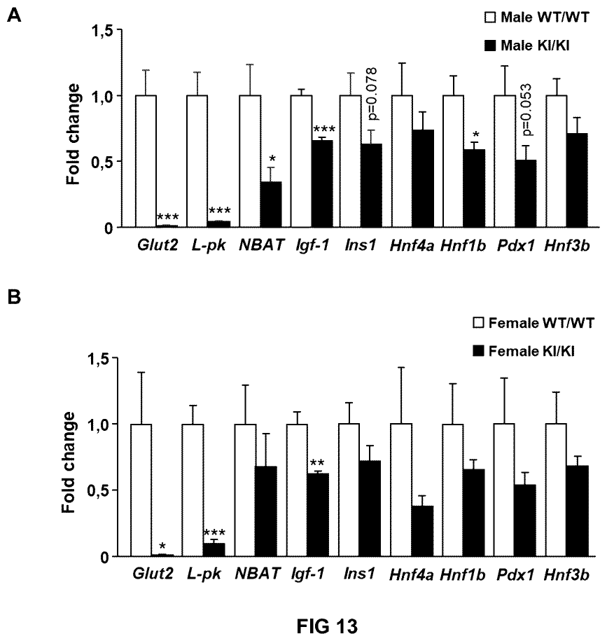

The specific gRNA, the donor DNA, and the Cas9 mRNA were pronuclearly microinjected into one-cell embryos that were subsequently transferred into recipient female mice. F0 generation was genotyped by PCR analysis using specific primers located in the flanking sequences of the knock-in site. Next, the PCR products were digested with EcoRV, leading to different patterns depending on the mice genotype (FIG. 1B). Knock in (KI) mice were backcrossed with control (C57BL6) mice in order to segregate possible CRISPR/Cas9 off-target mutations. Heterozygous mice from the F1 generation were mated again with new control (C57BL6) mice to further segregate off-targets and obtain the F2 generation. F2 heterozygous mice were mated between each other to generate the F3 in which phenotyping of the model was performed. The most important results were:
Specific downregulation of HNF1a expression and production in islets (FIGS. 2, 3 and 4)
Maintenance of body weight (FIG. 5)
Sustained mild hyperglycemia (FIG. 6)
Increased fasted glycaemia in young and adults (FIGS. 7 and 8)
Reduced glucose tolerance both in young and adults (FIGS. 9 and 10)
Reduced insulinemia (FIG. 11)
Reduced islet size and beta cell mass (FIG. 12)
Downregulation of HNF1ca target genes expression in islets (FIG. 13)

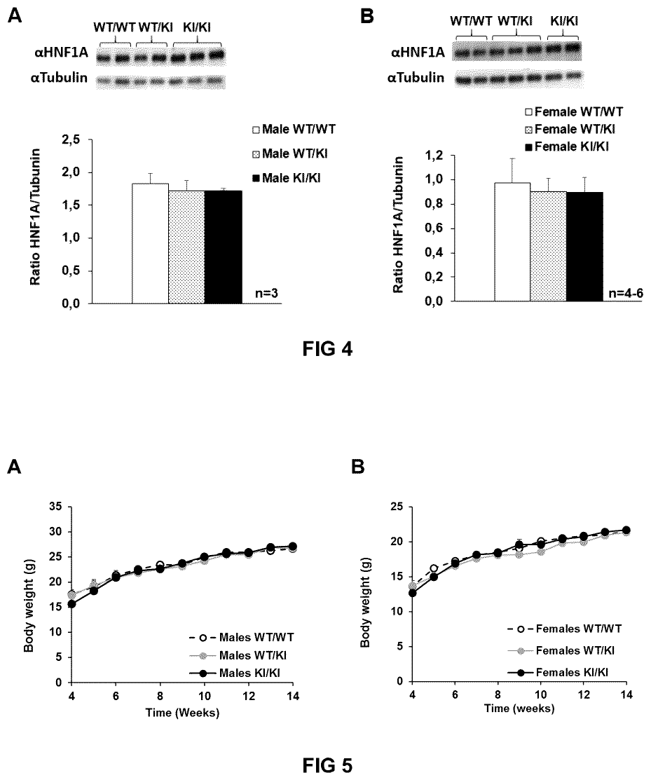

Example 2. Downregulation of HNF1A Expression and Production Levels in Islets from MODY3 Mice HNF1A expression and protein levels were analyzed in islet samples from 14 to 16-week-old MODY3 mice. Homozygous MODY3 male and female mice showed markedly reduced HNF1A expression levels and HNF1A protein content in islets (=80% reduced HNF1A protein production) (FIGS. 2 and 3). No changes in HNF1A protein content were observed in the liver of MODY3 male and female mice (FIG. 4).

### Example 3. MODY3 Mice Exhibited Mild-Hyperglycemia and Impaired Alucose Tolerance

Body weight follow-up demonstrated that wild-type, heterozygous and homozygous MODY3 mice showed similar body weight (FIG. 5). Monitoring of blood glucose levels revealed that, similarly to patients, both male and female homozygous MODY3 mice were mildly hyperglycemic under fed and fasted conditions (FIGS. 6-8).

Moreover, male and female MODY3 mice showed impaired glucose tolerance in comparison with WT mice at young and adult ages (FIGS. 9-10). Diabetic phenotype was more exacerbated in male than female MODY3 mice.

### Example 4. MODY3 Mice Showed Decreased Beta-Cell Mass and Insulinemia

To further evaluate pancreas phenotype in MODY3 mice, pancreatic sections were immunostained against insulin and morphometric analyses were performed. No striking differences in islet morphology and number of islets were detected between MODY3 and WT mice (FIG. 12A). Nevertheless, MODY3 mice showed reduced mean islet area (FIG. 12B) and β-cell mass in comparison to WT mice (FIG. 12C). In agreement, both male and female homozygous MODY3 mice showed reduced insulinemia (FIG. 11). Thus, pancreas phenotype of homozygous MODY3 mice resembles that of MODY3 patients, with defects in β-cell and insulopenia (Sanchez Malo, M. J. et al. (2019) Endocrinol Diabetes Nutr; 66(4):271-272.).

### Example 5. MODY3 Mice Showed Downregulation of HNF1A Target-Qenes and β-Cell Transcriptional Regulatory Network

In pancreatic β-cells, HNF1A has been reported to regulate expression of insulin and β-cell transcription factors as well as expression of proteins involved in glucose transport and metabolism and mitochondrial function, all of which are involved in insulin secretion (Fajans, S. S. et al. (2001). N. Engl. J. Med., 345, 971-80). Both male and female MODY3 mice showed markedly reduced expression of all HNF1A gene targets examined (FIG. 13).

Altogether, a new β-cell specific MODY3 mouse model that faithfully mimics the clinical phenotype of MODY3 patients has been developed. This new mouse model represents a useful tool to assess novel treatment strategies for MODY3.

### Example 6. Selection of Beta-Cell Specific Promoter to Drive Expression of HNF1A

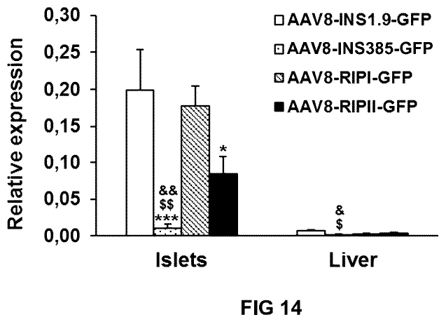

The MODY3 mouse model developed in Example 1 was used to design a suitable gene therapy approach. First, to select the most appropriate beta-cell specific promoter, AAV8 vectors encoding GFP under the control of four candidate promoters were generated. The selected promoters were the rat insulin promoter 1 (RIPI, SEQ ID NO: 16), rat insulin promoter 2 (RIPII, SEQ ID NO: 17), the full-lenght human insulin promoter (hlNS1.9, SEQ ID NO: 18), and a 385 bp fragment of the human insulin promoter (hlns385, SEQ ID NO: 20). Expression cassettes encoding GFP under the control of either RIPI, RIPII, hlNS1.9 or hlns385 promoters and flanked by the inverted terminal repeats (ITRs) of AAV2 were generated. AAV8-GFP vectors (AAV8-RIPI-GFP, AAV8-RIPII-GFP, AAV8-hlNS1.9-GFP and AAV8-hlns385-GFP) were produced by triple transfection in HEK293 cells. To evaluate the strength of the promoters and beta-cell specificity of the RIPI, RIPII, hlNS1.9 and hlns385 promoters, wild type mice were administered intraductally with AAV8-RIPI-GFP, AAV8-RIPII-GFP, AAV8-hlNS1.9-GFP or AAV8-hlns385-GFP vectors. Although all vectors promoted specific GFP overexpression in islets (FIG. 14), RIPI, RIPII and hlNS1.9 mediated higher GFP expression levels in islets than the hIns385 promoter.

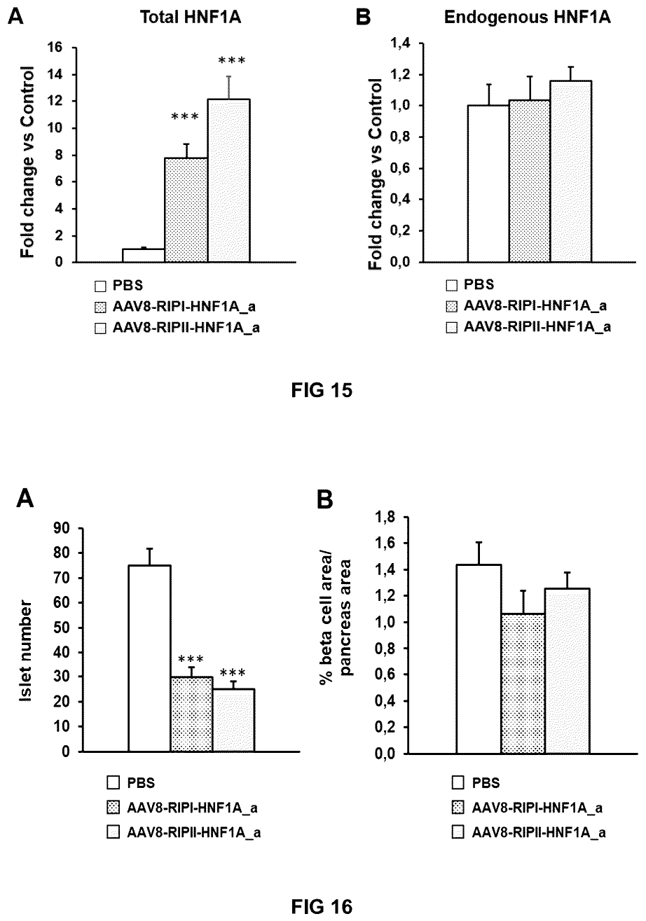

First, expression cassettes encoding the Mus musculus hepatocyte nuclear factor 1A isoform A (HNF1A_a) under the control of either RIPI or RIPII promoters and flanked by the inverted terminal repeats (ITRs) of AAV2 were generated (SEQ ID NO: 47 and 48). AAV8 vectors (AAV8-RIPI-HNF1A_a and AAV8-RIPII-HNF1A_a) were produced by triple transfection in HEK293 cells. To evaluate whether RIPI and RIPII were able to mediate HNF1A_a expression in beta-cells and to assess if this overexpression was safe, wild type mice were administered intraductally with AAV8-RIPI-HNF1A_a or AAV8-RIPII-HNF1A_a vectors. A control group administered intraductally with PBS served as control. Although both vectors promoted specific HNF1A overexpression in islets (FIG. 15), animals treated with AAV8-RIPI-HNF1A_a or AAV8-RIPII-HNF1A_avectors showed reduced islet number and beta cell mass in comparison with control mice (FIG. 16).

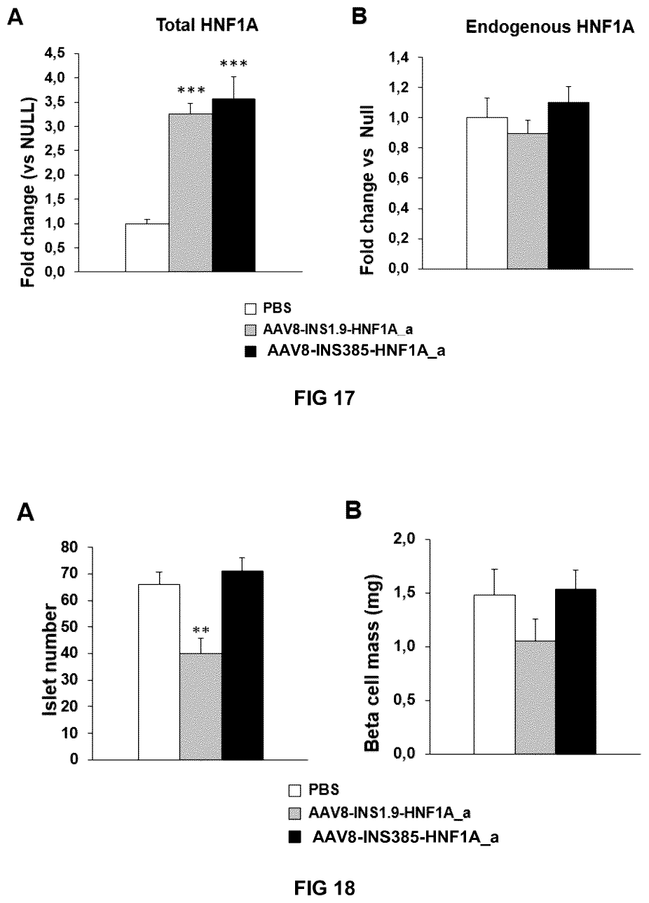

Next, expression cassettes encoding the Mus musculus hepatocyte nuclear factor 1A isoform A (HNF1A_a) under the control of either hlNS1.9 or hlns385 promoters and flanked by the inverted terminal repeats (ITRs) of AAV2 were generated (SEQ ID NO: 49 and 50). AAV8 vectors (AAV8-hlNS1.9-HNF1A_a and AAV8-hlns385-HNF1A_a) were produced by triple transfection in HEK293 cells. Wild type mice were administered intraductally with AAV8-hlNS1.9-HNF1A_a or AAV8-hlns385-HNF1A_a vectors. A control group administered intraductally with PBS served as control. Mice treated intraductally with AAV8-hlNS1.9-HNF1A_a or AAV8-hlns385-HNF1A_a vectors showed increased expression levels of HNF1A in islets (FIG. 17). However, mice treated intraductally with AAV8-hlNS1.9-HNF1A_a vectors showed decreased number of islets and β-cell mass (FIG. 18). These observations further confirmed the results obtained in WT mice treated intraductally with AAV8-RIPI-HNF1A_a or AAV8-RIPII-HNF1A_a and highlight that high overexpression of HNF1A may cause deleterious effects in β-cells. Therefore, AAV8-hlns385-HNF1A_a were chosen to evaluate the therapeutic efficacy of gene therapy for MODY3.

### Example 7. Reversal of MODY3

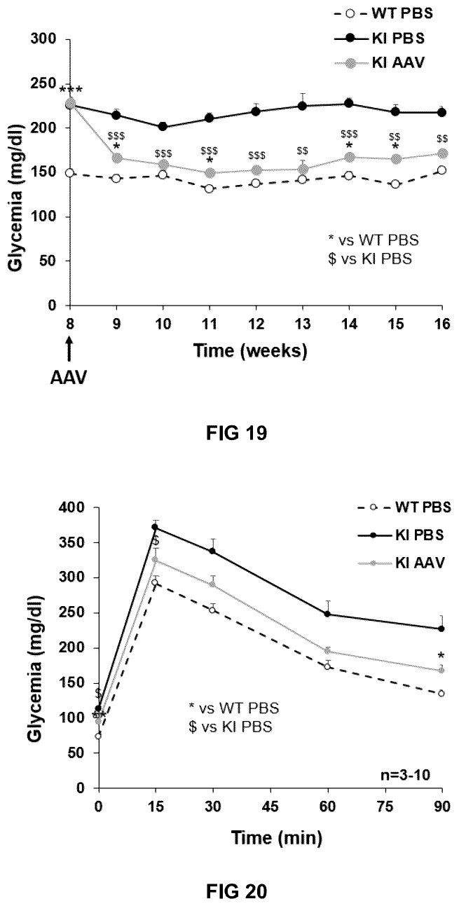

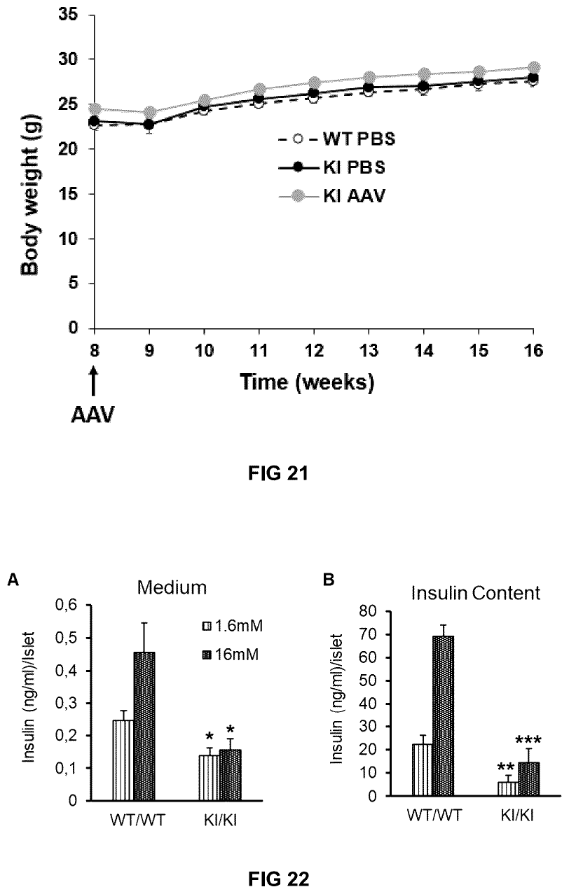

Antidiabetic therapeutic efficacy of AAV8-hlns385-HNF1A_a vectors was evaluated in the MODY3 KI mouse model. Wild type (WT) mice were used as healthy controls, and homozygous KI mice administered with PBS served as MODY3 disease controls. Noticeably, KI MODY3 mice treated with AAV8-hlns385-HNF1A_a vectors showed counteraction of the mild hyperglycemia characteristic of the disease model (FIG. 19). Moreover, MODY3 mice treated with the therapeutic vector also showed improvement of glucose tolerance (FIG. 20). No changes in body weight were observed among experimental groups (FIG. 21).

### Example 8. MODY3 Mice Exhibited Reduced Islet Insulin Content and Impaired Insulin Secretion

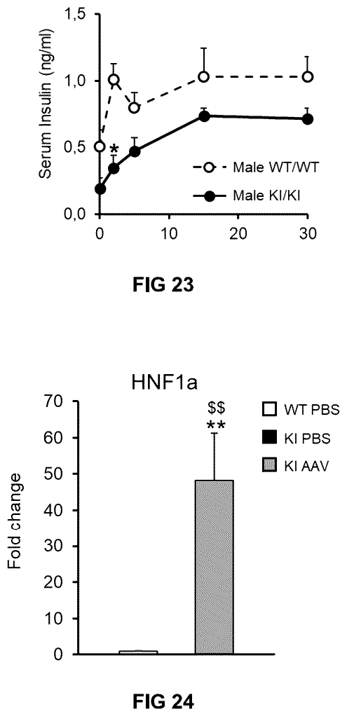

To further phenotype MODY3 KI mice, insulin secretion was evaluated both in vitro and in vivo. To this end, islets from male wild-type and MODY3 mice were incubated with low (1.6 mM) or high glucose (16 mM) and insulin content in islets as well as in the culture medium was analyzed. Islets from MODY3 mice showed decreased insulin content and reduced secretion of insulin into the culture medium at low glucose (FIG. 22). Moreover, while high glucose markedly increased insulin content in WT islets and insulin secretion, this response was blunted in islets from MODY3 mice (FIG. 22). MODY3 mice also showed reduced insulin release in vivo (FIG. 23). In particular, the first phase of insulin secretion in response to glucose was greatly diminished in these mice (FIG. 23), suggesting an impaired secretory response by beta-cells.

### Example 9. Increased HNF1A Expression and Protein Content in Islets from MODY3 Mice Treated with AAV8-Hlns385-HNF1A a Vectors

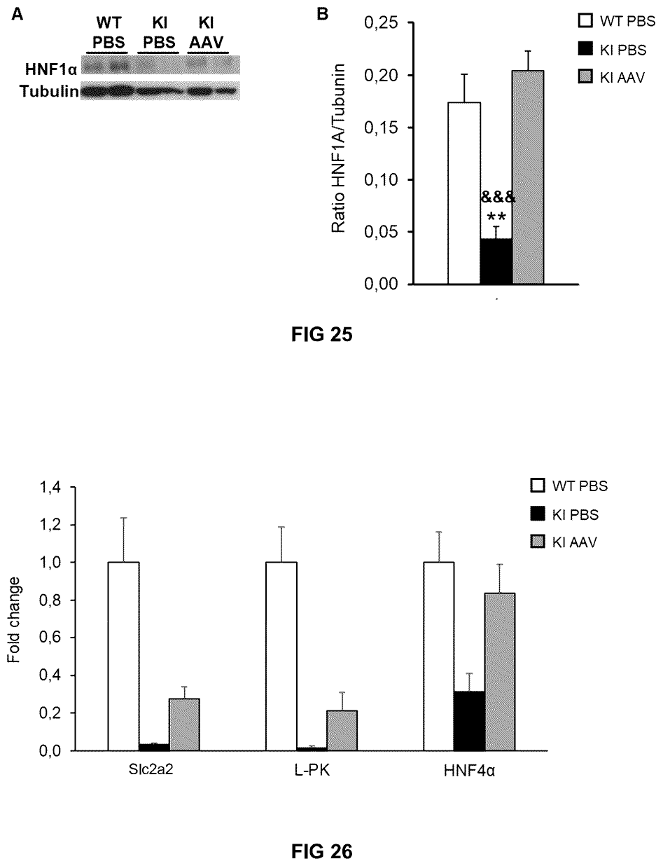

HNF1A expression levels and protein content were analyzed in islet samples from 14 to 16-week-old male wild-type, MODY3 and MODY3 mice treated with AAV8-hlNS385-mmHNF1a vectors. MODY3 mice treated with AAV8-hlns385-HNF1A_a vectors showed markedly increased HNF1A expression levels and HNF1A protein content in islets compared with MODY3 mice treated intraductally with PBS (FIGS. 24 and 25). Noticeably, HNF1A protein content in islets was normalized by the AAV treatment (FIG. 25). In addition, expression of the HNF1A gene targets S/c2a2 (encoding for glucose transporter 2, GLUT2), L-pk (L-pyruvate kinase) and Hnf4a (hepatocyte nuclear factor 4 alpha), was also increased in MODY3 mice treated with AAV8-hlns385-HNF1A_a vectors (FIG. 26).

### Example 10. MODY3 Mice Treated with AAV8-Hlns385-HNF1A Avectors Exhibited Improved Fasted Mild-Hyperglycemia

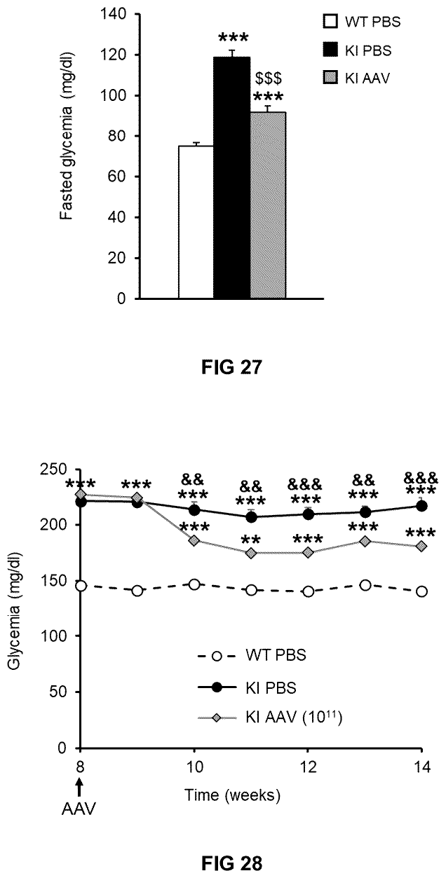

In agreement with counteraction of mild fed hyperglycemia (FIG. 19), male MODY3 mice treated with AAV8-hlns385-HNF1A_a vector also showed markedly reduced glycemia under fasted conditions (FIG. 27).

### Example 11. Reversal of MODY3 at Lower AAV Dose

Next, antidiabetic therapeutic efficacy of AAV8-hlns385-HNF1A_a vectors was evaluated in the MODY3 KI mouse model at a lower dose. Wild type (WT) mice were used as healthy controls, and homozygous KI mice administered with PBS served as MODY3 disease controls. Noticeably, MODY3 KI mice treated with AAV8-hlns385-HNF1A_a vectors at 10 vg/mouse showed counteraction of the mild hyperglycemia characteristic of the disease model (FIG. 28), similarly to treatment with higher 5×10 vg/animal dose (FIG. 19).

### Sequences

| SEQ ID NO: | Description of the sequence |
|---|---|
| 1 | Amino acid sequence of homo sapiens HNF1A isoform a |
| 2 | Amino acid sequence of homo sapiens HNF1A isoform b |
| 3 | Amino acid sequence of homo sapiens HNF1A isoform c |
| 4 | Amino acid sequence of homo sapiens HNF1A isoform 4 |
| 5 | Amino acid sequence of homo sapiens HNF1A isoform 5 |
| 6 | Amino acid sequence of homo sapiens HNF1A isoform 6 |
| 7 | Amino acid sequence of homo sapiens HNF1A isoform 7 |
| 8 | Amino acid sequence of homo sapiens HNF1A isoform 8 |
| 9 | Amino acid sequence of mus musculus HNF1A isoform H3BL72 |
| 10 | Amino acid sequence of mus musculus HNF1A isoform H3BKV2 |
| 11 | Amino acid sequence of canis lupus familiaris HNF1A |
| 12 | Nucleotide sequence of homo sapiens HNF1A |
| 13 | Nucleotide sequence of mus musculus HNF1A |
| 14 | Nucleotide sequence of canis lupus familiaris HNF1A |
| 15 | Codon optimized nucleotide sequence of homo sapiens HNF1A isoform a |
| 16 | rat insulin promoter 1 |
| 17 | rat insulin promoter 2 |
| 18 | full-lenght human insulin promoter (hINS1.9) |
| 19 | positions −385 to +24 in the human insulin promoter |
| 20 | positions −385 to −1 in the human insulin promoter |
| 21 | Nucleotide sequence encoding miRT-122a |
| 22 | Nucleotide sequence encoding miRT-152 |
| 23 | Nucleotide sequence encoding miRT-199a-5p |
| 24 | Nucleotide sequence encoding miRT-199a-3p |
| 25 | Nucleotide sequence encoding miRT-215 |
| 26 | Nucleotide sequence encoding miRT-192 |
| 27 | Nucleotide sequence encoding miRT-148a |
| 28 | Nucleotide sequence encoding miRT-194 |
| 29 | Nucleotide sequence encoding miRT-1 |
| 30 | Nucleotide sequence encoding miRT-133a |
| 31 | Nucleotide sequence encoding miRT-206 |
| 32 | Nucleotide sequence encoding miRT-208-5p |
| 33 | Nucleotide sequence encoding miRT-208a-3p |
| 34 | Nucleotide sequence encoding miRT-499-5p |
| 35 | Nucleotide sequence of CAG promoter |
| 36 | Nucleotide sequence of CMV promoter |
| 37 | Nucleotide sequence of chimeric intron composed of introns |
| | from human β-globin and immunoglobulin heavy chain genes |
| 38 | mini-CMV promoter |
| 39 | EF1α promoter |
| 40 | RSV promoter |
| 41 | SV40 polyadenylation signal |
| 42 | Rabbit β-globin polyadenylation signal |
| 43 | Nucleotide sequence of CMV enhancer |
| 44 | Truncated AAV2 5′ ITR |
| 45 | Truncated AAV2 3′ ITR |
| 46 | CMV promoter and CMV enhancer sequence |
| 47 | RIPI-HNF1A gene construct |
| 48 | RIPII- HNF1A gene construct |
| 49 | hIns1.9- HNF1A gene construct |
| 50 | hIns385- HNF1A gene construct |
| 51 | Amino acid sequence of mus musculus HNF1A |
| 52 | Target sequence of mir-375 |
| 53 | sgRNA for MODY3 model |
| 54, 55 | Forward and reverse primers targeting exon 10 and the 3′ UTR of the HNF1A gene |
| 56, 57 | Rplp0 forward and reverse primers |
| 58, 59 | L-PK forward and reverse primers |
| 60, 61 | HNF4a forward and reverse primers |
| 62, 63 | HNF1a forward and reverse primers |
| 64, 65 | Slc2a2 forward and reverse primers |

AAV2 5′ ITR (SEQ ID NO: 30)
gcgcgctcgctcgctcactgaggccgcccgggcaaagcccgggcgtcgggcgacctttggtcgcccgg
cctcagtgag cgagcgagcg cgcagagagg gagtggccaa ctccatcact aggggttcct
AAV2 3′ ITR (SEQ ID NO: 31)
aggaacccct agtgatggag ttggccactc cctctctgcg cgctcgctcg ctcactgagg
ccgggcgacc aaaggtcgcc cgacgcccgg gctttgcccg ggcggcctca gtgagcgagc
gagcgcgc
Rabbit β-globin polyadenylation signal (3′ UTR and flanking region
of rabbit beta-globin, including polyA signal)
(SEQ ID NO: 33)
gatctttttccctctgccaaaaattatggggacatcatgaagccccttgagcatctgacttctggcta
ataaaggaaatttattttcattgcaatagtgtgttggaattttttgtgtctctcactcggaaggacat
atgggagggcaaatcatttaaaacatcagaatgagtatttggtttagagtttggcaacatatgcccat
atgctggctgccatgaacaaaggttggctataaagaggtcatcagtatatgaaacagccccctgctgt
ccattccttattccatagaaaagccttgacttgaggttagattttttttatattttgttttgtgttat
ttttttctttaacatccctaaaattttccttacatgttttactagccagatttttcctcctctcctga
ctactcccagtcatagctgtccctcttctcttatggagatc
miRT sequences
miRT-122a (SEQ ID NO: 29):
5′ CAAACACCATTGTCACACTCCA 3′,
target for the microRNA-122a
(Accession Number to the miRBase database MI0000442), which is
expressed in the liver.
miRT-152 (SEQ ID NO: 30):
5′ CCAAGTTCTGTCATGCACTGA 3′,
target for the microRNA-152 (MI0000462),
which is expressed in the liver.
miRT-199a-5p (SEQ ID NO: 31):
5′ GAACAGGTAGTCTGAACACTGGG 3′,
target for the microRNA 199a
(MI0000242), which is expressed in the liver.
miRT-199a-3p (SEQ ID NO: 32):
5′ TAACCAATGTGCAGACTACTGT 3′,
target for the microRNA-199a
(MI0000242), which is expressed in the liver.
miRT-215 (SEQ ID NO: 33):
5′ GTCTGTCAATTCATAGGTCAT 3′,
target for the microRNA-215 (MI0000291),
which is expressed in the liver.
miRT-192 (SEQ ID NO: 34):
5′ GGCTGTCAATTCATAGGTCAG 3′,
target for the microRNA-192 (MI0000234),
which is expressed in the liver.
miRT-148a (SEQ ID NO: 35):
5′ ACAAAGTTCTGTAGTGCACTGA 3′,
target for the microRNA-148a
(MI0000253), which is expressed in the liver.
miRT-194 (SEQ ID NO: 36):
5′ TCCACATGGAGTTGCTGTTACA 3′,
target for the microRNA-194
(MI0000488), which is expressed in the liver.
miRT-133a (SEQ ID NO: 38):
5′ CAGCTGGTTGAAGGGGACCAAA 3′,
target for the microRNA-133a
(MI0000450), which is expressed in the heart.
miRT-206 (SEQ ID NO: 39):
5′ CCACACACTTCCTTACATTCCA 3′,
target for the microRNA-206 (MI0000490),
which is expressed in the heart.
miRT-1 (SEQ ID NO: 37):
5′ TTACATACTTCTTTACATTCCA 3′,
target for the microRNA-1 (MI0000651),
which is expressed in the heart.
miRT-208a-5p (SEQ ID NO: 40):
5′ GTATAACCCGGGCCAAAAGCTC 3′,
target for the microRNA-208a
(MI0000251), which is expressed in the heart.
miRT-208a-3p (SEQ ID NO: 41):
5′ ACAAGCTTTTTGCTCGTCTTAT 3′,
target for the microRNA-208a
(MI0000251), which is expressed in the heart.
miRT-499-5p (SEQ ID NO: 42):
5′ AAACATCACTGCAAGTCTTAA 3′,
target for the microRNA-499
(MI0003183), which is expressed in the heart.
RIPI-HNF1A gene construct (SEQ ID NO: 47)
cgccgggttttgtggaagtagagatagaggagaagggaccattacatgtcctgctgcctgagttctgc
tttccttctccctttgaaggtgagctggggtctcagctgagctaagaatccagctatcaatagaaact
atgaaacagttccagggacaaagataccaggtccccaacaactgcaactttctgggaaatgaggtgga
aagtgctcagccaaggaaaaagagggccttaccctctctgggacaatgattgtgctgtgaactgcttc
atcaggccatctggccccttgttaataatctaattaccctaggtctaagtagagttgctgacgtccaa
tgagcgctttctgcagacttagcactaggcaagtgtttggaaattacggcttcggcccctctcgccat
ctgcctacctacccctcctagagcccttaatgggccaaacggcaaagtccagggggcagagaggaggt
gctttggactataaagctagtggagacccagtaactcccaaccctaacgggatgatatcctcgaggct
agcgaattcgccaccatggtttctaagctgagccagctgcagacggagctcctggctgccctgctcga
gtctggcctgagcaaagaggccctgatccaggccttgggggagccagggccctacctgatggttggag
agggtcccctggacaagggggagtcctgcggtgggagtcgaggggacctgaccgagttgcctaatggc
cttggagaaacgcgtggctctgaagatgacacggatgacgatggggaagacttcgcgccacccattct
gaaagagctggagaacctcagcccagaggaggcagcccaccagaaagccgtggtggagtcacttcttc
aggaggacccatggcgcgtggcgaagatggtcaagtcgtacttgcagcagcacaacatcccccagcgg
gaggtggtggacaccacgggtctcaaccagtcccacctgtcacagcacctcaacaagggcacacccat
gaagacacagaagcgggccgctctgtacacctggtacgtccgcaagcagcgagaggtggctcagcaat
tcacccacgcagggcagggcggactgattgaagagcccacaggcgatgagctgccaactaagaagggg
cgtaggaaccggttcaagtggggccccgcatcccagcagatcctgttccaggcctacgagaggcaaaa
aaaccccagcaaggaagagcgagagaccttggtggaggagtgtaatagggcggagtgcatccagaggg
gggtgtcaccatcgcaggcccaggggctaggctccaaccttgtcacggaggtgcgtgtctacaactgg
tttgccaaccggcgcaaggaggaagccttccggcacaagttggccatggacacctataacggacctcc
accggggccaggcccgggccctgcgctgcctgctcacagttcccccggcctgcccacaaccaccctct
ctcccagtaaggtccacggtgtacggtacggacagtctgcaaccagtgaggcagccgaggtgccctcc
agcagcggaggtcccttagtcacagtgtctgcggccttacaccaagtatcccccacaggcctggagcc
cagcagcctgctgagcacagaggccaagctggtctcagccacggggggtcccctgcctcccgtcagca
ccctgacagcactgcacagcttggagcagacatctccgggtctcaaccagcagccgcagaaccttatc
atggcctcgctacctggggtcatgaccatcgggcccggggagcctgcctccctgggacccacgttcac
gaacacgggcgcctccaccctggttatcggtctggcctccactcaggcacagagcgtgcctgtcatca
acagcatggggagtagcctgaccacgctgcagccggtccagttttcccaaccactgcatccctcctat
cagcagcctctcatgccccccgtacagagccacgtggcccagagccccttcatggcaaccatggccca
gctgcagagcccccacgccttatacagccacaagcctgaggtggcccagtacacgcacaccagcctgc
tcccgcagaccatgttgatcacagacaccaacctcagcacccttgccagcctcacacccaccaagcag
gtcttcacctcagacacagaggcctccagtgagcccgggcttcacgagccaccctctccagccaccac
catccacatccccagccaggacccgtcgaacatccagcacctgcagcctgctcaccggctcagcacca
gtcccacagtgtcctccagcagcctggtgttgtatcagagttccgactccaacgggcacagccacctg
ctgccatccaaccatagtgtcatcgagacttttatctccacccagatggcctcctcttcccagtaagg
taccgcggccgcgttaacacgcgtgatatccacgtggagctcgctgatcagcctcgactgtgccttct
agttgccagccatctgttgtttgcccctcccccgtgccttccttgaccctggaaggtgccactcccac
tgtcctttcctaataaaatgaggaaattgcatcgcattgtctgagtaggtgtcattctattctggggg
gtggggtggggcaggacagcaagggggaggattgggaagacaatagcaggcatgctggggatgcggtg
ggctctatgg
5′ ITR: 1-128 bp
RIPI promoter: 137-665 bp
Mus musculus HNF1A_a coding sequence: 693-2579 bp
Bovine growth hormonpe polyA: 2639-2863 bp
3′ ITR: 2879-3006 pb
RIPII-HNF1A gene construct (SEQ ID NO: 48)
ggatcccccaaccactccaagtggaggctgagaaaggttttgtagctgggtagagtatgtactaagag
atggagacagctggctctgagctctgaagcaagcacctcttatggagagttgctgaccttcaggtgca
aatctaagatactacaggagaatacaccatggggcttcagcccagttgactcccgagtgggctatggg
tttgtggaaggagagatagaagagaagggacctttcttcttgaattctgctttccttctacctctgag
ggtgagctggggtctcagctgaggtgaggacacagctatcagtgggaactgtgaaacaacagttcaag
ggacaaagttactaggtcccccaacaactgcagcctcctggggaatgatgtggaaaaatgctcagcca
aggacaaagaaggcctcaccctctctgagacaatgtcccctgctgtgaactggttcatcaggccaccc
aggagcccctattaagactctaattaccctaaggctaagtagaggtgttgttgtccaatgagcacttt
ctgcagacctagcaccaggcaagtgtttggaaactgcagcttcagcccctctggccatctgctgatcc
acccttaatgggacaaacagcaaagtccaggggtcagggggggggtgctttggactataaagctagtg
gggattcagtaacccccagccctaagatatcctcgaggctagcgaattcgccaccatggtttctaagc
tgagccagctgcagacggagctcctggctgccctgctcgagtctggcctgagcaaagaggccctgatc
caggccttgggggagccagggccctacctgatggttggagagggtcccctggacaagggggagtcctg
cggtgggagtcgaggggacctgaccgagttgcctaatggccttggagaaacgcgtggctctgaagatg
acacggatgacgatggggaagacttcgcgccacccattctgaaagagctggagaacctcagcccagag
gaggcagcccaccagaaagccgtggtggagtcacttcttcaggaggacccatggcgcgtggcgaagat
ggtcaagtcgtacttgcagcagcacaacatcccccagcgggaggtggtggacaccacgggtctcaacc
agtcccacctgtcacagcacctcaacaagggcacacccatgaagacacagaaggggccgctctgtaca
cctggtacgtccgcaagcagcgagaggtggctcagcaattcacccacgcagggcagggcggactgatt
gaagagcccacaggcgatgagctgccaactaagaaggggcgtaggaaccggttcaagtggggccccgc
atcccagcagatcctgttccaggcctacgagaggcaaaaaaaccccagcaaggaagagcgagagacct
tggtggaggagtgtaatagggcggagtgcatccagaggggggtgtcaccatcgcaggcccaggggcta
ggctccaaccttgtcacggaggtgcgtgtctacaactggtttgccaaccggcgcaaggaggaagcctt
ccggcacaagttggccatggacacctataacggacctccaccggggccaggcccgggccctgcgctgc
ctgctcacagttcccccggcctgcccacaaccaccctctctcccagtaaggtccacggtgtacggtac
ggacagtctgcaaccagtgaggcagccgaggtgccctccagcagcggaggtcccttagtcacagtgtc
tgcggccttacaccaagtatcccccacaggcctggagcccagcagcctgctgagcacagaggccaagc
tggtctcagccacggggggtcccctgcctcccgtcagcaccctgacagcactgcacagcttggagcag
acatctccgggtctcaaccagcagccgcagaaccttatcatggcctcgctacctggggtcatgaccat
cgggcccggggagcctgcctccctgggacccacgttcacgaacacgggcgcctccaccctggttatcg
gtctggcctccactcaggcacagagcgtgcctgtcatcaacagcatggggagtagcctgaccacgctg
cagccggtccagttttcccaaccactgcatccctcctatcagcagcctctcatgccccccgtacagag
ccacgtggcccagagccccttcatggcaaccatggcccagctgcagagcccccacgccttatacagcc
acaagcctgaggtggcccagtacacgcacaccagcctgctcccgcagaccatgttgatcacagacacc
aacctcagcacccttgccagcctcacacccaccaagcaggtcttcacctcagacacagaggcctccag
tgagcccgggcttcacgagccaccctctccagccaccaccatccacatccccagccaggacccgtcga
acatccagcacctgcagcctgctcaccggctcagcaccagtcccacagtgtcctccagcagcctggtg
ttgtatcagagttccgactccaacgggcacagccacctgctgccatccaaccatagtgtcatcgagac
ttttatctccacccagatggcctcctcttcccagtaaggtaccgcggccgcgttaacacgcgtgatat
ccacgtggagctcgctgatcagcctcgactgtgccttctagttgccagccatctgttgtttgcccctc
ccccgtgccttccttgaccctggaaggtgccactcccactgtcctttcctaataaaatgaggaaattg
catcgcattgtctgagtaggtgtcattctattctggggggtggggggggcaggacagcaagggggagg
attgggaagacaatagcaggcatgctggggatgcggtgggctctatgg
5′ ITR: 1-128 bp
RIPII promoter: 138-844 bp
Mus musculus HNF1A_a coding sequence: 872-2758 bp
Bovine growth hormonpe polyA: 2818-3042 bp
3′ ITR: 3058-3185 bp
hIns1.9-HNF1A gene construct (SEQ ID NO: 49)
ccccggccgacaacactggcaaactcctactcatccacgaaggccctcctgggcatggtggtccttcc
cagcctggcagtctgttcctcacacaccttgttagtgcccagcccctgaggttgcagctgggggtgtc
tctgaagggctgtgagcccccaggaagccctggggaagtgcctgccttgcctccccccggccctgcca
gcgcctggctctgccctcctacctgggctccccccatccagcctccctccctacacactcctctcaag
gaggcacccatgtcctctccagctgccgggcctcagagcactgtggcgtcctggggcagccaccgcat
gtcctgctgtggcatggctcagggtggaaagggcggaagggaggggtcctgcagatagctggtgccca
ctaccaaacccgctcggggcaggagagccaaaggctgggtgtgtgcagagcggccccgagaggttccg
aggctgaggccagggtgggacatagggatgcgaggggccggggcacaggatactccaacctgcctgcc
cccatggtctcatcctcctgcttctgggacctcctgatcctgcccctggtgctaagaggcaggtaggg
gctgcaggcagcagggctcggagcccatgccccctcaccatgggtcaggctggacctccaggtgcctg
ttctggggagctgggagggccggaggggtgtaccccaggggctcagcccagatgacactatgggggtg
atggtgtcatgggacctggccaggagaggggagatgggctcccagaagaggagtgggggctgagaggg
tgcctggggggccaggacggagctgggccagtgcacagcttcccacacctgcccacccccagagtcct
gccgccacccccagatcacacggaagatgaggtccgagtggcctgctgaggacttgctgcttgtcccc
aggtccccaggtcatgccctccttctgccaccctggggagctgagggcctcagctggggctgctgtcc
taaggcaggggggaactaggcagccagcagggaggggacccctccctcactcccactctcccaccccc
accaccttggcccatccatggcggcatcttgggccatccgggactggggacaggggtcctggggacag
gggtgtggggacaggggtcctggggacaggggtctggggacaggggtcctggggacaggggtgtgggg
acaggggtgtggggacaggggtgtggggacaggggtcctggggacaggggtctggggacaggggtctg
aggacaggggtgtggggacaggggtgtggggacaggggtgtggggacaggggtgtggggacaggggtc
tggggacaggggtccgggggacaggggtgtggggacaggggtgtggggacaggggtgtggggacaggg
gtctggggacaggggtgtggggacaggggtcctggggacaggggtgtggggataggggtgtggggaca
ggggtgtggggacaggggtgtggggacaggggtctggggacagcagcgcaaagagccccgccctgcag
cctccagctctcctggtctaatgtggaaagtggcccaggtgagggctttgctctcctggagacatttg
cccccagctgtgagcagggacaggtctggccaccgggcccctggttaagactctaatgacccgctggt
cctgaggaagaggtgctgacgaccaaggagatcttcccacagacccagcaccagggaaatggtccgga
aattgcagcctcagcccccagccatctgccgacccccccaccccaggccctaatgggccaggcggcag
gggttgagaggtaggggagatgggctctgagactataaagccagcgggggcccagcagccctcgatat
cctcgaggctagcgaattcgccaccatggtttctaagctgagccagctgcagacggagctcctggctg
ccctgctcgagtctggcctgagcaaagaggccctgatccaggccttgggggagccagggccctacctg
atggttggagagggtcccctggacaagggggagtcctgcggtgggagtcgaggggacctgaccgagtt
gcctaatggccttggagaaacgcgtggctctgaagatgacacggatgacgatggggaagacttcgcgc
cacccattctgaaagagctggagaacctcagcccagaggaggcagcccaccagaaagccgtggtggag
tcacttcttcaggaggacccatggcgcgtggcgaagatggtcaagtcgtacttgcagcagcacaacat
cccccagcgggaggtggtggacaccacgggtctcaaccagtcccacctgtcacagcacctcaacaagg
gcacacccatgaagacacagaagcgggccgctctgtacacctggtacgtccgcaagcagcgagaggtg
gctcagcaattcacccacgcagggcagggcggactgattgaagagcccacaggcgatgagctgccaac
taagaaggggcgtaggaaccggttcaagtggggccccgcatcccagcagatcctgttccaggcctacg
agaggcaaaaaaaccccagcaaggaagagcgagagaccttggtggaggagtgtaatagggcggagtgc
atccagaggggggtgtcaccatcgcaggcccaggggctaggctccaaccttgtcacggaggtgcgtgt
ctacaactggtttgccaaccggcgcaaggaggaagccttccggcacaagttggccatggacacctata
acggacctccaccggggccaggcccgggccctgcgctgcctgctcacagttcccccggcctgcccaca
accaccctctctcccagtaaggtccacggtgtacggtacggacagtctgcaaccagtgaggcagccga
ggtgccctccagcagcggaggtcccttagtcacagtgtctgcggccttacaccaagtatcccccacag
gcctggagcccagcagcctgctgagcacagaggccaagctggtctcagccacggggggtcccctgcct
cccgtcagcaccctgacagcactgcacagcttggagcagacatctccgggtctcaaccagcagccgca
gaaccttatcatggcctcgctacctggggtcatgaccatcgggcccggggagcctgcctccctgggac
ccacgttcacgaacacgggcgcctccaccctggttatcggtctggcctccactcaggcacagagcgtg
cctgtcatcaacagcatggggagtagcctgaccacgctgcagccggtccagttttcccaaccactgca
tccctcctatcagcagcctctcatgccccccgtacagagccacgtggcccagagccccttcatggcaa
ccatggcccagctgcagagcccccacgccttatacagccacaagcctgaggtggcccagtacacgcac
accagcctgctcccgcagaccatgttgatcacagacaccaacctcagcacccttgccagcctcacacc
caccaagcaggtcttcacctcagacacagaggcctccagtgagcccgggcttcacgagccaccctctc
cagccaccaccatccacatccccagccaggacccgtcgaacatccagcacctgcagcctgctcaccgg
ctcagcaccagtcccacagtgtcctccagcagcctggtgttgtatcagagttccgactccaacgggca
cagccacctgctgccatccaaccatagtgtcatcgagacttttatctccacccagatggcctcctctt
cccagtaaggtaccgcggccgcgttaacacgcgtgatatccacgtggagctcgctgatcagcctcgac
tgtgccttctagttgccagccatctgttgtttgcccctcccccgtgccttccttgaccctggaaggtg
ccactcccactgtcctttcctaataaaatgaggaaattgcatcgcattgtctgagtaggtgtcattct
attctggggggtggggggggcaggacagcaagggggaggattgggaagacaatagcaggcatgctggg
gatgcggtgggctctatgg
5′ ITR: 1-128 bp
hINS1.9 promoter: 137-2036 bp
Mus musculus HNF1A_a coding sequence: 2067-3953 bp
Bovine growth hormonpe polyA: 4013-4237 bp
3′ ITR: 4253-4380 bp
hins385-HNF1A gene construct (SEQ ID NO: 50)
tgtggggacaggggtctggggacagcagcgcaaagagccccgccctgcagcctccagctctcctggtc
taatgtggaaagtggcccaggtgagggctttgctctcctggagacatttgcccccagctgtgagcagg
gacaggtctggccaccgggcccctggttaagactctaatgacccgctggtcctgaggaagaggtgctg
acgaccaaggagatcttcccacagacccagcaccagggaaatggtccggaaattgcagcctcagcccc
cagccatctgccgacccccccaccccaggccctaatgggccaggcggcaggggttgagaggtagggga
gatgggctctgagactataaagccagcgggggcccagcagccctcgatatcctcgaggctagcgaatt
cgccaccatggtttctaagctgagccagctgcagacggagctcctggctgccctgctcgagtctggcc
tgagcaaagaggccctgatccaggccttgggggagccagggccctacctgatggttggagagggtccc
ctggacaagggggagtcctgcggtgggagtcgaggggacctgaccgagttgcctaatggccttggaga
aacgcgtggctctgaagatgacacggatgacgatggggaagacttcgcgccacccattctgaaagagc
tggagaacctcagcccagaggaggcagcccaccagaaagccgtggtggagtcacttcttcaggaggac
ccatggcgcgtggcgaagatggtcaagtcgtacttgcagcagcacaacatcccccagcgggaggtggt
ggacaccacgggtctcaaccagtcccacctgtcacagcacctcaacaagggcacacccatgaagacac
agaagcgggccgctctgtacacctggtacgtccgcaagcagcgagaggtggctcagcaattcacccac
gcagggcagggggactgattgaagagcccacaggcgatgagctgccaactaagaaggggcgtaggaac
cggttcaagtggggccccgcatcccagcagatcctgttccaggcctacgagaggcaaaaaaaccccag
caaggaagagcgagagaccttggtggaggagtgtaatagggcggagtgcatccagaggggggtgtcac
catcgcaggcccaggggctaggctccaaccttgtcacggaggtgcgtgtctacaactggtttgccaac
cggcgcaaggaggaagccttccggcacaagttggccatggacacctataacggacctccaccggggcc
aggcccgggccctgcgctgcctgctcacagttcccccggcctgcccacaaccaccctctctcccagta
aggtccacggtgtacggtacggacagtctgcaaccagtgaggcagccgaggtgccctccagcagcgga
ggtcccttagtcacagtgtctgcggccttacaccaagtatcccccacaggcctggagcccagcagcct
gctgagcacagaggccaagctggtctcagccacggggggtcccctgcctcccgtcagcaccctgacag
cactgcacagcttggagcagacatctccgggtctcaaccagcagccgcagaaccttatcatggcctcg
ctacctggggtcatgaccatcgggcccggggagcctgcctccctgggacccacgttcacgaacacggg
cgcctccaccctggttatcggtctggcctccactcaggcacagagcgtgcctgtcatcaacagcatgg
ggagtagcctgaccacgctgcagccggtccagttttcccaaccactgcatccctcctatcagcagcct
ctcatgccccccgtacagagccacgtggcccagagccccttcatggcaaccatggcccagctgcagag
cccccacgccttatacagccacaagcctgaggtggcccagtacacgcacaccagcctgctcccgcaga
ccatgttgatcacagacaccaacctcagcacccttgccagcctcacacccaccaagcaggtcttcacc
tcagacacagaggcctccagtgagcccgggcttcacgagccaccctctccagccaccaccatccacat
ccccagccaggacccgtcgaacatccagcacctgcagcctgctcaccggctcagcaccagtcccacag
tgtcctccagcagcctggtgttgtatcagagttccgactccaacgggcacagccacctgctgccatcc
aaccatagtgtcatcgagacttttatctccacccagatggcctcctcttcccagtaaggtaccgcggc
cgcgttaacacgcgtgatatccacgtggagctcgctgatcagcctcgactgtgccttctagttgccag
ccatctgttgtttgcccctcccccgtgccttccttgaccctggaaggtgccactcccactgtcctttc
ctaataaaatgaggaaattgcatcgcattgtctgagtaggtgtcattctattctggggggtggggggg
gcaggacagcaagggggaggattgggaagacaatagcaggcatgctggggatgcggtgggctctatgg
5′ ITR: 1-128 bp
hINS385 promoter: 137-521 bp
Mus musculus HNF1A_a coding sequence: 552-2438 bp
Bovine growth hormonpe polyA: 2498-2722 bp
3′ ITR: 2738-2865 bp

1. A gene construct for expression in the pancreas comprising a nucleotide sequence encoding a hepatocyte nuclear factor (HNF), operably linked to:
(a) a pancreas-specific promoter; or
(b) a ubiquitous promoter and at least one target sequence of a microRNA expressed in non-pancreatic tissue.

2. The gene construct according to claim 1, wherein the pancreas-specific promoter is selected from the group consisting of the pancreas/duodenum homeobox protein 1 (Pdx1) promoter, neurogenin 3 (Ngn3) promoter, HNF promoters, elastase I promoter, amylase promoter, MafA promoter, insulin (Ins) promoter and derivatives thereof, preferably wherein the pancreas-specific promoter is an insulin promoter or a derivative thereof.

3. The gene construct according to claim 2, wherein the pancreas-specific promoter is a murine, canine or human insulin promoter or a derivative thereof, preferably a human or murine insulin promoter or a derivative thereof, more preferably a human insulin promoter or a derivative thereof.

4. The gene construct according to claim 3, wherein the pancreas-specific promoter comprises, consists essentially of or consists of:
the nucleotides corresponding to positions −385 to −1 in the human insulin promoter (SEQ ID NO: 18); and/or
the nucleotide sequence of SEQ ID NO: 20, or a sequence having at least 60%, 70%, 80%, 90%, 95% or 99% sequence identity therewith.

5. The gene construct according to claim 1, wherein the at least one target sequence of a microRNA is selected from those target sequences that bind to microRNAs expressed in heart and/or liver.

6. The gene construct according to claim 5, wherein the gene construct comprises at least one target sequence of a microRNA expressed in the liver and at least one target sequence of a microRNA expressed in the heart, preferably wherein a target sequence of a microRNA expressed in the heart is selected from SEQ ID NO's: 29-34, and a target sequence of a microRNA expressed in the liver is selected from SEQ ID NO's: 21-28, more preferably wherein the gene construct comprises a target sequence of microRNA-122a (SEQ ID NO: 21) and a target sequence of microRNA-1 (SEQ ID NO: 29).

7. The gene construct according to claim 1, wherein the HNF is an HNF1A.

8. The gene construct according to claim 7, wherein the nucleotide sequence encoding HNF1A is selected from the group consisting of:
(a) a nucleotide sequence encoding a polypeptide represented by an amino acid sequence comprising a sequence that has at least 60%, 70%, 80%, 90%, 95% or 99% sequence identity or similarity with the amino acid sequence of any one of SEQ ID NO: 1-11,51;
(b) a nucleotide sequence that has at least 60%, 70%, 80%, 90%, 95% or 99% sequence identity with the nucleotide sequence of SEQ ID NO: 12-15; and
(c) a nucleotide sequence the sequence of which differs from the sequence of a nucleotide sequence of (b) due to the degeneracy of the genetic code.

9. An expression vector comprising a gene construct as described in claim 1.

10. The expression vector according to claim 9, wherein the expression vector is a viral vector, preferably an adeno-associated viral vector.

11. The expression vector according to claim 9, wherein the expression vector is an adeno-associated viral vector of serotype 1, 2, 3, 4, 5, 6, 7, 8, 9, rh10, rh8, Cb4, rh74, DJ, 2/5, 2/1, 1/2 or Anc80, preferably an adeno-associated viral vector of serotype 6, 8 or 9, more preferably an adeno-associated viral vector of serotype 8.

12.-15. (canceled)

16. A method for treating maturity onset diabetes of the young (MODY) or a condition associated therewith, the method comprising administering the expression vector according to claim 9.

17. The method according to claim 14, wherein MODY is MODY3 or a condition associated therewith.


SEQUENCE LISTING (65 sequences)

SEQ ID NO: 1 - protein, 631 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVFTSDTEASSESGLHTPASQATTLHVPSQDPAGIQHLQPAHRLSASPTVSSSSLVLYQSSDSSNGQSHLLPSNHSVIETFISTQMASSSQ

SEQ ID NO: 2 - protein, 572 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHGEHPVPHTAGDDDRGWLSMDAGERGAWQALQSACVSGTSVFPDSSNGQSHLLPSNHSVIETFISTQMASSSQ

SEQ ID NO: 3 - protein, 524 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGKLVGMGGHLGGRLMGQPQNPGAGRATGTHSFIHSFIQHVFIQCLLWTSHCATSVIPGDSSNGQSHLLPSNHSVIETFISTQMASSSQ

SEQ ID NO: 4 - protein, 278 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQRRNASREGCPHHRHRGWAPTSSRRCVSTTGLPTGAKKKPSGTSWPWTRTAGPPQGQARDLRCPLTAPLACLHLPSPPVRSTVCAMDSLRPVRLQKYPQAAAVP

SEQ ID NO: 5 - protein, 247 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRCALWTACDQ

SEQ ID NO: 6 - protein, 403 aa, Homo sapiens
MVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGKLVGMGGHLGGRLMGQPQNPGAGRATGTHSFIHTTCIYPVPTLDQSLCYISDTWVNQTDQNLSNSSREAGTKHNTSILWYLRR

SEQ ID NO: 7 - protein, 662 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYSGPPPGPGPGPALPAHSSPGLPPPALSPSKVHGVRYGQPATSETAEVPSSSGGPLVTVSTPLHQVSPTGLEPSHSLLSTEAKLVSAAGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVTQSPFMATMAQLQSPHALYSHKPEVAQYTHTGLLPQTMLITDTTNLSALASLTPTKQVRSRPAGPPLACDRAPHPHIPRAQEAALLPQVFTSDTEASSESGLHTPASQATTLHVPSQDPAGIQHLQPAHRLSASPTVSSSSLVLYQSSDSSNGQSHLLPSNHSVIETFISTQMASSSQ

SEQ ID NO: 8 - protein, 119 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQVHPCRAGRAD

SEQ ID NO: 9 - protein, 247 aa, Mus musculus
MVSKLSQLQTELLAALLESGLSKEALIQALGEPGPYLMVGEGPLDKGESCGGSRGDLTELPNGLGETRGSEDDTDDDGEDFAPPILKELENLSPEEAAHQKAVVESLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRCTVRTVCNQ

SEQ ID NO: 10 - protein, 119 aa, Mus musculus
MVSKLSQLQTELLAALLESGLSKEALIQALGEPGPYLMVGEGPLDKGESCGGSRGDLTELPNGLGETRGSEDDTDDDGEDFAPPILKELENLSPEEAAHQKAVVESLLQIHPRRAGRTD

SEQ ID NO: 11 - protein, 631 aa, Canis lupus familiaris
MVSKLSQLQTELLAALLHSGLSKEALIQALGEPGPYLLAGDGPLDKGESCGGGRGELAELPNGLGEARGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEEREALVEECNRAECIQRGVSPSQAQGLGSNLVTEVRVYNWFANRRKEEAFRHKLAMDTYSGPPPGPGPGPTLPAHSSPGLPPPALSPSKVHGVRYGQQATSEGAEVPSSSGGPLVTVSAPLHQVSPTGLEPSHSLLSTEAKLVSATGGPLPPVSTLTALHSLEQTSPGLNQQPQNLIMASLPGVMAIGPGEPASLGPTFTNTGASTLVIGLASTQAQSVPVINSMGSSLTTLQPVQFSQPLHPSYQQPLMSPMQSHVAQSPFMATMAQIQSPHALYSHKPEVAQYTHTGLLPQTMLITDTTNLSALASLTPTKQVFTSDAEASSESGLHTPASQTTTIHIPSQDPVGIQHLQPAHRLSASPTVSSSSLVLYQSSDSNNGHSHLLPSNHSVIETFISTQMASSSQ

SEQ ID NO: 12 - DNA, 1896 bp, Homo sapiens
atggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgagacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagacgctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatggacacgtacagcgggccccccccagggccaggcccgggacctgcgctgcccgctcacagctcccctggcctgcctccacctgccctctcccccagtaaggtccacggtgtgcgctatggacagcctgcgaccagtgagactgcagaagtaccctcaagcagcggcggtcccttagtgacagtgtctacacccctccaccaagtgtcccccacgggcctggagcccagccacagcctgctgagtacagaagccaagctggtctcagcagctgggggccccctcccccctgtcagcaccctgacagcactgcacagcttggagcagacatccccaggcctcaaccagcagccccagaacctcatcatggcctcacttcctggggtcatgaccatcgggcctggtgagcctgcctccctgggtcctacgttcaccaacacaggtgcctccaccctggtcatcggcctggcctccacgcaggcacagagtgtgccggtcatcaacagcatgggcagcagcctgaccaccctgcagcccgtccagttctcccagccgctgcacccctcctaccagcagccgctcatgccacctgtgcagagccatgtgacccagagccccttcatggccaccatggctcagctgcagagcccccacgccctctacagccacaagcccgaggtggcccagtacacccacacgggcctgctcccgcagactatgctcatcaccgacaccaccaacctgagcgccctggccagcctcacgcccaccaagcaggtcttcacctcagacactgaggcctccagtgagtccgggcttcacacgccggcatctcaggccaccaccctccacgtccccagccaggaccctgccagcatccagcacctgcagccggcccaccggctcagcgccagccccacagtgtcctccagcagcctggtgctgtaccagagctcagactccagcaatggccagagccacctgctgccatccaaccacagcgtcatcgagaccttcatctccacccagatggcctcttcctcccagtaa

SEQ ID NO: 13 - DNA, 1887 bp, Mus musculus
atggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaa

SEQ ID NO: 14 - DNA, 1896 bp, Canis lupus familiaris
atggtttctaaactgagccagctgcagacggagctcctggcggccctgctccactcgggcctgagcaaagaggcactgatccaggcgttgggtgagccggggccctacctgctggctggagatggccccctggacaagggggagtcctgcggcggcggtcggggggagctggccgagctgcccaacgggctgggggaggcgaggggctccgaggacgagacggacgacgatggggaagacttcacaccgcccatcctcaaggagctggagaacctcagccccgaggaggcggcccaccagaaagccgtggtggagaccttgctgcaggaggacccgtggcgcgtggccaagatggtcaaatcctacctgcagcagcataacatcccacagcgggaggtagtcgacaccactggcctcaaccagtcacacttgtcccagcatctcaacaagggcacccccatgaagacacagaagcgagctgccctgtacacctggtacgtccgcaagcagcgagaggtggcccagcagttcacccacgcagggcaaggtgggctgattgaagagcccacaggtgatgagctgccaaccaagaaggggcggaggaatcgtttcaagtggggcccagcctcccagcagatcctgttccaggcctatgagaggcagaagaaccccagcaaggaggagcgagaggcattggtggaggagtgcaatagggcggaatgcatccagaggggggtgtcaccatcgcaggcacaggggctgggctccaaccttgtcacagaggtgcgtgtctacaactggtttgccaatcgccgcaaggaagaagcctttcgtcataagctggccatggacacatacagcgggccaccacctgggccaggcccgggccccacactgcctgcccacagctctccgggcttgcccccacctgccctctcccctagtaaggtccacggtgtgcgctatggacagcaagcaactagcgagggggcggaagtgccttcgagcagcggtggtcccttggtgacggtgtctgcgcccctgcaccaagtgtcccccacaggcctggagcccagtcacagtctgctgagcaccgaagccaagctggtctcagcaactgggggccccttgccccctgtcagcaccctgacagcactgcacagcttggagcagacctccccaggcctcaaccaacagccccagaatctcatcatggcctcacttcccggggtcatggccattgggcctggtgagcctgcctccctgggccccacgttcaccaacacgggtgcctccaccctggtcattggcctggcctccactcaggcccagagcgtgccagtcatcaacagcatgggcagcagcctgaccaccctgcagccagtccagttctcccagccactgcacccttcctaccagcagccactcatgtctcccatgcagagccatgtggcccagagccccttcatggccaccatggcccagattcagagcccccatgccctctacagccacaagcccgaggtggcccagtacacccacacgggcctgcttccgcagaccatgctcatcaccgacactaccaacctgagcgccctggccagcctcacgcccaccaagcaggtcttcacctcagatgctgaggcctccagtgagtccgggcttcatacgccggcgtctcagaccaccaccatccacatccccagccaggatcctgttggcatccagcacctgcagcctgcccaccggctcagcgccagccccaccgtgtcctccagcagcctggtgttgtaccagagctccgactccaacaacggccacagccacctgttgccatccaaccacagcgtcatcgagaccttcatttccacgcagatggcttcatcctcccagtaa

SEQ ID NO: 15 - DNA, 1902 bp, Artificial sequence
  Codon optimized nucleotide sequence of homo sapiens HNF1A isoform a
gccaccatggtttccaagctgagccagctgcagaccgaactgctggctgctctgctggaaagcggcctgtctaaagaggccctgattcaggccctgggagagcctggaccttatctgcttgctggcgagggcccactggataagggcgaatcttgtggcggaggaagaggcgaactggccgagctgcctaatggactgggcgagacaagaggcagcgaggacgagacagatgacgacggcgaggatttcacccctcctatcctgaaagagctggaaaatctgagccccgaggaagccgctcaccagaaagctgtggtggaaaccctgctgcaagaggacccttggagagtggccaagatggtcaagagctacctgcagcagcacaacatccctcagcgcgaggtggtggataccacaggcctgaaccagagccacctgtctcagcacctgaacaagggcacccctatgaagacccagaagcgggctgccctgtacacttggtacgtgcggaagcagagagaggtggcccagcagtttacacacgctggacaaggcggactgatcgaggaacctacaggcgacgagctgcccaccaagaaaggcagacggaaccggtttaagtggggccctgcctctcagcagatcctgtttcaggcctacgagcggcagaagaaccccagcaaagaggaaagagagacactggtcgaggaatgcaaccgggccgagtgtatccagaggggcgttagtccttctcaggcccaaggcctgggcagcaatctggttacagaagtgcgggtctacaattggttcgccaaccggcggaaagaggaagccttcagacacaagctggccatggacacctacagcggacctccacctggaccaggacctggacctgcacttcctgctcattctagccctggactgcctcctcctgctctgtctccatctaaggtgcacggcgtcagatatggccagcctgccacatctgagacagccgaggttccatctagctctggcggacctctggtcaccgtgtctacacctctgcatcaggtgtccccaaccggcctggaaccttctcacagcctgctgagcacagaggccaaactggtgtcagctgcaggcggaccactgcctccagtctctacactgacagccctgcacagcctggaacagacaagccccggactgaatcagcagccccagaacctgattatggcctctctgcccggcgtgatgacaatcggacctggcgaacctgcttctctgggccccacctttacaaacaccggcgccagcacactggtcatcggactggcttctacacaggcccagagcgtgcccgtgatcaatagcatgggcagcagcctgaccacactgcagcctgtgcagtttagccagcctctgcacccctcttaccagcagcctctgatgccaccagtgcagagccacgtgacacagagccctttcatggccacaatggcccagctgcaaagccctcacgctctgtactctcacaagcccgaagtggcccagtacacccacacaggactgctgcctcagaccatgctgatcaccgacaccaccaatctgagcgccctggctagcctgacacctaccaaacaggtgttcaccagcgacaccgaggccagctctgaatctggactgcacacaccagccagccaggccacaacactgcatgtgccatctcaggaccctgccagcatccagcatctgcagccagctcacagactgagcgcctctccaacagtgtccagctctagcctggtgctgtaccagagcagcgacagcagcaatggccagagccatctgctgcctagcaaccacagcgtgatcgagacattcatcagcacccagatggccagcagcagccagtaa

SEQ ID NO: 16 - DNA, 539 bp, Artificial sequence
  rat insulin promoter 1
cgccgggttttgtggaagtagagatagaggagaagggaccattacatgtcctgctgcctgagttctgctttccttctccctttgaaggtgagctggggtctcagctgagctaagaatccagctatcaatagaaactatgaaacagttccagggacaaagataccaggtccccaacaactgcaactttctgggaaatgaggtggaaagtgctcagccaaggaaaaagagggccttaccctctctgggacaatgattgtgctgtgaactgcttcatcaggccatctggccccttgttaataatctaattaccctaggtctaagtagagttgctgacgtccaatgagcgctttctgcagacttagcactaggcaagtgtttggaaattacggcttcggcccctctcgccatctgcctacctacccctcctagagcccttaatgggccaaacggcaaagtccagggggcagagaggaggtgctttggactataaagctagtggagacccagtaactcccaaccctaacgggatatcctcgagt

SEQ ID NO: 17 - DNA, 721 bp, Artificial sequence
  rat insulin promoter 2
gatcccccaaccactccaagtggaggctgagaaaggttttgtagctgggtagagtatgtactaagagatggagacagctggctctgagctctgaagcaagcacctcttatggagagttgctgaccttcaggtgcaaatctaagatactacaggagaatacaccatggggcttcagcccagttgactcccgagtgggctatgggtttgtggaaggagagatagaagagaagggacctttcttcttgaattctgctttccttctacctctgagggtgagctggggtctcagctgaggtgaggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctattaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaagatatcctcgagtctag

SEQ ID NO: 18 - DNA, 1900 bp, Artificial sequence
  full-lenght human insulin promoter (hINS19)
ccccggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtgtggggacaggggtcctggggacaggggtctggggacaggggtcctggggacaggggtgtggggacaggggtgtggggacaggggtgtggggacaggggtcctggggacaggggtctggggacaggggtctgaggacaggggtgtggggacaggggtgtggggacaggggtgtggggacaggggtgtggggacaggggtctggggacaggggtccgggggacaggggtgtggggacaggggtgtggggacaggggtgtggggacaggggtctggggacaggggtgtggggacaggggtcctggggacaggggtgtggggataggggtgtggggacaggggtgtggggacaggggtgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctc

SEQ ID NO: 19 - DNA, 409 bp, Artificial sequence
  positions 385 to 24 in the human insulin promoter
tgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatca

SEQ ID NO: 20 - DNA, 385 bp, Artificial sequence
  positions 385 to 1 in the human insulin promoter
tgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctc

SEQ ID NO: 21 - DNA, 22 bp, Artificial
  Nucleotide sequence encoding miRT-122a
caaacaccattgtcacactcca

SEQ ID NO: 22 - DNA, 21 bp, Artificial sequence
  Nucleotide sequence encoding miRT-152
ccaagttctgtcatgcactga

SEQ ID NO: 23 - DNA, 23 bp, Artificial sequence
  Nucleotide sequence encoding miRT-199a-5p
gaacaggtagtctgaacactggg

SEQ ID NO: 24 - DNA, 22 bp, Artificial sequence
  Nucleotide sequence encoding miRT-199a-3p
taaccaatgtgcagactactgt

SEQ ID NO: 25 - DNA, 21 bp, Artificial sequence
  Nucleotide sequence encoding miRT-215
gtctgtcaattcataggtcat

SEQ ID NO: 26 - DNA, 21 bp, Artificial sequence
  Nucleotide sequence encoding miRT-192
ggctgtcaattcataggtcag

SEQ ID NO: 27 - DNA, 22 bp, Artificial sequence
  Nucleotide sequence encoding miRT-148a
acaaagttctgtagtgcactga

SEQ ID NO: 28 - DNA, 22 bp, Artificial sequence
  Nucleotide sequence encoding miRT-194
tccacatggagttgctgttaca

SEQ ID NO: 29 - DNA, 22 bp, Artificial sequence
  Nucleotide sequence encoding miRT-1
ttacatacttctttacattcca

SEQ ID NO: 30 - DNA, 22 bp, Artificial sequence
  Nucleotide sequence encoding miRT-133a
cagctggttgaaggggaccaaa

SEQ ID NO: 31 - DNA, 22 bp, Artificial
  Nucleotide sequence encoding miRT-206
ccacacacttccttacattcca

SEQ ID NO: 32 - DNA, 22 bp, Artificial sequence
  Nucleotide sequence encoding miRT-208a-5p
gtataacccgggccaaaagctc

SEQ ID NO: 33 - DNA, 22 bp, Artificial sequence
  Nucleotide sequence encoding miRT-208a-3p
acaagctttttgctcgtcttat

SEQ ID NO: 34 - DNA, 21 bp, Artificial
  Nucleotide sequence encoding miRT-499-5p
aaacatcactgcaagtcttaa

SEQ ID NO: 35 - DNA, 1671 bp, Artificial sequence
  Nucleotide sequence of CAG promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 36 - DNA, 212 bp, Artificial
  Nucleotide sequence of CMV promoter
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 37 - DNA, 133 bp, Artificial sequence
  Nucleotide sequence of chimeric intron composed of introns from human globin and immunoglobulin heavy chain genes
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 38 - DNA, 411 bp, Artificial sequence
  mini-CMV promoter
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagctt

SEQ ID NO: 39 - DNA, 1178 bp, Artificial sequence
  EF1alpha promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 40 - DNA, 623 bp, Artificial sequence
  RSV promoter
catgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagct

SEQ ID NO: 41 - DNA, 122 bp, Artificial sequence
  SV40 polyadenylation signal
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 42 - DNA, 448 bp, Artificial sequence
  Rabbit-globin polyadenylation signal
atctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 43 - DNA, 380 bp, Artificial sequence
  Nucleotide sequence of CMV enhancer
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 44 - DNA, 128 bp, Artificial sequence
  Truncated AAV2 5 ITR
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 45 - DNA, 128 bp, Artificial sequence
  Truncated AAV2 3 ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 46 - DNA, 592 bp, Artificial sequence
  CMV promoter and CMV enhancer sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 47 - DNA, 2730 bp, Artificial sequence
  RIPI-HNF1A gene construct
cgccgggttttgtggaagtagagatagaggagaagggaccattacatgtcctgctgcctgagttctgctttccttctccctttgaaggtgagctggggtctcagctgagctaagaatccagctatcaatagaaactatgaaacagttccagggacaaagataccaggtccccaacaactgcaactttctgggaaatgaggtggaaagtgctcagccaaggaaaaagagggccttaccctctctgggacaatgattgtgctgtgaactgcttcatcaggccatctggccccttgttaataatctaattaccctaggtctaagtagagttgctgacgtccaatgagcgctttctgcagacttagcactaggcaagtgtttggaaattacggcttcggcccctctcgccatctgcctacctacccctcctagagcccttaatgggccaaacggcaaagtccagggggcagagaggaggtgctttggactataaagctagtggagacccagtaactcccaaccctaacgggatgatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 48 - DNA, 2906 bp, Artificial sequence
  RIPII- HNF1A gene construct
ggatcccccaaccactccaagtggaggctgagaaaggttttgtagctgggtagagtatgtactaagagatggagacagctggctctgagctctgaagcaagcacctcttatggagagttgctgaccttcaggtgcaaatctaagatactacaggagaatacaccatggggcttcagcccagttgactcccgagtgggctatgggtttgtggaaggagagatagaagagaagggacctttcttcttgaattctgctttccttctacctctgagggtgagctggggtctcagctgaggtgaggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctattaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaagatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 49 - DNA, 4101 bp, Artificial sequence
  hIns1.9- HNF1A gene construct
ccccggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtgtggggacaggggtcctggggacaggggtctggggacaggggtcctggggacaggggtgtggggacaggggtgtggggacaggggtgtggggacaggggtcctggggacaggggtctggggacaggggtctgaggacaggggtgtggggacaggggtgtggggacaggggtgtggggacaggggtgtggggacaggggtctggggacaggggtccgggggacaggggtgtggggacaggggtgtggggacaggggtgtggggacaggggtctggggacaggggtgtggggacaggggtcctggggacaggggtgtggggataggggtgtggggacaggggtgtggggacaggggtgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcgatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 50 - DNA, 2586 bp, Artificial sequence
  hIns385- HNF1A gene construct
tgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcgatatcctcgaggctagcgaattcgccaccatggtttctaagctgagccagctgcagacggagctcctggctgccctgctcgagtctggcctgagcaaagaggccctgatccaggccttgggggagccagggccctacctgatggttggagagggtcccctggacaagggggagtcctgcggtgggagtcgaggggacctgaccgagttgcctaatggccttggagaaacgcgtggctctgaagatgacacggatgacgatggggaagacttcgcgccacccattctgaaagagctggagaacctcagcccagaggaggcagcccaccagaaagccgtggtggagtcacttcttcaggaggacccatggcgcgtggcgaagatggtcaagtcgtacttgcagcagcacaacatcccccagcgggaggtggtggacaccacgggtctcaaccagtcccacctgtcacagcacctcaacaagggcacacccatgaagacacagaagcgggccgctctgtacacctggtacgtccgcaagcagcgagaggtggctcagcaattcacccacgcagggcagggcggactgattgaagagcccacaggcgatgagctgccaactaagaaggggcgtaggaaccggttcaagtggggccccgcatcccagcagatcctgttccaggcctacgagaggcaaaaaaaccccagcaaggaagagcgagagaccttggtggaggagtgtaatagggcggagtgcatccagaggggggtgtcaccatcgcaggcccaggggctaggctccaaccttgtcacggaggtgcgtgtctacaactggtttgccaaccggcgcaaggaggaagccttccggcacaagttggccatggacacctataacggacctccaccggggccaggcccgggccctgcgctgcctgctcacagttcccccggcctgcccacaaccaccctctctcccagtaaggtccacggtgtacggtacggacagtctgcaaccagtgaggcagccgaggtgccctccagcagcggaggtcccttagtcacagtgtctgcggccttacaccaagtatcccccacaggcctggagcccagcagcctgctgagcacagaggccaagctggtctcagccacggggggtcccctgcctcccgtcagcaccctgacagcactgcacagcttggagcagacatctccgggtctcaaccagcagccgcagaaccttatcatggcctcgctacctggggtcatgaccatcgggcccggggagcctgcctccctgggacccacgttcacgaacacgggcgcctccaccctggttatcggtctggcctccactcaggcacagagcgtgcctgtcatcaacagcatggggagtagcctgaccacgctgcagccggtccagttttcccaaccactgcatccctcctatcagcagcctctcatgccccccgtacagagccacgtggcccagagccccttcatggcaaccatggcccagctgcagagcccccacgccttatacagccacaagcctgaggtggcccagtacacgcacaccagcctgctcccgcagaccatgttgatcacagacaccaacctcagcacccttgccagcctcacacccaccaagcaggtcttcacctcagacacagaggcctccagtgagcccgggcttcacgagccaccctctccagccaccaccatccacatccccagccaggacccgtcgaacatccagcacctgcagcctgctcaccggctcagcaccagtcccacagtgtcctccagcagcctggtgttgtatcagagttccgactccaacgggcacagccacctgctgccatccaaccatagtgtcatcgagacttttatctccacccagatggcctcctcttcccagtaaggtaccgcggccgcgttaacacgcgtgatatccacgtggagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 51 - protein, 628 aa, Mus musculus
MVSKLSQLQTELLAALLESGLSKEALIQALGEPGPYLMVGEGPLDKGESCGGSRGDLTELPNGLGETRGSEDDTDDDGEDFAPPILKELENLSPEEAAHQKAVVESLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMDTYNGPPPGPGPGPALPAHSSPGLPTTTLSPSKVHGVRYGQSATSEAAEVPSSSGGPLVTVSAALHQVSPTGLEPSSLLSTEAKLVSATGGPLPPVSTLTALHSLEQTSPGLNQQPQNLIMASLPGVMTIGPGEPASLGPTFTNTGASTLVIGLASTQAQSVPVINSMGSSLTTLQPVQFSQPLHPSYQQPLMPPVQSHVAQSPFMATMAQLQSPHALYSHKPEVAQYTHTSLLPQTMLITDTNLSTLASLTPTKQVFTSDTEASSEPGLHEPPSPATTIHIPSQDPSNIQHLQPAHRLSTSPTVSSSSLVLYQSSDSNGHSHLLPSNHSVIETFISTQMASSSQ

SEQ ID NO: 52 - DNA, 22 bp, Artificial sequence
  Target sequence of mir-375
tctcgcgagccgaacgaacaaa

SEQ ID NO: 53 - RNA, 20 nt, Artificial sequence
  sgRNA for MODY3 model
ccuccucuucccaguaaccg

SEQ ID NO: 54 - DNA, 20 bp, Artificial sequence
  Forward primer
ggacttggccaacagctagt

SEQ ID NO: 55 - DNA, 20 bp, Artificial sequence
  Reverse primer
ggaggagcagcagtgtcaat

SEQ ID NO: 56 - DNA, 20 bp, Artificial sequence
  Rplp0 forward primer
tcccaccttgtctccagtct

SEQ ID NO: 57 - DNA, 21 bp, Artificial sequence
  Rplp0 reverse primer
actggtctaggacccgagaag

SEQ ID NO: 58 - DNA, 20 bp, Artificial sequence
  L-PK forward primer
gtttcttgggcaacaggaag

SEQ ID NO: 59 - DNA, 20 bp, Artificial sequence
  L-PK reverse primer
aggaggcaaagatgatgtcc

SEQ ID NO: 60 - DNA, 20 bp, Artificial sequence
  HNF4a forward primer
agattgacaacctgctgcag

SEQ ID NO: 61 - DNA, 20 bp, Artificial sequence
  HNF4a reverse primer
tgcccatgtgttcttgcatc

SEQ ID NO: 62 - DNA, 20 bp, Artificial sequence
  HNF1a forward primer
tgtcacagcacctcaacaag

SEQ ID NO: 63 - DNA, 20 bp, Artificial sequence
  HNF1a reverse primer
tgtgggctcttcaatcagtc

SEQ ID NO: 64 - DNA, 20 bp, Artificial sequence
  Slc2a2 forward primer
atcccttggttcatggttgc

SEQ ID NO: 65 - DNA, 20 bp, Artificial sequence
  Slc2a2 reverse primer
aattgcagacccagttgctg